(19) United States
(12) Patent Application Publication
(10) Pub. No.: US 2024/0279723 A1
(43) Pub. Date: Aug. 22, 2024

(54) COMPOSITIONS AND METHODS FOR IN SITU SINGLE CELL ANALYSIS USING ENZYMATIC NUCLEIC ACID EXTENSION

(71) Applicant: NanoString Technologies, Inc., Seattle, WA (US)

(72) Inventors: Lidan WU, Seattle, WA (US); Rustem KHAFIZOV, Seattle, WA (US); Evan Paul PERILLO, Woodinville, WA (US); Dwayne DUNAWAY, Seattle, WA (US); Dae KIM, Bellevue, WA (US); Joseph M. BEECHEM, Piedmont, CA (US)

(21) Appl. No.: 18/570,190

(22) PCT Filed: Jun. 17, 2022
(86) PCT No.: PCT/US2022/033937

§ 371 Date: Dec. 14, 2023

Related U.S. Application Data
(60) Provisional application No. 63/211,666, filed on Jun. 17, 2021.

(51) Int. Cl. C12Q 1/6841; C12Q 1/48; C12Q 1/6806; C12Q 1/6874

(52) U.S. Cl. CPC C12Q 1/6841; C12Q 1/485; C12Q 1/6806; C12Q 1/6874; G01N 2333/9126

(57) ABSTRACT

The present disclosure is based in part on probes, compositions, methods, and kits for simultaneous, multiplexed spatial detection and quantification of protein and/or nucleic acid expression in a user-defined region of a tissue, user-defined cell, and/or user-defined subcellular structure within a cell.

### SEQUENCE LISTING

This application claims priority to, and the benefit of, U.S. Provisional Application No. 63/211,666, filed on Jun. 17, 2021. The contents of the aforementioned patent application are incorporated herein by reference in their entirety, for all purposes.

### SEQUENCE LISTING

The instant application contains a Sequence Listing which has been submitted in ASCII format via EFS-Web and is hereby incorporated by reference in its entirety. Said ASCII copy, created on Jun. 9, 2022, is named “NATE-052_001 WO_SeqList.txt” and is about 2,051,371 bytes in size.

### BACKGROUND OF THE INVENTION

Standard immunohistochemical and in situ hybridization methods allow for simultaneous detection of, at most, six to ten protein or nucleic acid targets, with three to four targets being typical. There exists a need for probes, compositions, methods, and kits for simultaneous, multiplexed detection and quantification of protein and/or nucleic acid expression in a user-defined region of a tissue, user-defined cell, and/or user-defined subcellular structure within a cell at a high enough resolution and with minimal background noise to do so. Furthermore, there is a need for such systems to be adaptable for use with existing sequencing technologies that are already available to a large number of end users.

### SUMMARY OF THE INVENTION

The present disclosure provides methods for in situ synthesis of a nucleic acid sequence in a tissue sample, the methods comprising: a) contacting the tissue sample with at least one probe, wherein the probe comprises a target-binding domain and a target identification domain, wherein the probe comprises a free 3′-OH moiety, and wherein the target-binding domain binds to at least one target molecule located at a first location of the tissue sample; b) contacting the tissue sample with at least one reversible terminator nucleotide, at least one polymerase, at least one caged chelator-cofactor complex, and at least one unbound caged chelator, wherein the at least one caged chelator-cofactor complex comprises at least one cofactor bound to a caged chelator; wherein the at least one reversible terminator nucleotide comprises the nucleotide operably linked to a cleavable 3′ terminator moiety; c) illuminating the first location of the tissue sample with light sufficient to uncage the at least one caged chelator-cofactor complex, thereby releasing the at least one cofactor, thereby activating the at least one polymerase, thereby ligating the at least one reversible terminator nucleotide to the free 3′-OH moiety of the at least one bound probe; d) washing the tissue sample; e) treating the tissue sample under conditions sufficient to cleave the 3′ terminator moiety of the at least one reversible terminator nucleotide ligated in step (c), thereby exposing a free 3′-OH moiety on the extended at least one bound probe; and f) repeating steps (b)-(e) until the nucleic acid sequence has been synthesized.

In some aspects of the preceding methods, a target-binding domain can bind to at least one target molecule at an at least second location of the tissue sample, and the methods can further comprise repeating steps (b)-(f) at the at least second location.

In some aspects of the preceding methods, a nucleic acid sequence synthesized at a first location of the tissue sample can be different than a nucleic acid sequence synthesized at an at least second location of the tissue sample.

The present disclosure provides methods of producing a spatially-resolved profile of the abundance of at least two target analytes in a first and an at least second location of a tissue sample comprising: a) contacting the tissue sample with a solution comprising at least two species of probes, the probes comprising a target-binding domain and a target identification domain, wherein each species of probe comprises a unique target-binding domain that binds to one of the at least two target analytes and a unique target identification domain specific for the target analyte, and a free 3′-OH moiety; b) contacting the tissue sample with a first plurality of reversible terminator nucleotides, a first plurality of polymerases, a first plurality of caged chelator-cofactor complexes, and a first plurality of unbound caged chelator, wherein at least one caged chelator-cofactor complex in the first plurality comprises at least one cofactor bound to a caged chelator; wherein at least one reversible terminator nucleotide in the first plurality comprises the nucleotide operably linked to a cleavable 3′ terminator moiety; c) illuminating at least one location of the tissue sample with light sufficient to uncage the caged chelator-cofactor complexes, thereby releasing the cofactors at the at least one location of the tissue sample, thereby activating the polymerases at the at least one location of the tissue sample, thereby ligating a reversible terminator nucleotide to the free 3′-OH moiety of at least one probe bound to a target analyte at the at least one location of the tissue sample, thereby extending the at least one bound probe; d) washing the tissue sample; e) treating the tissue sample under conditions sufficient to cleave the 3′ terminator moiety of the at least one reversible terminator nucleotide ligated in step (c), thereby exposing a free 3′-OH moiety on the extended at least one bound probe; f) repeating steps (b)-(e) until at least one probe bound to a target analyte in the first location of the tissue sample has been extended such that a first spatial barcode domain has been synthesized, wherein the first spatial barcode domain comprises a unique nucleic acid sequence specific to the first location of the tissue sample, at least one probe bound to a target analyte in the at least second location of the tissue sample has been extended such that a second spatial barcode domain has been synthesized, wherein the second spatial barcode domain comprises a unique nucleic acid sequence specific to the at least second location of the tissue sample; g) collecting the probes bound to target analytes in the tissue sample; and h) quantifying via sequencing the probes collected in step (f), thereby determining the abundance of the at least two target analytes in the first and the at least second location of the tissue sample, thereby producing a spatially-resolved profile of the abundance of the at least two target analytes.

In some aspects, the preceding methods can further comprise comparing the abundance of at least two target analytes in a first location of the tissue sample and at least two target analytes in at least a second location of the tissue sample.

In some aspects of the preceding methods, a polymerase can be a terminal deoxynucleotidyl transferase or a biologically active fragment thereof.

In some aspects of the preceding methods, a 3′ terminator moiety can be a 3′-ONH₂ group.

In some aspects of the preceding methods, a caged-chelator can be a caged divalent cation chelator. In some aspects, a caged-chelator can be 1-(4,5-Dimethoxy-2-Nitrophenyl)-1,2-Diaminoethane-N,N,N′,N′-Tetraacetic Acid (DMNP-EDTA).

In some aspects of the preceding methods, a cofactor can be a divalent metal cofactor. In some aspects of the preceding methods, a cofactor can be Co²⁺, Mg²⁺, Mn²⁺ Ca²⁺, Cd²⁺, Zn²⁺ or Fe²⁺. In some aspects of the preceding methods, a cofactor can be Co²⁺.

In some aspects of the preceding methods, contacting a tissue sample with a cofactor can comprise contacting the tissue sample with a salt form of the cofactor.

In some aspects of the preceding methods, light sufficient to uncage the caged chelator-cofactor complex can be UV light.

In some aspects of the preceding methods, treating a tissue sample under conditions sufficient to cleave a 3′ terminator moiety of an at least one reversible terminator nucleotide can comprise treating the tissue sample under acidic conditions. In some aspects of the preceding methods, treating a tissue sample under acidic conditions comprises contacting the tissue sample with a solution with a pH of about 5.5.

In some aspects of the preceding methods, a probe can further comprise a unique molecular identifier.

In some aspects of the preceding methods, a probe can further comprise an amplification primer binding site. In some aspects, an amplification primer binding site can be at least about 24 nucleotides in length.

In some aspects of the preceding methods, a probe can further comprise a constant region. In some aspects, a constant region can be at least about 12 to at least about 20 nucleotides in length.

In some aspects of the preceding methods, a probe can comprise, from 5′ to 3′, a target binding domain, followed by an amplification primer binding site, followed by a unique molecular identifier, followed by a target identification domain, followed by a constant region.

In some aspects of the preceding methods, a spatial barcode domain of at least one probe bound to a target analyte in a first location of the tissue sample comprises a unique spatial identifier sequence specific to the first location of the tissue sample. In some aspects of the preceding methods, a spatial barcode domain of at least one probe bound to a target analyte in an at least second location of the tissue sample comprises a unique spatial identifier sequence specific to the at least second location of the tissue sample.

In some aspects of the preceding methods, a spatial identifier sequence can comprise at least about 20 nucleotides. In some aspects of the preceding methods, a spatial identifier sequence can comprise at least about 28 nucleotides. In some aspects of the preceding methods, a spatial identifier sequence can comprise at least four spatial identification domains. In some aspects, the at least four spatial identification domains can comprise the same number of nucleotides. In some aspects, at least one of the at least four spatial identifications domains can comprise a different number of nucleotides as compared to another spatial identification domain within the same spatial barcode. In some aspects, each spatial identification domain can comprise about 1 to about 4 nucleotides. In some aspects, each spatial identification domain can comprise about 4 nucleotides. In some aspects, each of the at least four spatial identification domains can comprise the same nucleotide at the 3′ terminus.

In some aspects the preceding methods can further comprise, after step (f) and prior to step (g), repeating steps (b)-(e) to extend the spatial barcode domain in each location of the tissue sample such that the spatial barcode domain comprises, at the 3′ end, a delimiting domain.

In some aspects the preceding methods can further comprise, after step (f) and prior to step (g), extending the spatial barcode domain in each location of the tissue sample such that the spatial barcode domain comprises a polyT domain.

In some aspects the preceding methods can further comprise, after step (f) and prior to step (g): (i) repeating steps (b)-(e) to extend the spatial barcode domain in each location of the tissue sample such that the spatial barcode domain comprises, a delimiting domain; and (ii) extending the spatial barcode domain in each location of the tissue sample such that the spatial barcode domain comprises a polyT domain.

In some aspects of the preceding methods, a delimiting domain can be at least about 4 to at least about 6 nucleotides in length. In some aspects of the preceding methods, a sequence of a delimiting domain is the same for every spatial barcode in the sample.

In some aspects of the preceding methods, a polyT domain can comprise at least about 14 nucleotides.

In some aspects of the preceding methods, illumination can be provided by a light source selected from the group consisting of an arc-lamp, a laser, a focused UV light source, and light emitting diode. In some aspects of the preceding methods, the illumination can be UV illumination. In some aspects of the preceding methods, the illumination can be multiphoton illumination of a longer wavelength (e.g. two photon 780 nm laser).

In some aspects of the preceding methods, a first location of the tissue sample and an at least second location of the tissue sample can be subcellular. In some aspects, a first location of the tissue sample and an at least second location of the tissue sample can each comprise no more than one cell. In some aspects, a first location of the tissue sample and an at least second location of the tissue sample can each comprise no more than ten cells.

In some aspects the preceding methods, each cell within a first location of the tissue sample and an at least second location of the tissue sample can be individually automatically identified and encoded.

In some aspects the preceding methods can further comprise, prior to step (a), subjecting the tissue sample to ddTTP (dideoxthymidine-triphosphate) termination. In some aspects, subjecting a tissue sample to ddTTP termination can comprise contacting the tissue sample with ddTTP and TdT.

In some aspects the preceding methods can further comprise, after step (g) and prior to step (h), amplifying the collected probes. In some aspects, amplifying the collected probes can comprise the use of a first amplification primer and a second amplification primer, wherein the first amplification primer comprises a first flow cell adapter sequence, a first NGS index sequence and a first sequencing primer binding site, and the second amplification primer comprises a second flow cell adapter sequence, a second NGS index sequence and second sequencing primer binding site.

In some aspects of the preceding methods, at least one of the first and the second amplification primers comprises a nucleic acid sequence that is complementary to the delimiting sequence and/or the polyT domain.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 is an exemplary schematic of an in situ nucleic acid synthesis method of the present disclosure. In FIG. 1, a probe with a free 3′-OH, a TdT polymerase, a caged chelator-cofactor complex, reversible terminator nucleotides and unbound caged chelator are provided to a sample. Given that all of the cofactor is bound in the caged chelator-cofactor complex, there is no TdT activity, as the cofactor is required for TdT activity. In the next step, a first area is irradiated with UV light (represented by “hv” and the lightning bolt in FIG. 1), thereby releasing cofactor from the caged-chelator in the first area. The released cofactor can then bind to TdT, activating the polymerase, resulting in the addition of a reversible terminator nucleotide at the free 3′-OH of a probe located in the first location. In areas that were not exposed to UV light, the cofactor remains bound up in the caged chelator-cofactor complex, meaning that there is little to no TdT activation in those areas. Moreover, unbound caged chelator in the areas that were not exposed to UV light will chelate any cofactors that diffuse from locations that were irradiated with UV light. The entire sample is then washed to remove the reagents provided in the first step. The sample is then treated under mildly acidic conditions to cleave the 3′ terminator moiety of any reversible terminator nucleotides that were ligated onto ISH probes, thereby generating free 3′-OH groups that can be ligated to in subsequent cycles.

FIG. 2A-2Q is an exemplary schematic of the steps of the methods of producing a spatially-resolved profile of the abundance of at least one target analytes in at least two location of a tissue sample described in the present disclosure.

FIG. 2A shows an exemplary sample that can be analyzed using the methods of the present disclosure. In this non-limiting example shown in FIG. 2A-2Q, the sample comprises four cells, each cell comprising at least one copy of the specific target mRNA to be measured. Cell #1, Cell #2 and Cell #3 comprise one copy of the target mRNA and Cell #3 comprises two copies of the target mRNA.

FIG. 2B shows that the sample provided for use in the methods of the present disclosure can be subdivided into regions of interest (ROI) as shown in. In this non-limiting example, each ROI comprises one of the cells. Each ROI is pre-assigned a specific spatial barcode that is unique to that ROI. In this non-limiting example, each spatial barcode comprises 4 nucleotides, as shown in FIG. 2B.

FIG. 2C shows the first step of the methods of the present disclosure, wherein the sample is contacted with a plurality of probes, wherein the probes comprise a unique target-binding domain that binds to the target mRNA, a unique target identification domain specific for the target analyte, and a free 3′-OH moiety, as shown in the top left-hand corner of FIG. 2C.

FIG. 2D is a schematic showing the symbols used for various different reagents/molecules in FIGS. 2E-2Q, including reversible terminator nucleotides (comprising a cleavable 3′ terminator moiety), TdT polymerase, UV illumination, unbound caged chelator, caged chelator-cofactor complex, uncaged chelator, and released cofactor.

FIG. 2E shows the second step in the methods of the present disclosure, wherein the entire tissue sample is contacted with reversible terminator nucleotides, TdT polymerases, caged chelator-cofactor complexes, and unbound caged chelators. In this non-limiting example, the first reversible terminator nucleotides to be added are Adenine nucleotides (“A”).

FIG. 2F shows the third step in the methods of the present disclosure, wherein the ROI's that require the addition of an “A” nucleotide to construct the appropriate spatial barcode are illuminated with light of sufficient wavelength to uncage the caged chelator-cofactor complexes, thereby releasing cofactors. In this non-limiting example, ROI #1 require the addition of an “A” nucleotide, so ROI #1 is illuminated in this step. The illumination is represented by the lightning bolt shape in FIG. 2F. The result of this UV illumination is that, within the illuminated ROI #1, the caged chelator-cofactor complexes are uncaged, thereby releasing cofactor in ROI #1,

FIG. 2G shows that following release of the cofactor in ROI #1 (see FIG. 2F), the released cofactor binds to TdT molecules located in ROI #1, thereby activating the TdT molecules located in ROI #1.

FIG. 2H shows that the activated TdT molecules in ROI #1 will then proceed to ligate a reversible terminator nucleotide to the free 3′-OH groups of probes bound in ROI #1.

FIG. 2I shows the fourth step in the methods of the present disclosure, wherein the tissue sample is washed to remove unligated reversible terminator nucleotides, polymerases, caged chelator-cofactor complexes, unbound caged chelators, uncaged chelators, released cofactors.

FIG. 2J shows the fifth step in the methods of the present disclosure, wherein the tissue sample treated under conditions sufficient to cleave the cleavable 3′ terminator moiety of the ligated reversible terminator nucleotide, thereby exposing a free 3′-OH group on the probes bound to targets in ROI #1 for subsequent rounds of ligation.

FIG. 2K shows the next step in the methods of the present disclosure, wherein the entire tissue sample is contacted with reversible terminator nucleotides, TdT polymerases, caged chelator-cofactor complexes, and unbound caged chelators.

FIG. 2L shows the next step in the methods of the present disclosure, wherein the ROI's that require the addition of a “G” nucleotide to construct the appropriate spatial barcode are illuminated with light of sufficient wavelength to uncage the caged chelator-cofactor complexes, thereby releasing cofactors. In this non-limiting example, ROI #1 and ROI #4 require the addition of a “G” nucleotide, so ROI #1 and ROI #4 are illuminated in this step. The illumination is represented by the lightning bolt shape in FIG. 2L. The result of this UV illumination is that, within the illuminated ROI #1 and ROI #4, the caged chelator-cofactor complexes are uncaged, thereby releasing cofactor in ROI #1 and ROI #4.

FIG. 2M shows that following the release of the cofactor in ROI #1 and ROI #4 (see FIG. 2L), the released cofactor will then bind to TdT molecules located in ROI #1 and ROI #4, thereby activating the TdT molecules located in ROI #1 and ROI #4.

FIG. 2N shows that the activated TdT molecules in ROI #1 and ROI #4 will then proceed to ligate a reversible terminator nucleotide to the free 3′-OH groups of probes bound in ROI #1 and ROI #4.

FIG. 2O shows the next step in the methods of the present disclosure, wherein the tissue sample is washed to remove unligated reversible terminator nucleotides, polymerases, caged chelator-cofactor complexes, unbound caged chelators, uncaged chelators, released cofactors.

FIG. 2P shows the next step in the methods of the present disclosure, wherein the tissue sample is treated under conditions sufficient to cleave the cleavable 3′ terminator moiety of the ligated reversible terminator nucleotide, thereby exposing a free 3′-OH group on the probes bound to targets in ROI #1 and ROI #4 for subsequent rounds of ligation.

FIG. 2Q shows that the steps described in FIGS. 2A-2P can be repeated until all of the bound probes in each ROI have complete spatial barcodes.

FIG. 3A is an exemplary optical schematic of a two-photon excitation method that can be used in the methods of the present disclosure. A femtosecond pulsed (<100 fs) erbium laser emitting at 780 nm is scanned across the sample plane in a raster pattern with a pair of 2D galvanometric mirrors (1st and 2nd Scan Mirror). Digital image projection for photocleaving is generated by modulating the laser intensity at each pixel using a rapid optical shutter (acousto-optic modulator, AOM) with <100 ns rise/fall time. The output of the scanned laser is sent into a commercial EPI fluorescent microscope base and projected onto the sample with a 20× 1.0 NA water immersion objective.

FIG. 3B is an exemplary optical schematic of a two-photon excitation method that can be used in the methods of the present disclosure. A femtosecond pulsed (<100 fs) erbium laser emitting at 780 nm is scanned across the sample plane in a raster pattern with a pair of 2D galvanometric mirrors (SM1 and SM2). Digital image projection for photocleaving is generated by modulating the laser intensity at each pixel using a rapid optical shutter (acousto-optic modulator, AOM) with <100 ns rise/fall time. Polarization optics (HWP & GLP) and a negative group dispersion delay mirror pair (CMP) are used to cleanup polarization and compress the pulse at sample plane, respectively. The output of the scanned laser is sent into a commercial EPI fluorescent microscope base (Nikon Ti2) and projected onto the sample with a 40× 1.25 NA water immersion objective. The maximum field of view is 370×370 μm2, while the diffraction limited resolution is 380 nm. Achievable minimum cleaving feature resolution, measured as transition width from 10% to 90%, is less than 700 nm.

FIG. 4 is an exemplary schematic of a probe of the present disclosure, wherein the probe comprises a target binding domain, followed by an amplification primer binding site, followed by a Unique Molecular Identifier, followed by a Target Identification Domain.

FIG. 5 is an exemplary schematic of the probe of FIG. 4 that has been extended using TdT extension in the methods of the present disclosure to form a spatial barcode domain, wherein the spatial barcode domain comprises four spatial identification (ID) domains.

FIG. 6 is an exemplary schematic of the probe of FIG. 4 that has been extended using TdT extension in the methods of the present disclosure to form a spatial barcode domain, wherein the spatial barcode domain comprises four spatial identification (ID) domains and a delimiting domain.

FIG. 7 is exemplary schematic of the probe of FIG. 4 that has been extended using TdT extension in the methods of the present disclosure to form a spatial barcode domain, wherein the spatial barcode domain comprises four spatial identification (ID) domains, a delimiting domain and a polyT domain.

FIG. 8A is an exemplary schematic of an extended probe of the present disclosure, and two amplification primers of the present disclosure. The first amplification primer comprises a Flow Cell Adapter Sequence (e.g. P5), followed by an NGS Index Sequence (e.g. i5), followed by a sequencing primer binding site, followed by an amplification primer binding site that is complementary to and capable of hybridizing to the amplification primer binding site located on the probe with the spatial identifier sequence. The second amplification primer binding site comprises a polyA domain (which is capable of hybridizing to the polyT domain located on the probe), followed by a sequencing primer binding site, followed by an NGS index sequence (e.g. i7) followed by a Flow Cell Adapter Sequence (e.g. P7).

FIG. 8B is an exemplary schematic of an extended probe of the present disclosure and two amplification primers of the present disclosure. The first amplification primer comprises a Flow Cell Adapter Sequence (e.g. P5), followed by an NGS Index Sequence (e.g. i5), followed by a sequencing primer binding site, followed by an amplification primer binding site that is complementary to and capable of hybridizing to the amplification primer binding site located on the probe with the spatial identifier sequence. The second amplification primer binding site comprises two degenerate bases (“BN”) at the 3′ end, followed by a polyA domain (which is capable of hybridizing to the polyT domain located on the probe), followed by a sequencing primer binding site, followed by an NGS index sequence (e.g. i7) followed by a Flow Cell Adapter Sequence (e.g. P7).

FIG. 9 is an exemplary schematic of a probe of the present disclosure, wherein the probe comprises a target binding domain, followed by an amplification primer binding site, followed by a Unique Molecular Identifier, followed by a Target Identification Domain, followed by a constant region.

FIG. 10 is exemplary schematic of the probe of FIG. 9 that has been extended using TdT extension in the methods of the present disclosure to form a spatial barcode domain, wherein the spatial barcode domain comprises four identification (ID) domains, a delimiting domain and a polyT domain.

FIG. 11 is an exemplary schematic of a method of purifying the probes collected from a tissue sample as part of quantifying the probes via sequencing. The method comprises the use of a capture probe, wherein the capture probe comprises two copies of a sequence complementary to the amplification primer binding site present on the probe and a biotin molecule.

FIG. 12 is a series of images and graphs showing the analysis of in situ TdT-mediated nucleic acid extension performed using the methods of the present disclosure.

FIG. 13 is a graph showing the analysis of in situ TdT-mediated nucleic acid extension performed using the methods of the present disclosure. The extension was performed on a cell pellet array, as described in further detail in Example 1 below.

FIG. 14A and FIG. 14B show a series of tables depicting different experimental conditions under which in situ TdT-mediated nucleic acid extension was performed using the methods of the present disclosure.

FIG. 15 is a graph showing the analysis of in situ TdT-mediated nucleic acid extension performed using the methods of the present disclosure, as described in further detail in Example 1 below.

FIG. 16 is a graph showing the analysis of in situ TdT-mediated nucleic acid extension performed using the methods of the present disclosure, as described in further detail in Example 1 below.

### DETAILED DESCRIPTION OF THE INVENTION

The present disclosure is based in part on probes, compositions, methods, and kits for simultaneous, multiplexed spatial detection and quantification of protein and/or nucleic acid expression in a user-defined region of a tissue, user-defined cell, and/or user-defined subcellular structure within a cell using existing sequencing methods.

The present disclosure provides a comparison of the identity and abundance of target proteins and/or target nucleic acids present in a first region of interest (e.g., tissue type, a cell (including normal and abnormal cells), and a subcellular structure within a cell) and the identity and abundance of target proteins and/or target nucleic acids present in a second region of interest. There is no pre-defined upper limit to the number of regions of interest and comparisons that can be made; the upper limit relates to the size of the region of interest relative the size of the sample. As examples, when a single cell represents a region of interest, then a section may have hundreds to thousands of regions of interest; however, if a tissue section includes only two cell types, then the section may have only two regions of interest (each including only one cell type).

The present disclosure provides a higher degree of multiplexing than is possible with standard immunohistochemical or in situ hybridization methods. Standard immunohistochemical methods allow for maximal simultaneous detection of six to ten protein targets, with three to four protein targets being more typical. Similarly, in situ hybridization methods are limited to simultaneous detection of fewer than ten nucleic acid targets. The present disclosure provides detection of large combinations of nucleic acid targets and/or protein targets from a defined region of a sample. The present disclosure provides an increase in objective measurements by digital quantification and increased reliability and consistency, thereby enabling comparison of results among multiple centers.

As opposed to other methods known in the art, the methods of the present disclosure do not require sequential fluidic steps to extract probes/barcodes from specific regions of a tissue sample.

Moreover, as opposed to other methods known in the art that rely on digital micromirror-directed illumination systems, the methods of the present disclosure are compatible with two-photon illumination systems, including point scanning two-photon systems. The two-photon illumination systems exhibit increased spatial resolution, allowing the illumination of a single region of interest (ROI) that is as small as a single cell or even a subcellular structure within a single cell.

Moreover, the combination of TdT extension activation by any UV illumination system in the methods of the present disclosure can create a nonlinear effect (similar to two-photon, but chemically induced), allowing for increased spatial resolution, such that the illumination of a single region of interest (ROI) can be as small as a single cell or even a subcellular structure within a single cell.

Various compositions and methods of the present disclosure are described in full detail herein.

### Methods of the Present Disclosure

In some aspects, the methods described herein can be referred to as a “DNA writing microscope,” that can use a combination of two-photon microscopy and photo-activatable chemistry to create single-cell spatial barcodes in situ, thereby allowing for the quantification of gene-expression at single-cell resolution in a spatially resolved manner.

The present disclosure provides methods for in situ synthesis of a nucleic acid sequence in a tissue sample, the methods comprising: a) contacting the tissue sample with at least one probe, wherein the probe comprises a target-binding domain and a target identification domain, wherein the probe comprises a free 3′-OH moiety, and wherein the target-binding domain binds to at least one target molecule located at a first location of the tissue sample; b) contacting the tissue sample with at least one reversible terminator nucleotide, at least one polymerase, at least one caged chelator-cofactor complex, and at least one unbound caged chelator, wherein the at least one caged chelator-cofactor complex comprises at least one cofactor bound to a caged chelator; wherein the at least one reversible terminator nucleotide comprises the nucleotide operably linked to a cleavable 3′ terminator moiety; c) illuminating the first location of the tissue sample with light sufficient to uncage the at least one caged chelator-cofactor complex, thereby releasing the at least one cofactor, thereby activating the at least one polymerase, thereby ligating the at least one reversible terminator nucleotide to the free 3′-OH moiety of the at least one bound probe; d) washing the tissue sample; e) treating the tissue sample under conditions sufficient to cleave the 3′ terminator moiety of the at least one reversible terminator nucleotide ligated in step (c), thereby exposing a free 3′-OH moiety on the extended at least one bound probe; and f) repeating steps (b)-(e) until the nucleic acid sequence has been synthesized.

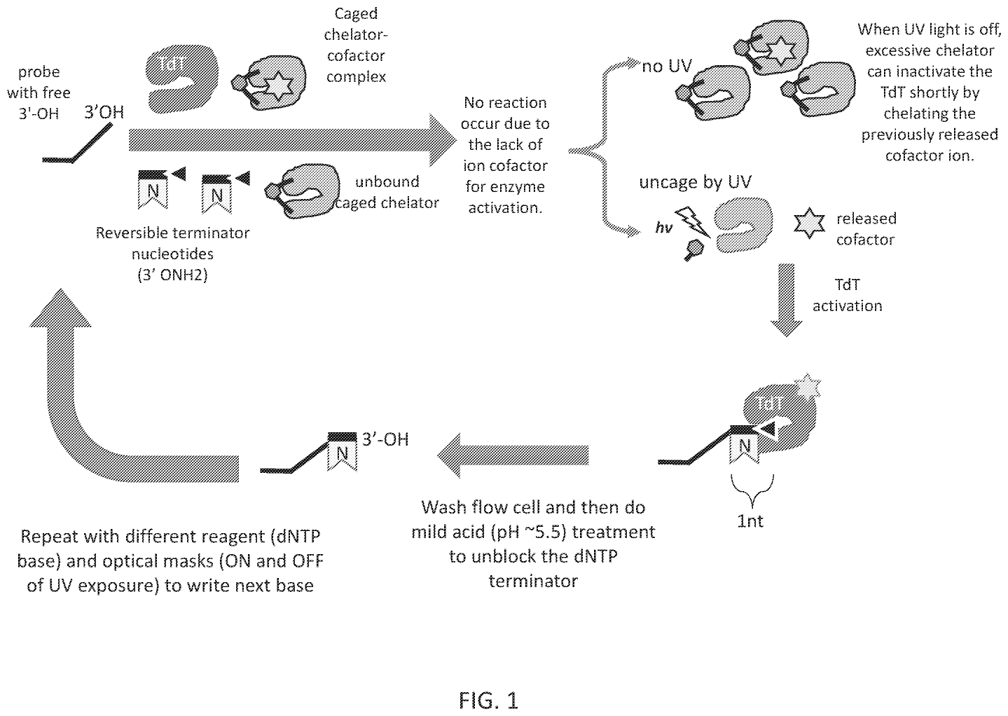

An exemplary schematic of the preceding method is shown in FIG. 1. In FIG. 1, a probe with a free 3′-OH, a TdT polymerase, a caged chelator-cofactor complex, reversible terminator nucleotides and unbound caged chelator are provided to a sample. Given that all of the cofactor is bound in the caged chelator-cofactor complex, there is no TdT activity, as the cofactor is required for TdT activity. In the next step, a first area is irradiated with UV light (represented by “hv” and the lightning bolt in FIG. 1), thereby releasing cofactor from the caged-chelator in the first area. The released cofactor can then bind to TdT, activating the polymerase, resulting in the addition of a reversible terminator nucleotide at the free 3′-OH of a probe located in the first location. In areas that were not exposed to UV light, the cofactor remains bound up in the caged chelator-cofactor complex, meaning that there is little to no TdT activation in those areas. Moreover, unbound caged chelator in the areas that were not exposed to UV light will chelate any cofactors that diffuse from locations that were irradiated with UV light. The entire sample is then washed to remove the reagents provided in the first step. The sample is then treated under mildly acidic conditions to cleave the 3′ terminator moiety of any reversible terminator nucleotides that were ligated onto ISH probes, thereby generating free 3′-OH groups that can be ligated to in subsequent cycles.

The present disclosure provides methods of producing a spatially-resolved profile of the abundance of at least two target analytes in a first and an at least second location of a tissue sample comprising: a) contacting the tissue sample with a solution comprising at least two species of probes, the probes comprising a target-binding domain and a target identification domain, wherein each species of probe comprises a unique target-binding domain that binds to one of the at least two target analytes and a unique target identification domain specific for the target analyte, and a free 3′-OH moiety; b) contacting the tissue sample with a first plurality of reversible terminator nucleotides, a first plurality of polymerases, a first plurality of caged chelator-cofactor complexes, and a first plurality of unbound caged chelator, wherein at least one caged chelator-cofactor complex in the first plurality comprises at least one cofactor bound to a caged chelator; wherein at least one reversible terminator nucleotide in the first plurality comprises the nucleotide operably linked to a cleavable 3′ terminator moiety; c) illuminating at least one location of the tissue sample with light sufficient to uncage the caged chelator-cofactor complexes, thereby releasing the cofactors at the at least one location of the tissue sample, thereby activating the polymerases at the at least one location of the tissue sample, thereby ligating a reversible terminator nucleotide to the free 3′-OH moiety of at least one probe bound to a target analyte at the at least one location of the tissue sample, thereby extending the at least one bound probe; d) washing the tissue sample; e) treating the tissue sample under conditions sufficient to cleave the 3′ terminator moiety of the at least one reversible terminator nucleotide ligated in step (c), thereby exposing a free 3′-OH moiety on the extended at least one bound probe; f) repeating steps (b)-(e) until at least one probe bound to a target analyte in the first location of the tissue sample has been extended such that a first spatial barcode domain has been synthesized, wherein the first spatial barcode domain comprises a unique nucleic acid sequence specific to the first location of the tissue sample, at least one probe bound to a target analyte in the at least second location of the tissue sample has been extended such that a second spatial barcode domain has been synthesized, wherein the second spatial barcode domain comprises a unique nucleic acid sequence specific to the at least second location of the tissue sample; g) collecting the probes bound to target analytes in the tissue sample; and h) quantifying via sequencing the probes collected in step (f), thereby determining the abundance of the at least two target analytes in the first and the at least second location of the tissue sample, thereby producing a spatially-resolved profile of the abundance of the at least two target analytes.

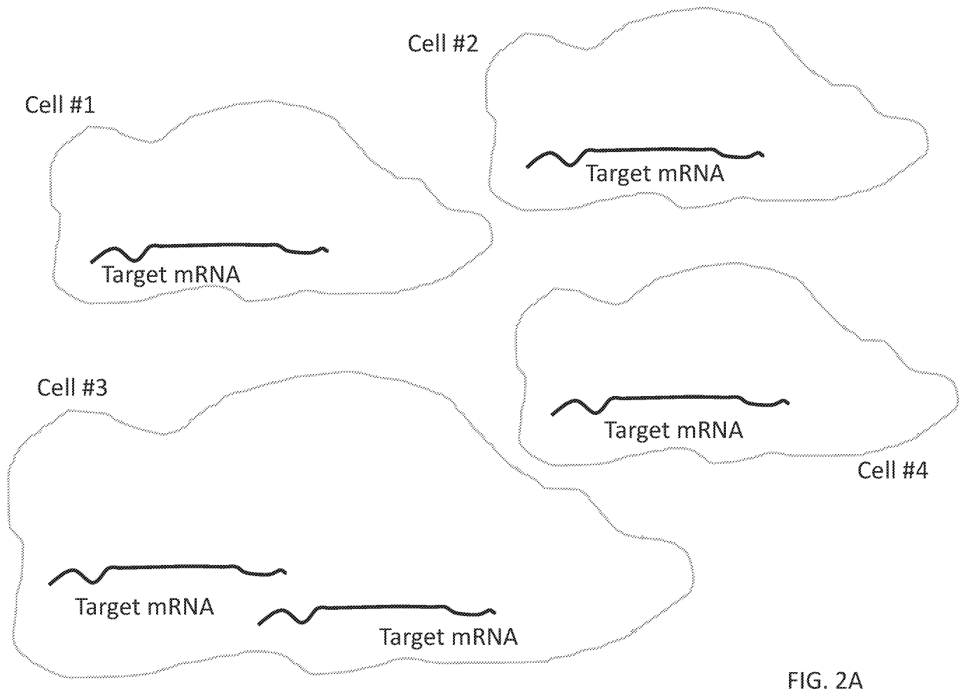

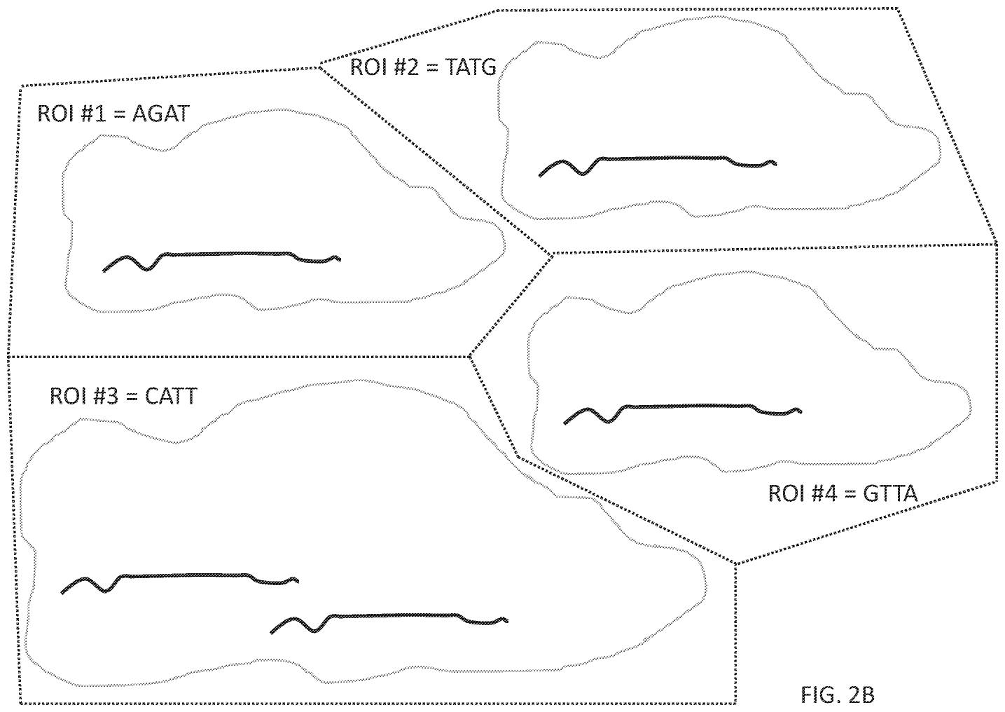

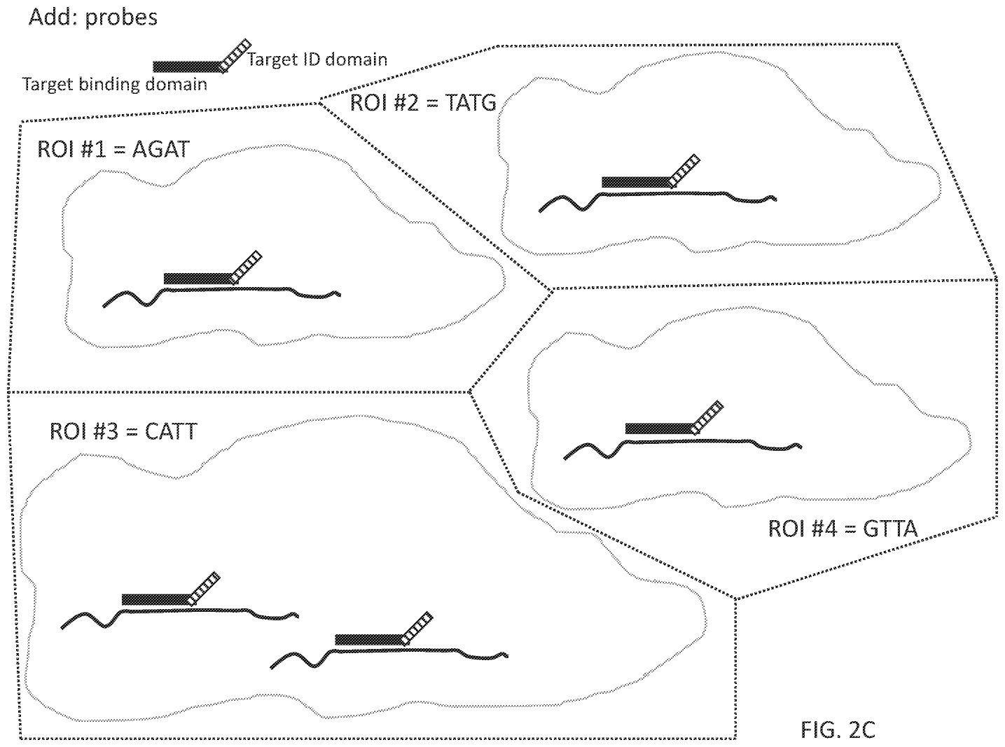

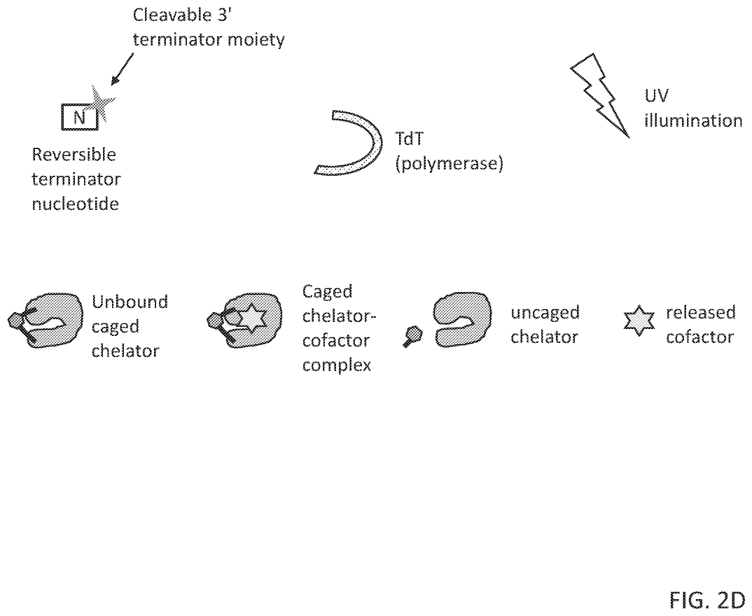

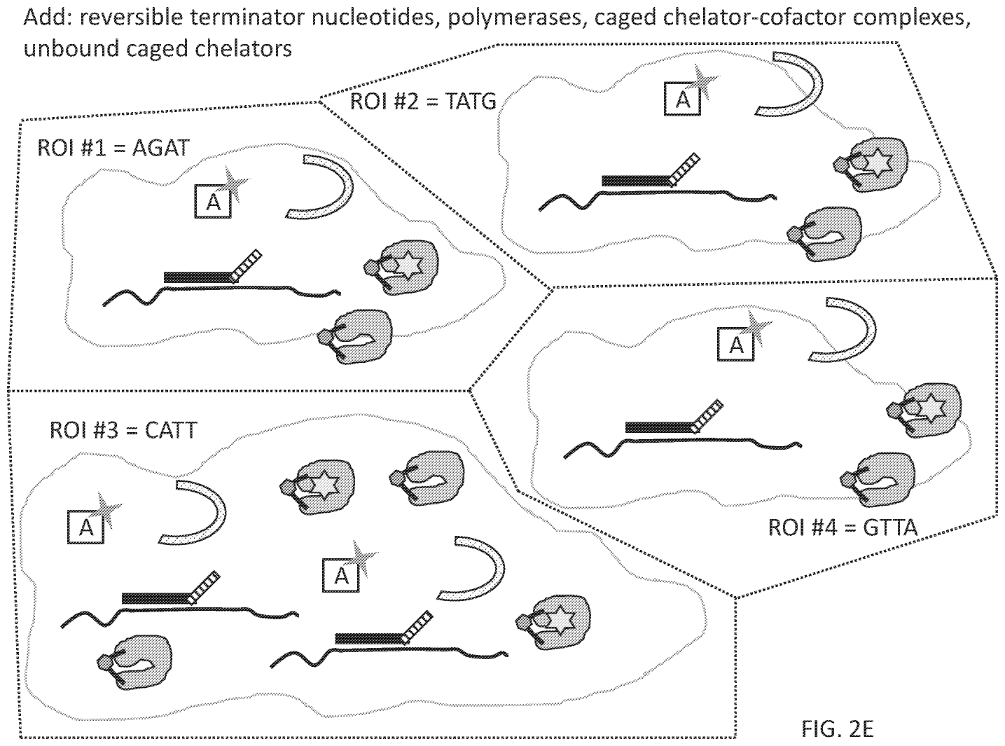

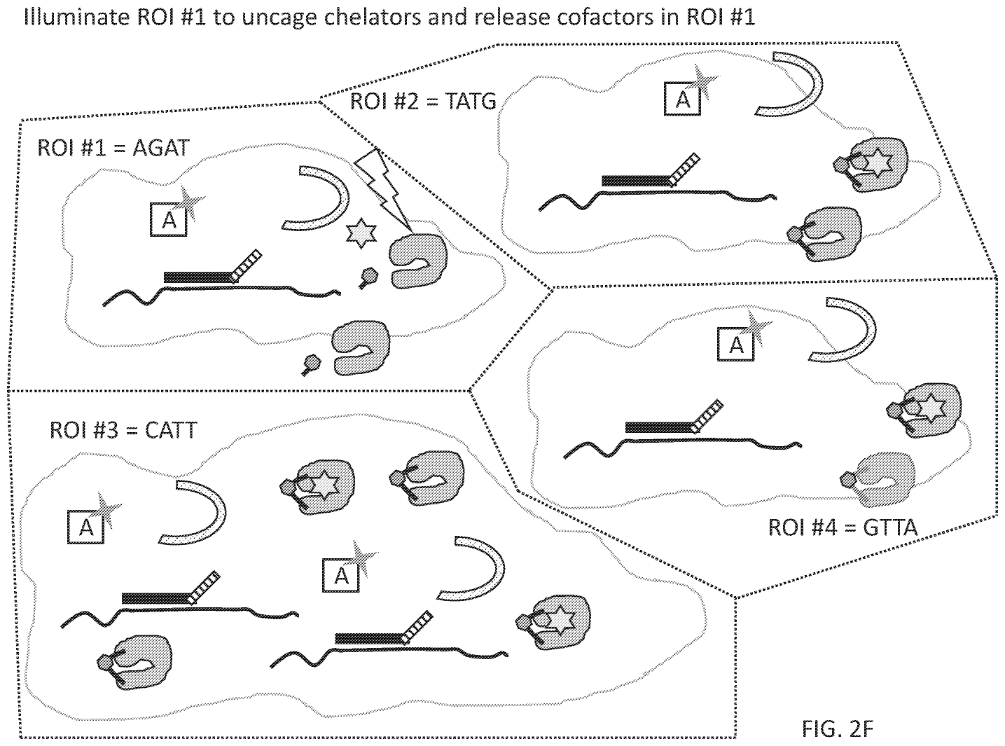

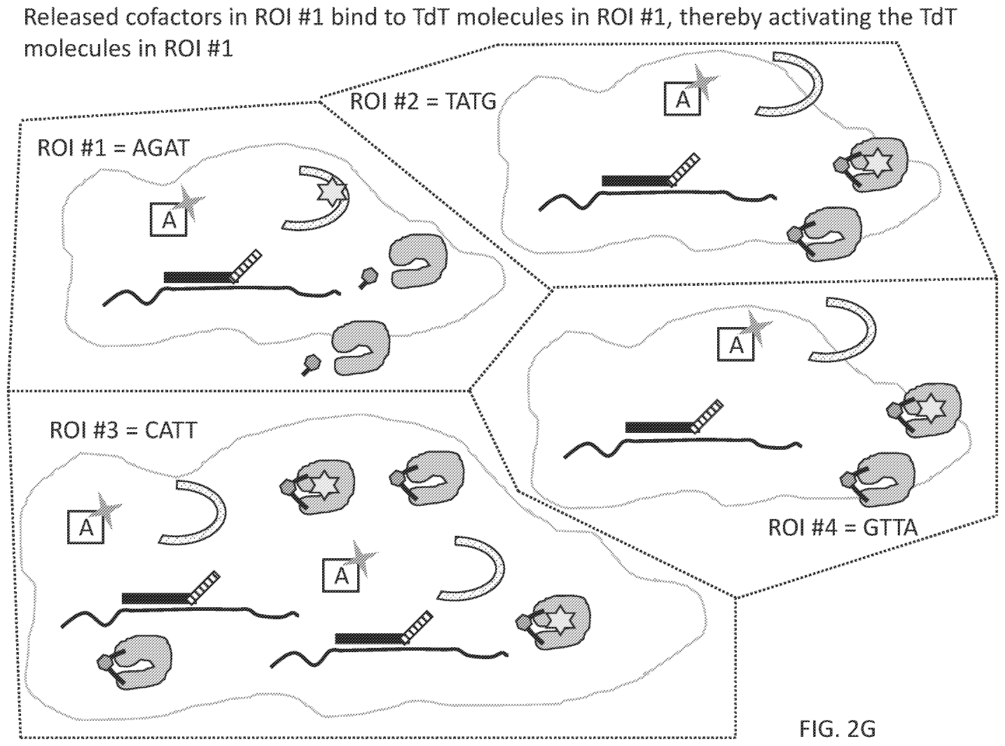

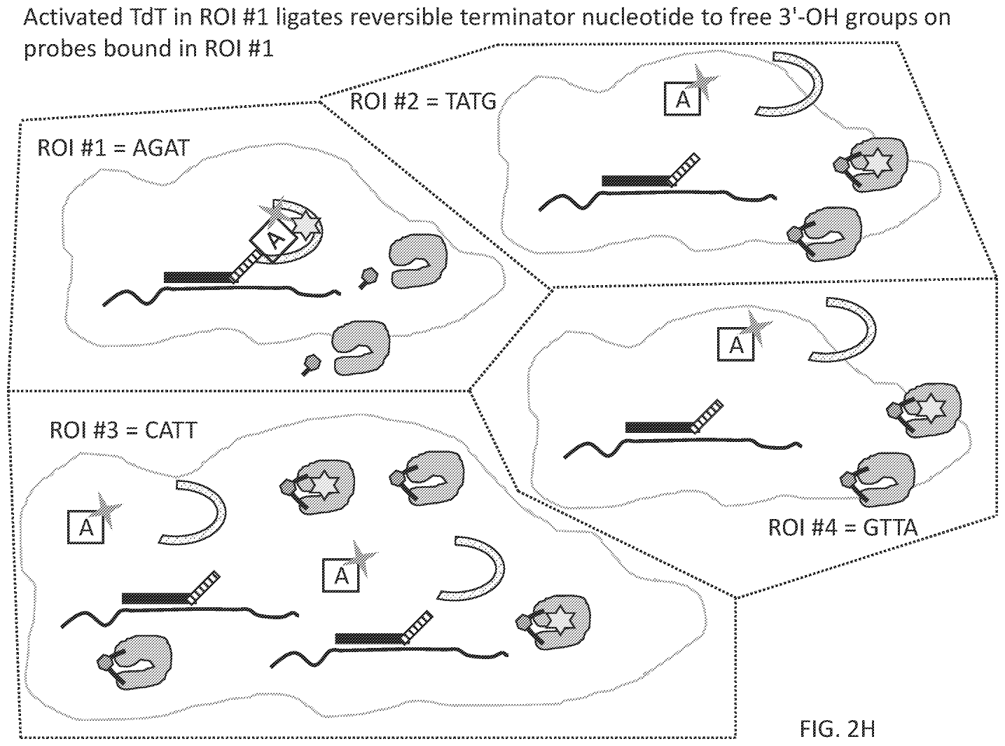

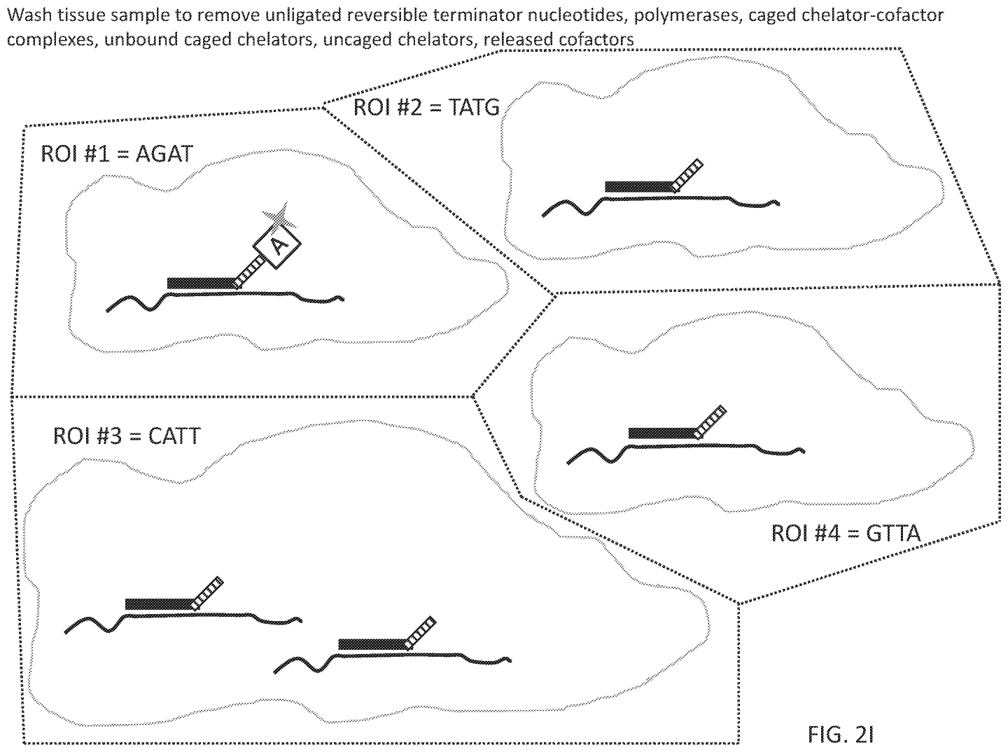

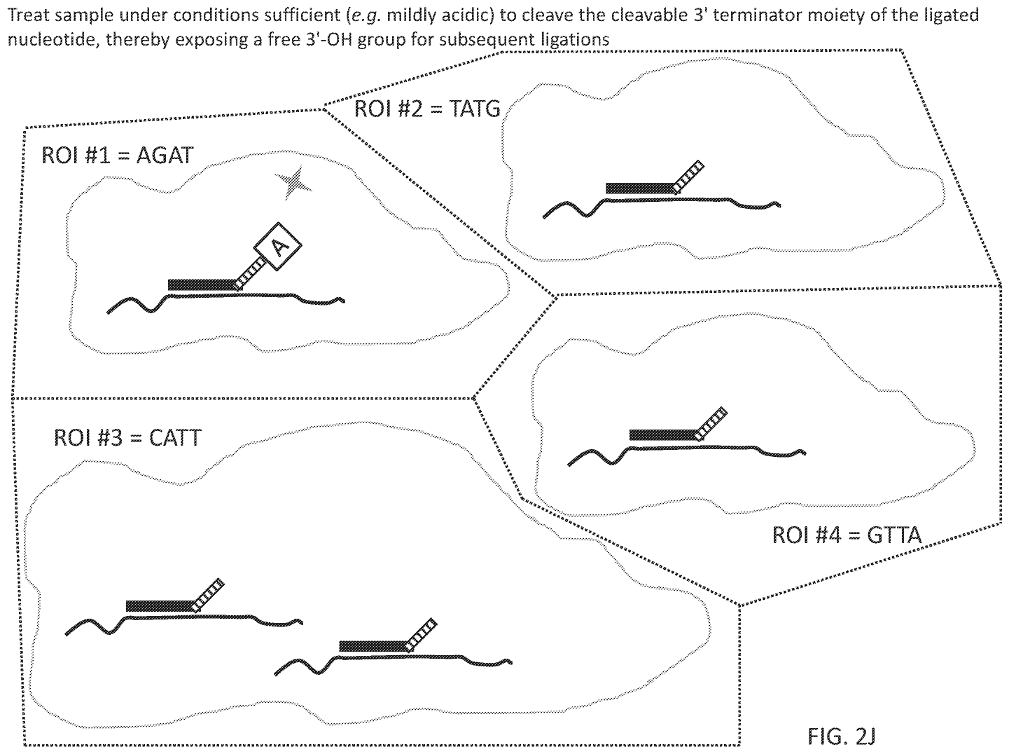

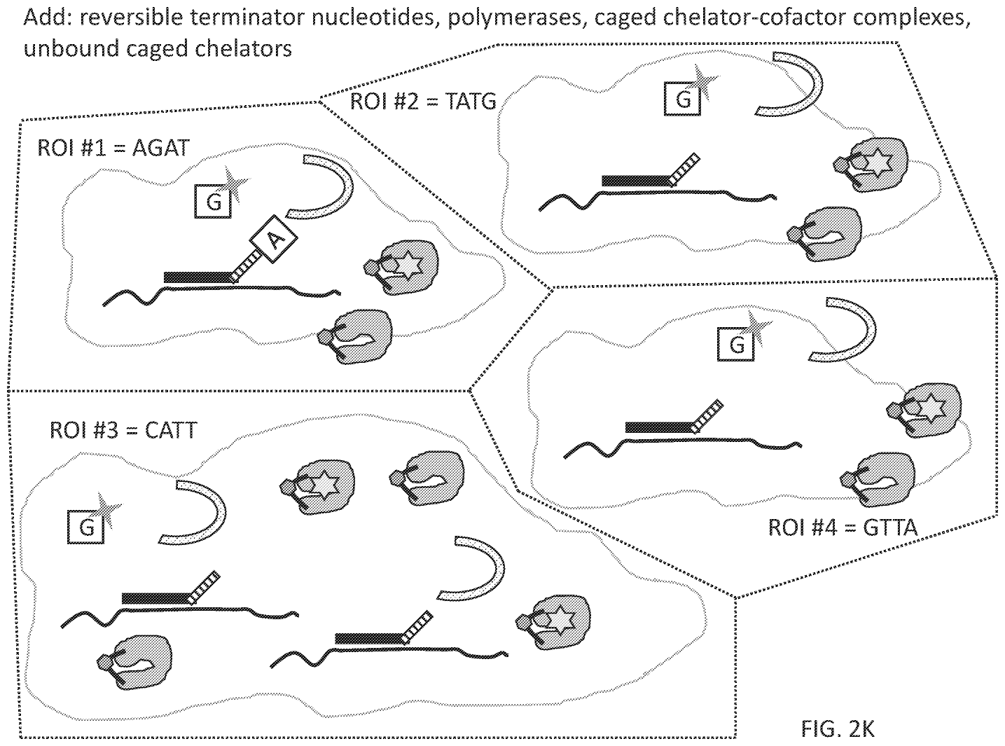

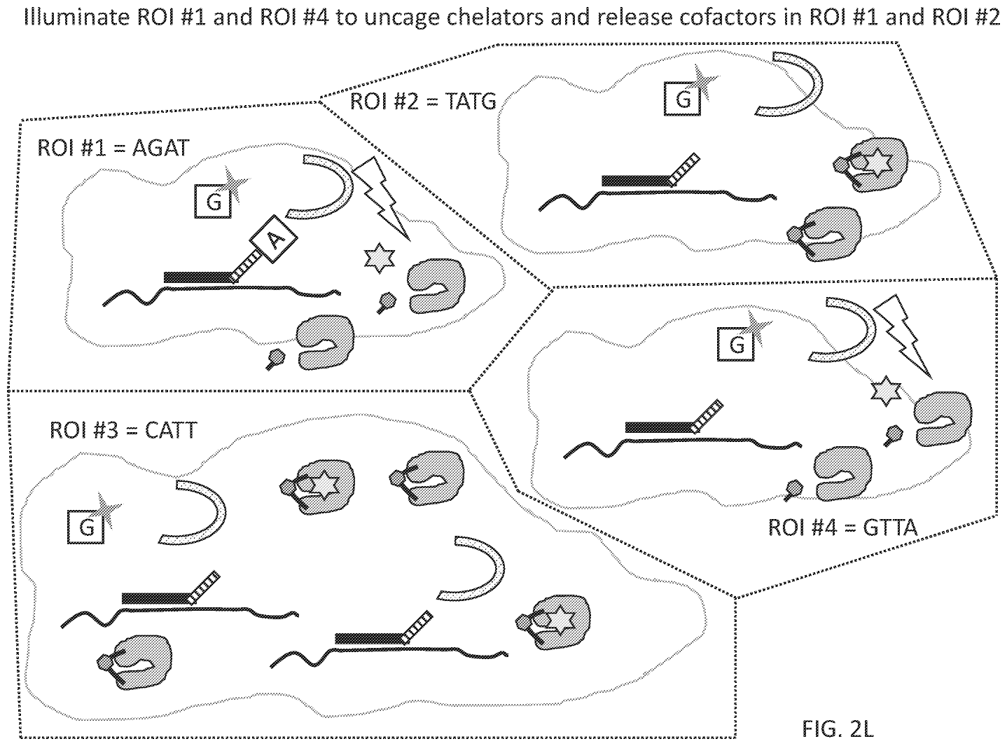

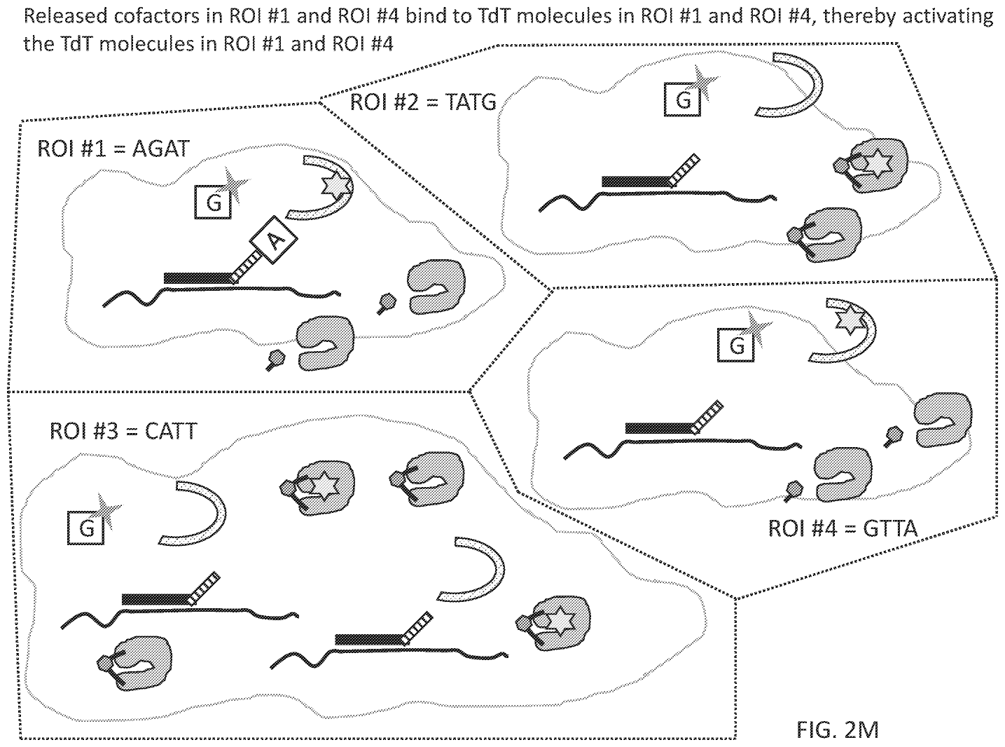

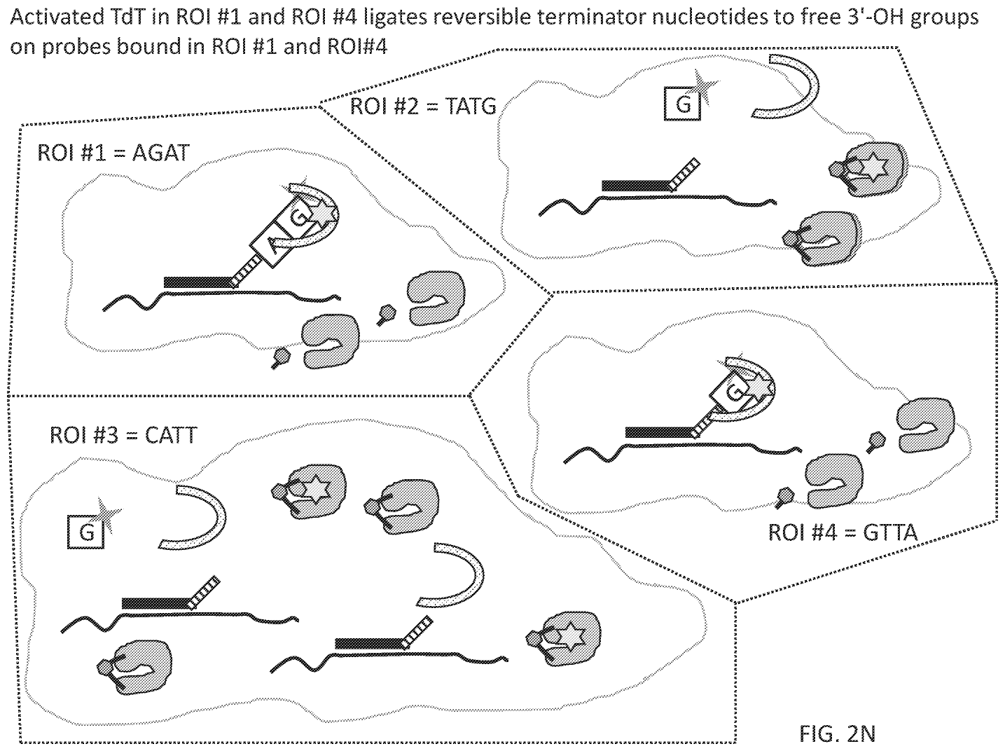

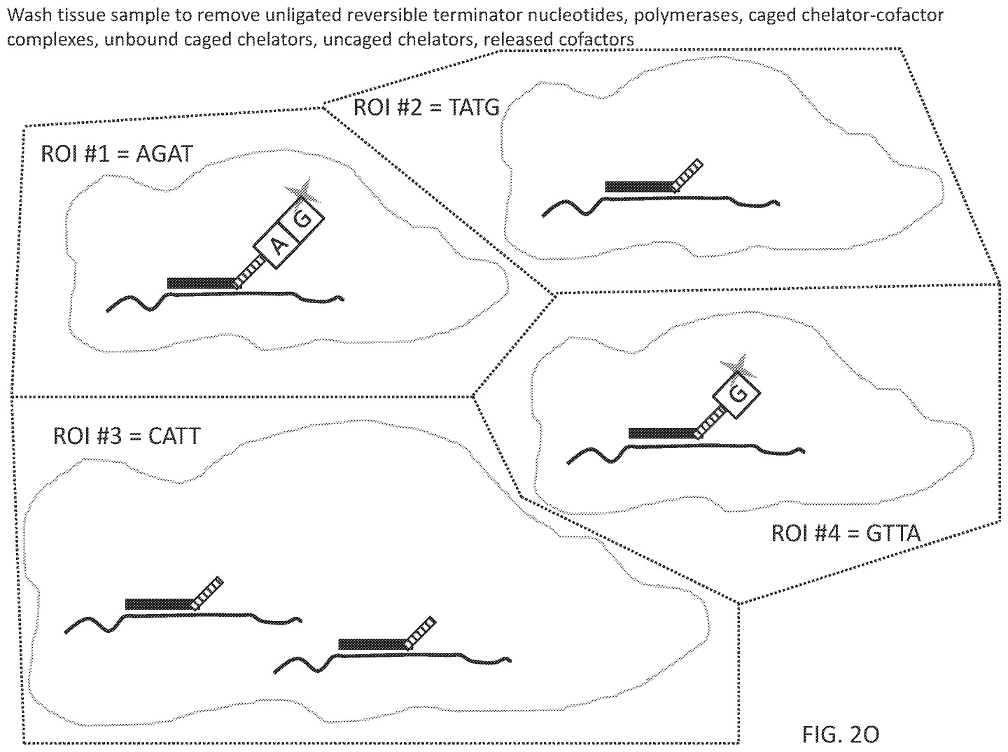

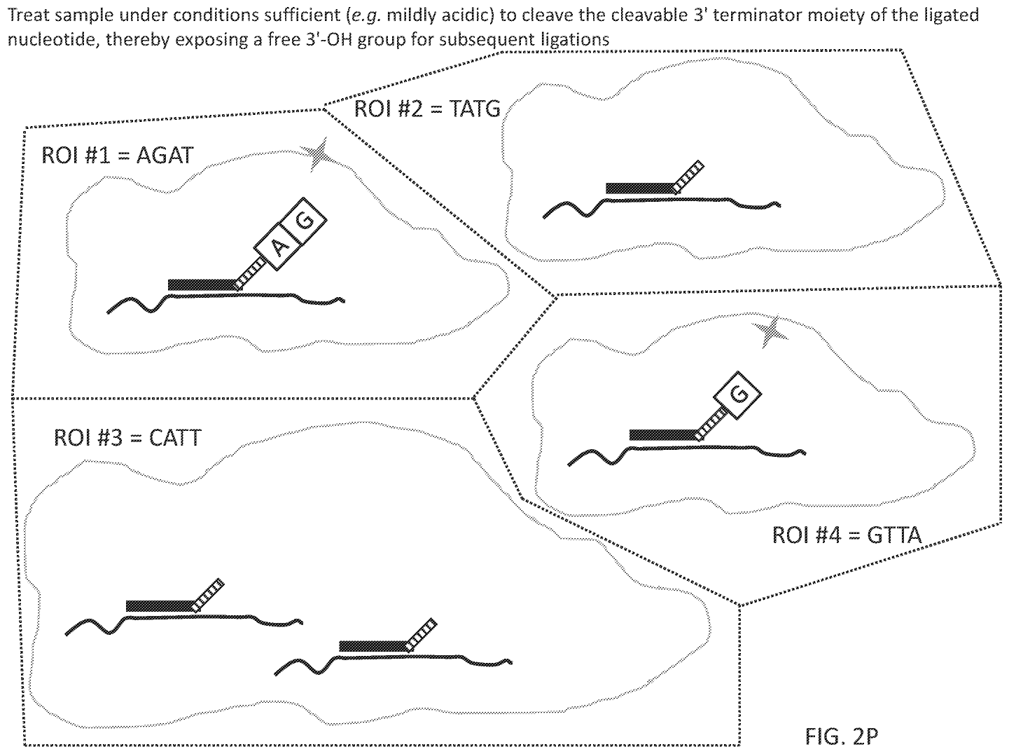

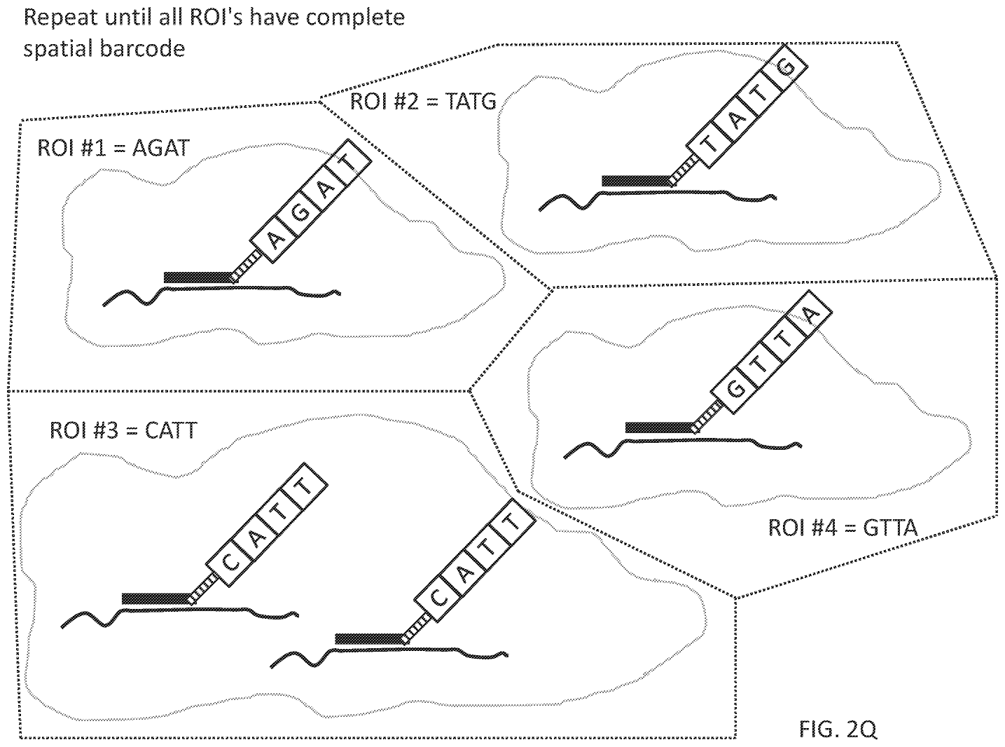

A schematic of a non-limiting example of the preceding method is shown in FIG. 2A-2Q. The method begins in FIG. 2A with a sample. In this non-limiting example, the sample comprises four cells, each cell comprising at least one copy of the specific target mRNA to be measured. Cell #1, Cell #2 and Cell #3 comprise one copy of the target mRNA and Cell #3 comprises two copies of the target mRNA.

The sample can be subdivided into regions of interest (ROI) as shown in FIG. 2B. In this non-limiting example, each ROI comprises one of the cells. Moreover, each ROI is pre-assigned a specific spatial barcode that is unique to that ROI. In this non-limiting example, each spatial barcode comprises 4 nucleotides. The spatial barcode for ROI #1 is AGAT, the spatial barcode for ROI #2 TATG, the spatial barcode for ROI #3 is CATT and the spatial barcode for ROI #4 is GTTA. As described further herein, the spatial barcode can comprise any number of nucleotides, and the four nucleotide spatial barcodes depicted in FIGS. 2A-2Q are for exemplary purposes only.

In a first step of the method, as shown in FIG. 2C, the sample is contacted with a plurality of probes, wherein the probes comprise a unique target-binding domain that binds to the target mRNA, a unique target identification domain specific for the target analyte, and a free 3′-OH moiety, as shown in the top left-hand corner of FIG. 2C. The target binding domain is specific for the target mRNA and hybridizes to each target mRNA within each cell. As described further herein, the sample may be contacted with a plurality of probes comprising any number of different species of probes wherein each species of probe comprises a unique target-binding domain that binds to one of the at least two target analytes and a unique target identification domain specific for the target analyte, and a free 3′-OH moiety. FIGS. 2A-2Q have a single species for probe for exemplary purposes only.

FIG. 2D is a schematic showing the symbols used for various different reagents/molecules in FIGS. 2E-2Q, including reversible terminator nucleotides (comprising a cleavable 3′ terminator moiety), TdT polymerase, UV illumination, unbound caged chelator, caged chelator-cofactor complex, uncaged chelator, and released cofactor.

In the second step of the method, shown in FIG. 2E, the entire tissue sample is contacted with reversible terminator nucleotides, TdT polymerases, caged chelator-cofactor complexes, and unbound caged chelators. In this non-limiting example, the first reversible terminator nucleotides to be added are Adenine nucleotides (“A”).

In the third step of the method, shown in FIG. 2F, the ROI's that require the addition of an “A” nucleotide to construct the appropriate spatial barcode are illuminated with light of sufficient wavelength to uncage the caged chelator-cofactor complexes, thereby releasing cofactors. In this non-limiting example, ROI #1 require the addition of an “A” nucleotide, so ROI #1 is illuminated in this step. In this non-limiting example, the light is UV light. The UV illumination is represented by the lightning bolt shape in FIG. 2F. The result of this UV illumination is that, within the illuminated ROI #1, the caged chelator-cofactor complexes are uncaged, thereby releasing cofactor in ROI #1. The released cofactor will then bind to TdT molecules located in ROI #1, thereby activating the TdT molecules located in ROI #1, as shown in FIG. 2G. The activated TdT molecules in ROI #I will then proceed to ligate a reversible terminator nucleotide to the free 3′-OH groups of probes bound in ROI #1, as shown in FIG. 2H. Without wishing to be bound by theory, the unbound caged chelators in any of the nonilluminated ROIs (ROI #2, ROI #3 and ROI #4) will chelate any released cofactor that diffuses from ROI #1, thereby preventing activation of TdT molecules (and therefore ligation) in the nonilluminated ROIs.

In the fourth step of the method, shown in FIG. 2I, the tissue sample is washed to remove unligated reversible terminator nucleotides, polymerases, caged chelator-cofactor complexes, unbound caged chelators, uncaged chelators, released cofactors.

In the fifth step of the method, as shown in FIG. 2J, the tissue sample is then treated under conditions sufficient to cleave the cleavable 3′ terminator moiety of the ligated reversible terminator nucleotide, thereby exposing a free 3′-OH group on the probes bound to targets in ROI #1 for subsequent rounds of ligation.

The method continues in FIG. 2K, wherein the entire tissue sample is contacted with reversible terminator nucleotides, TdT polymerases, caged chelator-cofactor complexes, and unbound caged chelators. In this non-limiting example, the second reversible terminator nucleotides to be added are Guanine nucleotides (“G”).

In the next step of the method, shown in FIG. 2L, the ROI's that require the addition of a “G” nucleotide to construct the appropriate spatial barcode are illuminated with light of sufficient wavelength to uncage the caged chelator-cofactor complexes, thereby releasing cofactors. In this non-limiting example, ROI #1 and ROI #4 require the addition of a “G” nucleotide, so ROI #1 and ROI #4 are illuminated in this step. The UV illumination is represented by the lightning bolt shape in FIG. 2L. The result of this UV illumination is that, within the illuminated ROI #1 and ROI #4, the caged chelator-cofactor complexes are uncaged, thereby releasing cofactor in ROI #1 and ROI #4. The released cofactor will then bind to TdT molecules located in ROI #1 and ROI #4, thereby activating the TdT molecules located in ROI #1 and ROI #4, as shown in FIG. 2M. The activated TdT molecules in ROI #1 and ROI #4 will then proceed to ligate a reversible terminator nucleotide to the free 3′-OH groups of probes bound in ROI #1 and ROI #4, as shown in FIG. 2N. Without wishing to be bound by theory, the unbound caged chelators in any of the nonilluminated ROIs (ROI #2 and ROI #3) will chelate any released cofactor that diffuses from ROI #1 and ROI #4, thereby preventing activation of TdT molecules (and therefore ligation) in the nonilluminated ROIs.

In the next step of the method, shown in FIG. 2O, the tissue sample is washed to remove unligated reversible terminator nucleotides, polymerases, caged chelator-cofactor complexes, unbound caged chelators, uncaged chelators, released cofactors.

In the next step of the method, as shown in FIG. 2P, the tissue sample is then treated under conditions sufficient to cleave the cleavable 3′ terminator moiety of the ligated reversible terminator nucleotide, thereby exposing a free 3′-OH group on the probes bound to targets in ROI #1 and ROI #4 for subsequent rounds of ligation.

The above-described steps can be repeated any number of times with any of the four nucleotides (dATP, dGTP, dTTP and dCTP) until all of the bound probes in each ROI have complete spatial barcodes, as shown in FIG. 2Q. After the completion of all of the spatial barcodes in each ROI, the bound probes can be washed from the sample, collected, and then sequenced. By sequencing the spatial barcode for each collected probe, the ROI from which that specific probe originated can be identified, allowing for the quantification of the abundance of each target mRNA within each ROI, thereby producing a spatially-resolved profile of the abundance of the target mRNA.

The order of nucleotide addition (i.e. A, then T then G) in FIGS. 2A-2Q are for exemplary purposes only. In methods of the present disclosure, nucleotides can be added to the spatial barcodes in any order and/or combination to synthesize the appropriate spatial barcodes for each ROI.

In some aspects of the methods of the present disclosure, a polymerase can be terminal deoxynucleotidyl transferase (TdT) or a portion thereof. In some aspects, the TdT can be Mus musculus TdT. In some aspects, the TdT can be the short isoform of Mus musculus TdT, which has the amino acid sequence:

(SEQ ID NO: 1)
MDPLQAVHLGPRKKRPRQLGTPVASTPYDIRFRDLVLFILEKKMGTTRRA
FLMELARRKGFRVENELSDSVTHIVAENNSGSDVLEWLQLQNIKASSELE
LLDISWLIECMGAGKPVEMMGRHQLVVNRNSSPSPVPGSQNVPAPAVKKI
SQYACQRRTTLNNYNQLFTDALDILAENDELRENEGSCLAFMRASSVLKS
LPFPITSMKDTEGIPCLGDKVKSIIEGIIEDGESSEAKAVLNDERYKSFK
LFTSVFGVGLKTAEKWFRMGFRTLSKIQSDKSLRFTQMQKAGFLYYEDLV
SCVNRPEAEAVSMLVKEAVVTFLPDALVTMTGGFRRGKMTGHDVDFLITS
PEATEDEEQQLLHKVTDFWKQQGLLLYCDILESTFEKFKQPSRKVDALDH
FQKCFLILKLDHGRVHSEKSGQQEGKGWKAIRVDLVMCPYDRRAFALLGW
TGSRQFERDLRRYATHERKMMLDNHALYDRTKRVFLEAESEEEIFAHLGL
DYIEPWERNA

In some aspects, a polymerase can comprise residues 132-510 of the short isoform of Mus musculus TdT. The amino acid sequence of residues 132-510 of the short isoform Mus musculus TdT is:

(SEQ ID NO: 2)
SPSPVPGSQNVPAPAVKKISQYACQRRTTLNNYNQLFTDALDILAENDEL
RENEGSCLAFMRASSVLKSLPFPITSMKDTEGIPCLGDKVKSIIEGIIED
GESSEAKAVLNDERYKSFKLFTSVFGVGLKTAEKWFRMGFRTLSKIQSDK
SLRFTQMQKAGFLYYEDLVSCVNRPEAEAVSMLVKEAVVTFLPDALVTMT
GGFRRGKMTGHDVDFLITSPEATEDEEQQLLHKVTDFWKQQGLLLYCDIL
ESTFEKFKQPSRKVDALDHFQKCFLILKLDHGRVHSEKSGQQEGKGWKAI
RVDLVMCPYDRRAFALLGWTGSRQFERDLRRYATHERKMMLDNHALYDRT
KRVFLEAESEEEIFAHLGLDYIEPWERNA

In some aspects, a polymerase can comprise residues 132-510 of the short isoform of Mus musculus TdT, wherein all exposed native cysteine residues have been removed, such that the cysteine at position 188 is substituted by Alanine, the cysteine at position 216 is substituted by Serine, the cysteine at position 302 is substituted by Alanine, the cysteine at position 378 is substituted by Alanine and the cysteine at position 438 is substituted by Serine. This sequence, herein referred to as “exposed Cys-less 132-510 Mus musculus TdT” has the amino acid sequence:

(SEQ ID NO: 3)
SPSPVPGSQNVPAPAVKKISQYACQRRTTLNNYNQLFTDALDILAENDEL
RENEGSALAFMRASSVLKSLPFPITSMKDTEGIPSLGDKVKSIIEGIIED
GESSEAKAVLNDERYKSFKLFTSVFGVGLKTAEKWFRMGFRTLSKIQSDK
SLRFTQMQKAGFLYYEDLVSAVNRPEAEAVSMLVKEAVVTFLPDALVTMT
GGFRRGKMTGHDVDFLITSPEATEDEEQQLLHKVTDFWKQQGLLLYADIL
ESTFEKFKQPSRKVDALDHFQKCFLILKLDHGRVHSEKSGQQEGKGWKAI
RVDLVMSPYDRRAFALLGWTGSRQFERDLRRYATHERKMMLDNHALYDRT
KRVFLEAESEEEIFAHLGLDYIEPWERNA

In some aspects, a polymerase can comprise the sequence of exposed Cys-less 132-510 Mus musculus TdT, wherein the Glutamic Acid at position 180 is replaced with a cysteine residue. This sequence, herein referred to as MmTdT180 has the amino acid sequence:

(SEQ ID NO: 4)
SPSPVPGSQNVPAPAVKKISQYACQRRTTLNNYNQLFTDALDILAENDCL
RENEGSALAFMRASSVLKSLPFPITSMKDTEGIPSLGDKVKSIIEGIIED
GESSEAKAVLNDERYKSFKLFTSVFGVGLKTAEKWFRMGFRTLSKIQSDK
SLRFTQMQKAGFLYYEDLVSAVNRPEAEAVSMLVKEAVVTFLPDALVTMT
GGFRRGKMTGHDVDFLITSPEATEDEEQQLLHKVTDFWKQQGLLLYADIL
ESTFEKFKQPSRKVDALDHFQKCFLILKLDHGRVHSEKSGQQEGKGWKAI
RVDLVMSPYDRRAFALLGWTGSRQFERDLRRYATHERKMMLDNHALYDRT
KRVFLEAESEEEIFAHLGLDYIEPWERNA

In some aspects, a polymerase can comprise the sequence of exposed Cys-less 132-510 Mus musculus TdT, wherein the Alanine at position 188 is replaced with a cysteine residue. This sequence, herein referred to as MmTdT188 has the amino acid sequence:

(SEQ ID NO: 5)
SPSPVPGSQNVPAPAVKKISQYACQRRTTLNNYNQLFTDALDILAENDEL
RENEGSCLAFMRASSVLKSLPFPITSMKDTEGIPSLGDKVKSIIEGIIED
GESSEAKAVLNDERYKSFKLFTSVFGVGLKTAEKWFRMGFRTLSKIQSDK
SLRFTQMQKAGFLYYEDLVSAVNRPEAEAVSMLVKEAVVTFLPDALVTMT
GGFRRGKMTGHDVDFLITSPEATEDEEQQLLHKVTDFWKQQGLLLYADIL
ESTFEKFKQPSRKVDALDHFQKCFLILKLDHGRVHSEKSGQQEGKGWKAI
RVDLVMSPYDRRAFALLGWTGSRQFERDLRRYATHERKMMLDNHALYDRT
KRVFLEAESEEEIFAHLGLDYIEPWERNA

In some aspects, a polymerase can comprise the sequence of exposed Cys-less 132-510 Mus musculus TdT, wherein the Threonine at position 253 is replaced with a cysteine residue. This sequence, herein referred to as MmTdT253 has the amino acid sequence:

(SEQ ID NO: 6)
SPSPVPGSQNVPAPAVKKISQYACQRRTTLNNYNQLFTDALDILAENDEL
RENEGSALAFMRASSVLKSLPFPITSMKDTEGIPSLGDKVKSIIEGIIED
GESSEAKAVLNDERYKSFKLFCSVFGVGLKTAEKWFRMGFRTLSKIQSDK
SLRFTQMQKAGFLYYEDLVSAVNRPEAEAVSMLVKEAVVTFLPDALVTMT
GGFRRGKMTGHDVDFLITSPEATEDEEQQLLHKVTDFWKQQGLLLYADIL
ESTFEKFKQPSRKVDALDHFQKCFLILKLDHGRVHSEKSGQQEGKGWKAI
RVDLVMSPYDRRAFALLGWTGSRQFERDLRRYATHERKMMLDNHALYDRT
KRVFLEAESEEEIFAHLGLDYIEPWERNA

In some aspects, a polymerase can comprise the sequence of exposed Cys-less 132-510 Mus musculus TdT, wherein the Alanine at position 302 is replaced with a cysteine residue. This sequence, herein referred to as MmTdT253 has the amino acid sequence:

(SEQ ID NO: 7)
SPSPVPGSQNVPAPAVKKISQYACQRRTTLNNYNQLFTDALDILAENDEL
RENEGSALAFMRASSVLKSLPFPITSMKDTEGIPSLGDKVKSIIEGIIED
GESSEAKAVLNDERYKSFKLFTSVFGVGLKTAEKWFRMGFRTLSKIQSDK
SLRFTQMQKAGFLYYEDLVSCVNRPEAEAVSMLVKEAVVTFLPDALVTMT
GGFRRGKMTGHDVDFLITSPEATEDEEQQLLHKVTDFWKQQGLLLYADIL
ESTFEKFKQPSRKVDALDHFQKCFLILKLDHGRVHSEKSGQQEGKGWKAI
RVDLVMSPYDRRAFALLGWTGSRQFERDLRRYATHERKMMLDNHALYDRT
KRVFLEAESEEEIFAHLGLDYIEPWERNA

In some aspects of the methods of the present disclosure, a cleavable 3′ terminator moiety can be a 3′-ONH₂ group.

In some aspects of the methods of the present disclosure, a caged-chelator can be a caged divalent cation chelator. In some aspects of the methods of the present disclosure, a caged-chelator can be 1-(4,5-Dimethoxy-2-Nitrophenyl)-1,2-Diaminoethane-N,N,N′,N′-Tetraacetic Acid (DMNP-EDTA).

In some aspects of the methods of the present disclosure, a cofactor can be a divalent metal cofactor. In some aspects of the methods of the present disclosure, a cofactor can be Co²⁺, Mg²⁺, Mn²⁺ Ca²⁺, Cd²⁺, Zn²⁺ or Fe²⁺. In some aspects of the methods of the present disclosure, a cofactor can be Co²⁺.

In some aspects of the methods of the present disclosure, contacting a tissue sample with a cofactor can comprise contacting the tissue sample with a salt form of the cofactor.

In some aspects of the methods of the present disclosure, light sufficient to uncage the caged chelator-cofactor complex can be UV light.

In some aspects of the methods of the present disclosure, treating a tissue sample under conditions sufficient to cleave a 3′ terminator moiety of an at least one reversible terminator nucleotide can comprise treating a tissue sample under acidic conditions. In some aspects, treating a tissue sample under acidic conditions comprises contacting the tissue sample with a solution with a pH of about 5.5.

In some aspects, the methods of the present disclosure can be used to produce a spatially-resolved profile of the abundance of any number of target analytes in at least about 10, or at least about 100, or at least about 1,000, or at least about 10,000, or at least about 100,000, or at least about 1,000,000, or at least about 107, or at least about 108, or at least about 109, or at least about 1010, or at least about 1011, or at least about 1012, or at least about 1013, or at least about 1014, or at least about 1015, or more locations in a tissue sample.

In some aspects, the methods of the present disclosure can be used to produce a spatially-resolved profile of the abundance of at least about three, or at least about four, or at least about five, or at least about six, or at least about seven, or at least about eight, or at least about nine, or at least about ten, or at least about 20, or at least about 30, or at least about 40, or at least about 50, or at least about 60, or at least about 70, or at least about 80, or at least about 90, or at least about 100, or at least about 250, or at least about 500, or at least about 750, or at least about 1000, or at least about 5000, or at least about 10,000, or at least about 100,000 target analytes in any number of locations in a tissue sample

In some aspects of the methods of the present disclosure, contacting a tissue sample with a solution comprising at least two species of probes can comprise contacting a tissue sample with at least about three, or at least about four, or at least about five, or at least about six, or at least about seven, or at least about eight, or at least about nine, or at least about ten, or at least about 20, or at least about 30, or at least about 40, or at least about 50, or at least about 60, or at least about 70, or at least about 80, or at least about 90, or at least about 100, or at least about 250, or at least about 500, or at least about 750, or at least about 1000, or at least about 5000, or at least about 10,000, or at least about 100,000 species of probes, the probes comprising a target-binding domain and a target identification domain, wherein each species of probe comprises a unique target-binding domain that binds to one of the at least two target analytes and a unique target identification domain specific for the target analyte, and a free 3′-OH moiety.

As used herein, the term “spatially detecting” is used in its broadest sense to refer to the identification of the presence of a specific target analyte within a specific region of interest in a sample. Spatially detecting can comprise quantifying the amount of a specific target analyte present within a specific region of interest in a sample. Spatially detecting can further comprise quantifying the relative amount of a first target analyte within a specific region of interest in a sample as compared to the amount of at least a second target analyte within a specific region of interest in a sample. Spatially detecting can also comprise quantifying the relative amount of a specific target analyte within a first region of interest in a sample compared to the amount of the same target analyte in at least a second region of interest in the same sample or different sample.

### Target Analyte

In some aspects of the methods and compositions of the present disclosure, a target analyte can be any molecule within a sample that is to be spatially detected Target analytes include, but are not limited to, nucleic acid molecules and protein molecules. When the target analyte is a protein, the protein can be referred to as a target protein. When the target analyte is a nucleic acid, the nucleic acid can be referred to as a target nucleic acid. Target nucleic acids can include, but are not limited to, mRNA molecules, microRNA (miRNA) molecules, tRNA molecules, rRNA molecules, gDNA or any other nucleic acid present within a sample.

In some aspects of the methods of the present disclosure, a target analyte is a target nucleic acid. A target nucleic acid can be DNA or RNA. A target nucleic acid can be a messenger RNA (mRNA). A target nucleic acid can be a microRNA (miRNA).

### Probes

In some aspects of the methods of the present disclosure, a probe can be a polynucleotide. In some aspects, a probe can comprise DNA, RNA or a combination of DNA and RNA. In some aspects, a probe can comprise DNA. In some aspects, a probe can be a single-stranded polynucleotide. In some aspects, a probe can be a double-stranded polynucleotide. In some aspects, a probe can be a partially single-stranded polynucleotide. In some aspects, a probe can be a partially double-stranded polynucleotide.

In some aspects of the methods of the present disclosure, a probe can comprise a target-binding domain.

The target binding domain can comprise a series of nucleotides (e.g. is a polynucleotide). The target binding domain can comprise DNA, RNA, or a combination thereof. In some aspects, the target binding domain comprises DNA. In some aspects, the target-binding domain is a single-stranded polynucleotide.

In aspects wherein the target binding domain is a polynucleotide, the target binding domain can bind directly or indirectly to a target nucleic acid. A target binding domain can bind directly to a target nucleic acid by hybridizing to a portion of the target nucleic acid that is complementary to the target binding domain of the sequencing probe.

In some aspects of the methods of the present disclosure, that target-binding domain of a probe can indirectly hybridize to a target nucleic acid present in a sample (via an intermediary oligonucleotide).

The target binding domain of the sequencing probe can be designed to control the likelihood of sequencing probe hybridization and/or de-hybridization and the rates at which these occur. Generally, the lower a probe's Tm, the faster and more likely that the probe will de-hybridize to/from a target nucleic acid. Thus, use of lower Tm probes will decrease the number of probes bound to a target nucleic acid.

The length of a target binding domain, in part, affects the likelihood of a probe hybridizing and remaining hybridized to a target nucleic acid. Generally, the longer (greater number of nucleotides) a target binding domain is, the less likely that a complementary sequence will be present in the target nucleotide. Conversely, the shorter a target binding domain is, the more likely that a complementary sequence will be present in the target nucleotide.

A target binding domain can be any number of nucleotides in length. In some aspects, a target binding domain can be at least about two, or at least about three, or at least about four, or at least about five, or at least about six, or at least about seven, or at least about eight, or at least about nine, or at least about ten, or at least about 11, or at least about 12, or at least about 13, or at least about 14, or at least about 15, or at least about 16, or at least about 17, or at least about 18, or at least about 19, or at least about 20, or at least about 21, or at least about 22, or at least about 23, or at least about 24, or at least about 25, or at least about 26, or at least about 27, or at least about 28, or at least about 29, or at least about 30, or at least about 31, or at least about 32, or at least about 33, or at least about 34, or at least about 35, or at least about 36, or at least about 37, or at least about 38, or at least about 39, or at least about 40, or at least about 41, or at least about 42, or at least about 43, or at least about 44, or at least about 45, or at least about 46, or at least about 47, or at least about 48, or at least about 49, or at least about 50 or more nucleotides in length.

Probes of the present disclosure can be used for spatially detecting a target nucleic acid. In this aspect, the target binding domain can be a target nucleic acid-binding region. The target nucleic acid-binding region is preferably at least 15 nucleotides in length, and more preferably is at least 20 nucleotides in length. In specific aspects, the target nucleic acid-binding region is approximately 10 to 500, 20 to 400, 25, 30 to 300, 35, 40 to 200, or 50 to 100 nucleotides in length. Probes and methods for binding and identifying a target nucleic acid have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety.

The target binding domain can comprise at least one natural base. The target binding domain can comprise no natural bases. The target binding domain can comprise at least one modified nucleotide or nucleic acid analog. The target binding domain can comprise no modified nucleotides or nucleic acid analogs. The target binding domain can comprise at least one universal base. The target binding domain can comprise no universal bases. The target binding domain can comprise at least one degenerate base. The target binding domain can comprise no degenerate bases.

The target domain can comprise any combination natural bases (e.g. 0, 1, 2, 3, 4, 5, 6, 7, 8, 9, 10, or more natural bases), modified nucleotides or nucleic acid analogs (e.g. 0, 1, 2, 3, 4, 5, 6, 7, 8, 9, 10, or more modified nucleotides or nucleic acid analogs), universal bases (e.g. 0, 1, 2, 3, 4, 5, 6, 7, 8, 9, 10, or more universal bases), or degenerate bases (e.g. 0, 1, 2, 3, 4, 5, 6, 7, 8, 9, 10, or more degenerative bases). When present in a combination, the natural bases, modified nucleotides or nucleic acid analogs, universal bases and degenerate bases of a particular target binding domain can be arranged in any order.

The terms “modified nucleotides” or “nucleic acid analogues” include, but are not limited to, locked nucleic acids (LNA), bridged nucleic acids (BNA), propyne-modified nucleic acids, zip nucleic acids (ZNA®), isoguanine, isocytosine 6-amino-1-(4-hydroxy-5-hydroxy methyl-tetrahydro-furan-2-yl)-1,5-dihydro-pyrazolo[3,4-d]pyrimidin-4-one (PPG) and 2′-modified nucleic acids such as 2′-O-methyl nucleic acids. The target binding domain can include zero to six (e.g. 0, 1, 2, 3, 4, 5 or 6) modified nucleotides or nucleic acid analogues. Preferably, the modified nucleotides or nucleic acid analogues are locked nucleic acids (LNAs).

The term “locked nucleic acids (LNA)” as used herein includes, but is not limited to, a modified RNA nucleotide in which the ribose moiety comprises a methylene bridge connecting the 2′ oxygen and the 4′ carbon. This methylene bridge locks the ribose in the 3′-endo confirmation, also known as the north confirmation, that is found in A-form RNA duplexes. The term inaccessible RNA can be used interchangeably with LNA. The term “bridged nucleic acids (BNA)” as used herein includes, but is not limited to, modified RNA molecules that comprise a five-membered or six-membered bridged structure with a fixed 3′-endo confirmation, also known as the north confirmation. The bridged structure connects the 2′ oxygen of the ribose to the 4′ carbon of the ribose. Various different bridge structures are possible containing carbon, nitrogen, and hydrogen atoms. The term “propyne-modified nucleic acids” as used herein includes, but is not limited to, pyrimidines, namely cytosine and thymine/uracil, that comprise a propyne modification at the C5 position of the nucleic acid base. The term “zip nucleic acids (ZNA®)” as used herein includes, but is not limited to, oligonucleotides that are conjugated with cationic spermine moieties.

The term “universal base” as used herein includes, but is not limited to, a nucleotide base does not follow Watson-Crick base pair rules but rather can bind to any of the four canonical bases (A, T/U, C, G) located on the target nucleic acid. The term “degenerate base” as used herein includes, but is not limited to, a nucleotide base that does not follow Watson-Crick base pair rules but rather can bind to at least two of the four canonical bases A, T/U, C, G), but not all four. A degenerate base can also be termed a Wobble base; these terms are used interchangeably herein.

In some aspects, the term “degenerate base” can also be used to refer to a specific position on a nucleic acid molecule (e.g. an amplification primer, a probe, etc.) that, in the context of a plurality of the nucleic acid molecules, can independently be one or more different nucleotides on different nucleic acid molecules within said plurality. In a non-limiting example, a plurality of 4-nucleotide long nucleic acid molecules may have a degenerate base at the fourth nucleotide position, wherein the degenerate base can be either A, C or G, and wherein the nucleotides at positions 1, 2 and 3 are the same between different nucleic acid molecules. That is, within said plurality, there may be nucleic acid molecules with A at the fourth position, nucleic acid molecules with C at the fourth position, and nucleic acid molecules with G at the fourth position, while all of the nucleic acid molecules are identical at positions 1, 2 and 3.

In some aspects of the methods of the present disclosure, a probe can comprise a target identification domain.

A target identification domain is a nucleic acid molecule that identifies the target analyte bound to the target binding domain of a probe. The target identification domain comprises a unique nucleic acid sequence that identifies the target analyte bound to the target binding domain of the probe. In a non-limiting example, a probe with a target binding domain that binds to the protein P53 comprises a target identification domain with a unique nucleic acid sequence that corresponds to P53, while a probe with a target binding domain that binds to the protein P97 comprises a target identification domain with a unique nucleic acid sequence that corresponds to P97.

A target identification domain can be any number of nucleotides in length. In some aspects, a target identification domain can be at least about 12 nucleotides in length. In some aspects, a target identification domain be at least about 7 to at least about 17 nucleotides in length. In some aspects, a target identification domain be at least about 2 to at least about 22 nucleotides in length. In some aspects, a target identification domain can be at least about 2, or at least about 3, or at least about 4, or at least about 5, or at least about 6, or at least about 7, or at least about 8, or at least about 9, or at least about 10, or at least about 11, or at least about 12, or at least about 13, or at least about 14, or at least about 15, or at least about 16, or at least about 17, or at least about 18, or at least about 19, or at least about 20, or at least about 21, or at least about 22, or at least about 23, or at least about 24, or at least about 25, or at least about 26, or at least about 27, or at least about 28, or at least about 29, or at least about 30, or at least about 31, or at least about 32, or at least about 33, or at least about 34, or at least about 35, or at least about 36, or at least about 37, or at least about 38, or at least about 39 or at least about 40 nucleotides in length.

In the context of a probe, a target identification domain is a polynucleotide that comprises a nucleic acid sequence that identifies the target analyte bound to the target binding domain of that probe. That is to say, the target identification domain comprises a specific nucleic acid sequence that is a priori assigned to the specific target analyte bound to the target binding to which the target identification domain is attached. In a non-limiting example, a probe designated as “probe X” designed to spatially detect “target analyte A” comprises a target binding domain designated “target binding domain X” linked to a target identification domain “target identification domain X”. Target binding domain X binds to target analyte X′ and target identification domain X comprises a nucleic acid sequence, designated as “nucleic acid sequence X”, which corresponds to target analyte X. Thus, if a skilled artisan practicing the methods of the present disclosure were to collect probes a region of interest in sample and obtain nucleic acid sequence X after sequencing, the skilled artisan would understand that to indicate that target analyte X was present in that region of interest. The amount, or number of sequencing reads, of nucleic acid sequence X can be used to determine the quantify, in absolute or relative terms, the amount of target analyte X within the region of interest.

In some aspects of the methods of the present disclosure, a probe can comprise a unique molecular identifier (UMI).

In some aspects, a unique molecular identifier can be at least about 9 nucleotides to at least about 19 nucleotides in length. In some aspects, a unique molecular identifier can be at least about 4 nucleotides to at least about 24 nucleotides in length. In some aspects, a unique molecular identifier can be about 14 nucleotides in length. The terms unique molecular identifier and random molecular tags are used interchangeably herein. Using methods known in that art, unique molecular identifiers can be used to correct for biases in amplification prior to sequencing.

In some aspects of the methods and compositions of the present disclosure, a molecular identifier can comprise at least about 5, or at least about 10 nucleotides, or at least about 15, or at least about 20, or at least about 25, or at least about 30, or at least about 35, or at least about 40, or at least about 45, or at least about 50 nucleotides.

In some aspects of the methods of the present disclosure, a probe can comprise an amplification primer binding site. As used herein, the term “amplification primer binding site” is used in its broadest sense to refer to a nucleic acid sequence that is complementary to, or at least partially complementary to at least one amplification primer, wherein the amplification primer is a short single-stranded or partially single-stranded oligonucleotide that is sufficient to prime DNA and/or RNA synthesis, for example, by PCR.

In some aspects of the methods of the present disclosure, an amplification primer binding site can comprise at least about 5, at least about 10, at least about 15, at least about 20, at least about 25, at least about 30, at least about 35, at least about 40, at least about 45, at least about 50, at least about 55, at least about 60, at least about 65 or at least about 70 nucleotides.

In some aspects of the methods of the present disclosure, a probe can comprise a constant region. In some aspects a constant region can comprise a specific nucleic acid sequence that is the same for each location of the tissue sample.

In some aspects of the methods of the present disclosure, a constant region can be at least about 1, or at least about 2, or at least about 3, or at least about 4, or at least about 5, or at least about 6, or at least about 7, or at least about 8, or at least about 9, or at least about 10, or at least about 11, or at least about 12, or at least about 13, or at least about 14, or at least about 15, or at least about 16, or at least about 17, or at least about 18, or at least about 19, or at least about 20, or at least about 21, or at least about 22, or at least about 23, or at least about 24, or at least about 25, or at least about 26, or at least about 27, or at least about 28, or at least about 29, or at least about 30 nucleotides in length.

In some aspects of the methods of the present disclosure, a constant region can be located between a target identification domain and a free 3′-OH moiety, such that after a spatial barcode is added to the probe in situ, the constant region is located between the target identification and the extended spatial barcode. Without wishing to be bound by theory, the constant region can be used during sequence analysis to define the beginning of the spatial barcode domain.

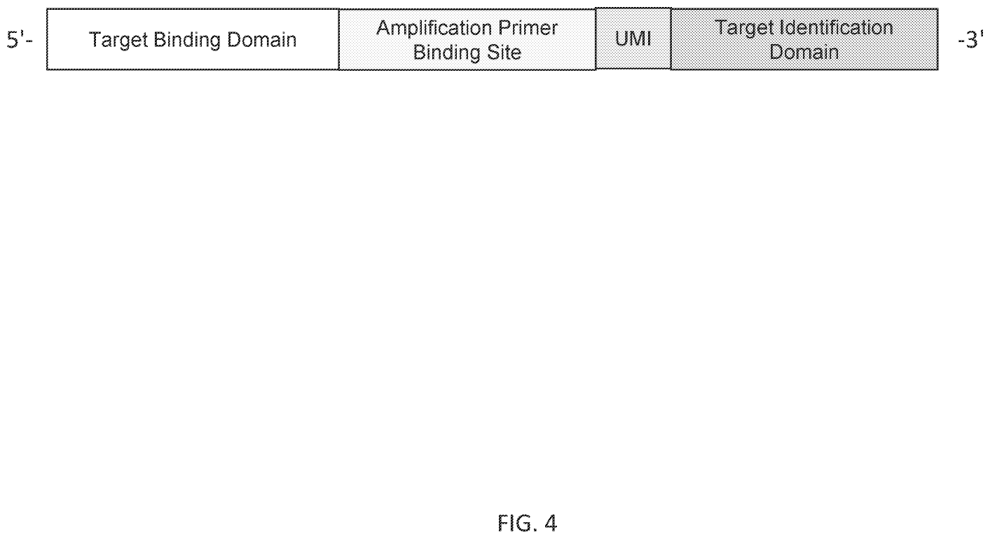

In some aspects of the methods of the present disclosure, a probe can be a single-stranded polynucleotide comprising, from 5′ to 3′, a target-binding domain, followed by an amplification primer binding site, followed by a unique molecular identifier, followed by a target identification domain. A schematic of the aforementioned probe is shown in FIG. 4.

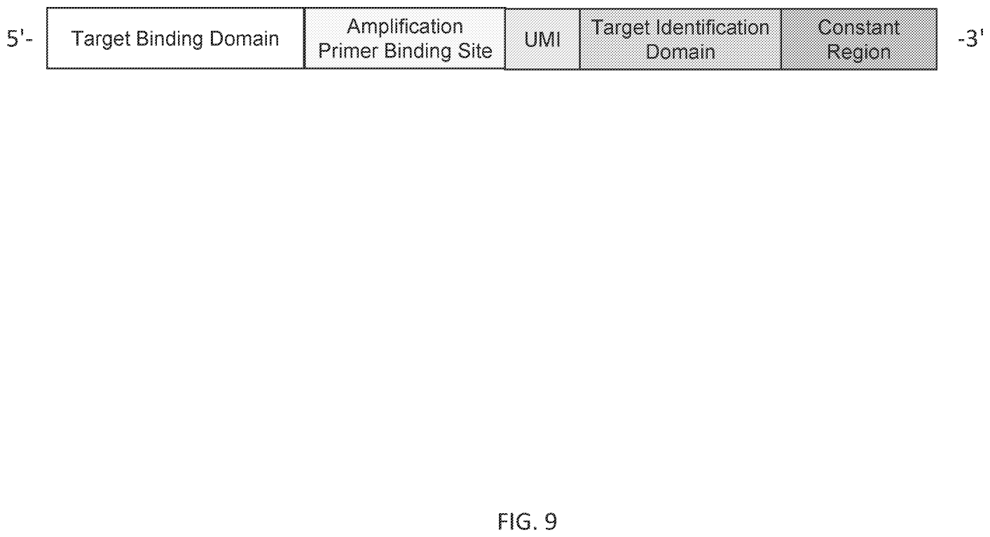

In some aspects of the methods of the present disclosure, a probe can be a single-stranded polynucleotide comprising, from 5′ to 3′, a target-binding domain, followed by an amplification primer binding site, followed by a unique molecular identifier, followed by a target identification domain, followed by a constant region. A schematic of the aforementioned probe is shown in FIG. 9.

In some aspects of the methods of the present disclosure a probe can be a single-stranded polynucleotide comprising, from 5′ to 3′, a target binding domain, followed by an amplification primer binding site, followed by a target identification domain, followed by a unique molecular identifier.

In aspects, probes are provided to a sample at concentrations typically less than that used for immunohistochemistry (IHC) or for in situ hybridization (ISH). Alternately, the concentration may be significantly less than that used for IHC or ISH. For example, the probe concentration may be 2-fold less, 5-fold less, 10-fold less, 20-fold less, 25-fold less, 30-fold less, 50-fold less, 60-fold less, 70-fold less, 80-fold less, 90-fold less, 100-fold less, 200-fold less, 300-fold less, 400-fold less, 500-fold less, 600-fold less, 700-fold less, 800-fold less, 900-fold less, 1000-fold less, 2000-fold less, or less and any number in between. In aspects, probes are provided at a concentration of 100 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 0.09 nM, 0.08 nM, 0.07 nM, 0.06 nM, 0.05 nM, 0.04 nM, 0.03 nM, 0.02 nM, 0.01 nM, and less and any concentration in between.

Background noise, during protein detection, can be reduced by performing a negative purification of the intact probe molecule. This can be done by conducting an affinity purification of the probe after collection of eluate from a region of interest.

In some aspects of the methods of the present disclosure at least one, or at least two, or at least three, or at least four, or at least five, or at least six, or at least seven, or at least eight, or at least nine, or at least ten, or at least eleven, or at least twelve, or at least thirteen, or at least fourteen, or at least fifteen, or at least sixteen, or at least seventeen, or at least eighteen, or at least nineteen, or at least twenty, or at least thirty, or at least forty, or at least fifty, or at least sixty, or at least seventy, or at least eighty, or at least ninety, or at least one hundred probes can bind to a single target analyte. As used herein, the term “tiling” is used to describe when more than one probe of the present disclosure is bound to a target analyte.

A set of probes, a plurality of probes, a solution comprising a plurality of probes can include at least one species of probes, i.e., directed to one target. A set of probes preferably includes at least two, e.g., 3, 4, 5, 6, 7, 8, 9, 10, 11, 12, 13, 14, 15, 16, 17, 18, 19 20, 30, 40, 50, 60, 70, 80, 90, 100, 200, 300, 400, 500, 600, 700, 800, 900, 1000 or more species of probes. A probe set may include one or multiple copies of each species of probe.

A first set of, a first plurality of or a first solution comprising probes only may be applied to a sample. Alternately, a second set (or higher number) of probes may be later applied to the sample. The first set and second (or higher number) may target only nucleic acids, only proteins, or a combination thereof.

In the present disclosure, two or more targets (i.e., proteins, nucleic acids, or a combination thereof) are detected; 3, 4, 5, 6, 7, 8, 9, 10, 11, 12, 13, 14, 15, 16, 17, 18, 19 20, 30, 40, 50, 60, 70, 80, 90, 100, 200, 300, 400, 500, 600, 700, 800, 900, 1000 or more targets, and any number there between, are detected.

A set of probes may be pre-defined based upon the cell type or tissue type to be targeted. For example, if the tissue is a breast cancer, then the set of probes will include probes directed to proteins relevant to breast cancer cells (e.g., Her2, EGFR, and PR) and/or probes directed to proteins relevant to normal breast tissues. Additionally, the set of probes may be pre-defined based upon developmental status of a cell or tissue to be targeted. Alternately, the set of probes may be pre-defined based upon subcellular localizations of interest, e.g., nucleus, cytoplasm, and membrane. For example, antibodies directed to Foxp3, Histone H3, or P-S6 label the nucleus, antibodies directed to CD3, CD4, PD-1, or CD45RO label the cytoplasm, and antibodies directed to PD-L1 label membranes.

### Spatial Barcode Domains

In some aspects of the methods of the present disclosure, a spatial barcode domain can comprise a spatial identifier sequence, wherein the spatial identifier sequence comprises a nucleic acid sequence that is unique to a specific location/region of interest within a tissue sample. In this way, when a probe on which a spatial barcode has been extended is collected and sequenced, the user can use the spatial identifier sequence of the spatial barcode domain to determine in what location/region of interest of the tissue sample the probe in when it was bound to its corresponding target analyte.

In some aspects, a spatial identifier sequence can comprise at least about 1, or at least about 2, or at least about 3, or at least about 4, or at least about 5, or at least about 6, or at least about 7, or at least about 8, or at least about 9, or at least about 10, or at least about 11, or at least about 12, or at least about 13, or at least about 14, or at least about 15, or at least about 16, or at least about 17, or at least about 18, or at least about 19, or at least about 20 nucleotides in length, or at least about 21, or at least about 22, or at least about 23, or at least about 24, or at least about 25, or at least about 26, or at least about 27, or at least about 28, or at least about 29, or at least about 30, or at least about 31, or at least about 32, or at least about 33, or at least about 34, or at least about 35, or at least about 36, or at least about 37, or at least about 38, or at least about 39 or at least about 40 nucleotides. In some aspects, a spatial identifier sequence can comprise at least about 20 nucleotides.

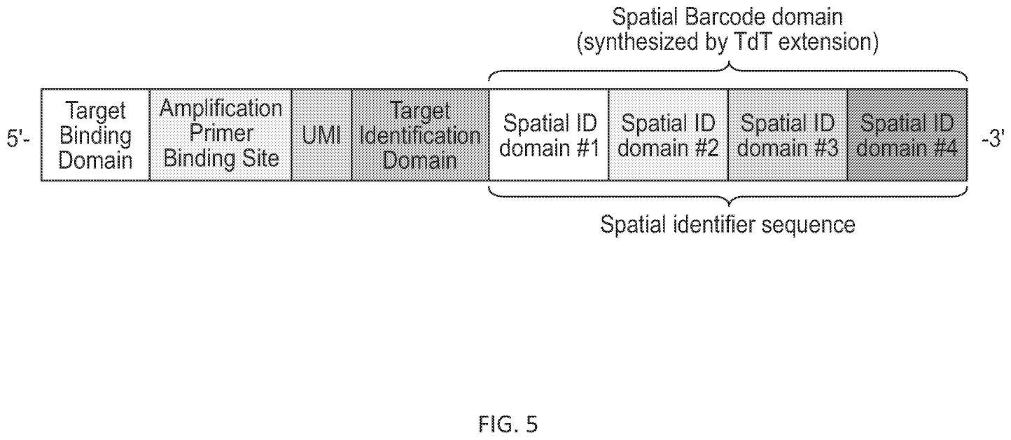

In some aspects of the methods of the present disclosure, a spatial identifier sequence can comprise at least one, at least two, at least three, at least four, at least five, at least six, at least seven, at least eight, at least nine, at least 10, at least 11, at least 12, at least 13, at least 14, at least 15, at least 16, at least 17, at least 18, at least 19 or at least 20 spatial identification domains. FIG. 5 is schematic of a probe of the present disclosure comprising a target binding domain, an amplification primer binding site, a unique molecular identifier and a target identification domain, along with a spatial barcode domain that has been synthesized by TdT extension in the methods of the present disclosure. In this non-limiting example, the spatial barcode domain comprises a spatial identifier sequence, wherein the spatial identifier sequence comprises four (#1, #2, #3 and #4) spatial identification domains.

A spatial identification domain can comprise at least about one, or at least about two, or at least about three, or at least about four, or at least about five, or at least about six, or at least about seven, or at least about eight, or at least about nine, or at least about ten, or at least about 11, or at least about 12, or at least about 13, or at least about 14, or at least about 15, or at least about 16 or at least about 17, or at least about 18, or at least about 19, or at least about 20 nucleotides. In some aspects, and spatial identification domain can comprise

In some aspects of the methods of the present disclosure, an individual spatial identification domain within a single spatial barcode domain can comprise the same number or a different number of nucleotides as compared to another individual spatial identification domain within that same single spatial barcode domain. In a non-limiting example, a spatial barcode domain may comprise four spatial identification domains, a first spatial identification domain, a second spatial identification domain, a third spatial identification domain and a fourth spatial identification domain. The first spatial identification domain may comprise two nucleotides, a second spatial identification domain may comprise three nucleotides, the third spatial identification domain may comprise two nucleotides and the fourth spatial identification domain may comprise four nucleotides. Thus, in this non-limiting example, the first spatial identification domain comprises the same number of nucleotides as the third spatial identification domain, but a different number of nucleotides as compared to the second spatial identification domain and the fourth spatial identification domain.

In some aspects of the methods of the present disclosure, each spatial identification domain within a single spatial barcode may comprise the same nucleotide at the 3′-terminus. In a non-limiting example, a spatial barcode domain may comprise four spatial identification domains, a first spatial identification domain, a second spatial identification domain, a third spatial identification domain, and fourth spatial identification domain. In this non-limiting example, each spatial identification domain comprises a “C” nucleotide at the 3′ terminus. Thus, the first spatial identification domain may comprise the nucleotide sequence 5′-ATC-3′, the second spatial identification domain may comprise the nucleotide sequence 5′-TGC-3′, the third spatial identification domain may comprise the nucleotide sequence S′-TAC-3′ and the fourth spatial identification domain may comprise the nucleotide sequence 5′-AAC-3′. Note in this non-limiting example each spatial identification domain in the spatial barcode comprises the same number of nucleotides.

In some aspects of the methods of the present disclosure, each spatial identification domain within any spatial barcode present within a sample may comprise the same nucleotide at the 3′-terminus.

In another non limiting example, a spatial barcode domain may comprise four spatial identification domains, a first spatial identification domain, a second spatial identification domain, a third spatial identification domain, and fourth spatial identification domain. In this non-limiting example, each spatial identification domain comprises a “C” nucleotide at the 3′ terminus. Thus, the first spatial identification domain may comprise the nucleotide sequence 5′-AC-3′, the second spatial identification domain may comprise the nucleotide sequence S′-GTC-3′, the third spatial identification domain may comprise the nucleotide sequence 5′-TATC-3′ and the fourth spatial identification domain may comprise the nucleotide sequence S′-AAC-3′. Note in this non-limiting example the spatial identification domains in the spatial barcode comprises differing number of nucleotides (the first spatial identification domain has two nucleotides, the second spatial identification domain has three nucleotides, the third spatial identification domain has four nucleotides, and the fourth spatial identification domain has three nucleotides) and each spatial identification domain has a “C” nucleotide at the 3′ terminus.

Without wishing to be bound by theory, whether each spatial identification domain within a spatial barcode has the same number of nucleotides or a differing number of nucleotides can determine the number of extension cycles that are needed and the number of different spatial barcodes that can be constructed, and consequently, the number of regions of interest that can be analyzed on a single tissue sample. For example, a spatial barcode domain may comprise 10 or 11 spatial identification domains, wherein each spatial identification domain comprises 1 nucleotide. This would allow for the creation of 410 to 411 unique spatial barcode sequences, meaning that 410 to 411 different regions of interest could be analyzed on a single tissue sample. In this configuration, 40-44 extensions cycles would be needed in order to construct all of the barcodes on the tissue sample. In another example, approximately 2.1 million unique spatial barcodes could be constructed if spatial identification domains could have differing numbers of nucleotides ranging from 1 nucleotide to 4 nucleotides. In this configuration, the spatial barcodes would be 7 to 28 nucleotides in total length, and only 28 extension cycles would be needed to construct all of the barcodes.

The spatial barcode domain corresponding a first location in a tissue sample may have more than, less than, or the same number of spatial identification domains as a spatial barcode domain corresponding to a second location in a tissue sample.

A spatial barcode domain can comprise a nucleic acid sequence that is specific and unique to a single location/region of interest in a tissue sample. For example, the combined nucleic acid sequence of all of the spatial identification domains within a spatial barcode domain can be specific and unique to a single location/region of interest in a tissue sample. In this way, when a probe on which a spatial barcode has been extended is collected and sequenced, the user can use the sequence of the spatial barcode domain to determine in what location/region of interest of the tissue sample the probe was located in when it was bound to its corresponding target analyte.

A spatial identification domain can comprise at least about one, or at least about two, or at least about three, or at least about four, or at least about five, or at least about six, or at least about seven, or at least about eight, or at least about nine, or at least about ten, or at least about 11, or at least about 12, or at least about 13, or at least about 14, or at least about 15, or at least about 16 or at least about 17, or at least about 18, or at least about 19, or at least about 20 nucleotides.

In some aspects of the methods of the present disclosure, the methods can further comprise, after the spatial barcode domains are synthesized in each location of the tissue sample (e.g. step (f)), repeating steps (b)-(e) to extend the spatial barcode domain in each location of the tissue sample such that the spatial barcode domain comprises, at the 3′ end, a delimiting domain.

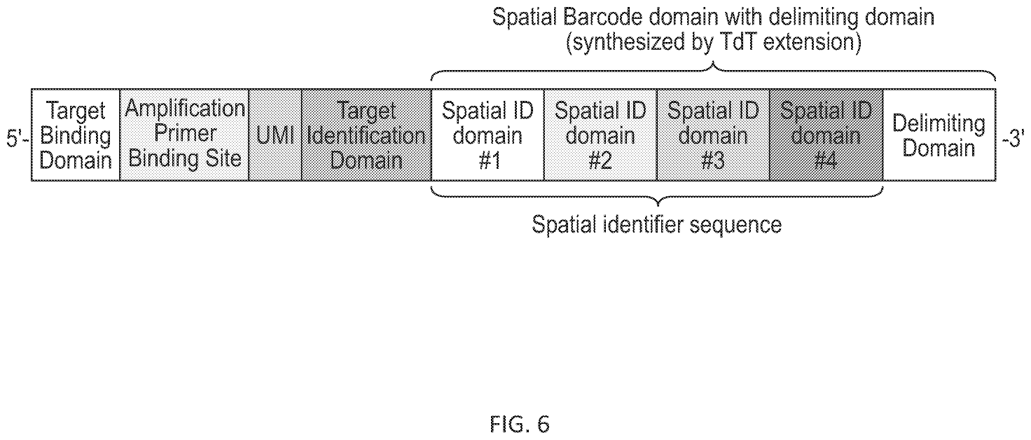

A delimiting domain can comprise a specific nucleic acid sequence that is the same for each location of the tissue sample. A delimiting domain can be at least 1, or at least 2, or at least 3, or at least 4, or at least 5, or at least 6, or at least 7, or at least 8, or at least 9, or at least 10, or at least 11 or at least 12, or at least 13, or at least 14, or at least 15, or at least 16, or at least 17, or at least 18, or at least 19, or at least 20 nucleotides in length. A delimiting domain can comprise any nucleic acid sequence. In a non-limiting example, the delimiting domain can comprise the nucleic acid sequence “TCTC”. FIG. 6 is a schematic of the probe of FIG. 5, wherein the spatial barcode domain has been further extended by TdT synthesis to include a delimiting domain.

Without wishing to be bound by theory, the sequence of a delimiting domain can be used during sequencing analysis to define the end of the spatial barcode domain, thereby aiding in the quantification via sequencing of the collected probes.

In some aspects of the methods of the present disclosure, the methods can further comprise, after the spatial barcode domains are synthesized in each location of the tissue sample (e.g. step (f)), extending the spatial barcode domain in each location of the tissue sample such that the spatial barcode domain comprises a polyT domain or other amplification primer binding site.

In some aspects of the methods of the present disclosure, the methods can further comprise after the spatial barcode domains are synthesized in each location of the tissue sample (e.g. step (f)): (i) repeating steps (b)-(e) to extend the spatial barcode domain in each location of the tissue sample such that the spatial barcode domain comprises, a delimiting domain; and (ii) extending the spatial barcode domain in each location of the tissue sample such that the spatial barcode domain comprises a polyT domain.

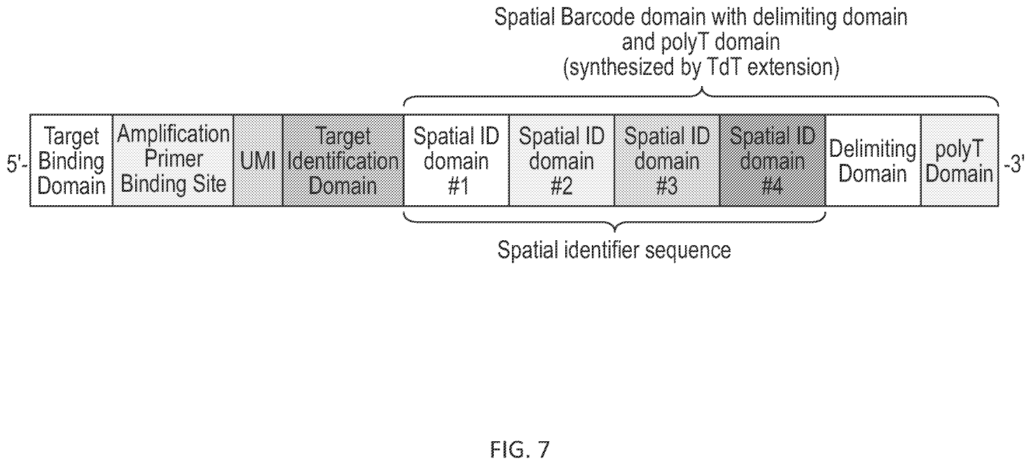

FIG. 7 is a schematic of the probe of FIG. 5, wherein the spatial barcode domain has been further extended by TdT synthesis to include a delimiting domain and then further extended by TdT synthesis to include a polyT domain.

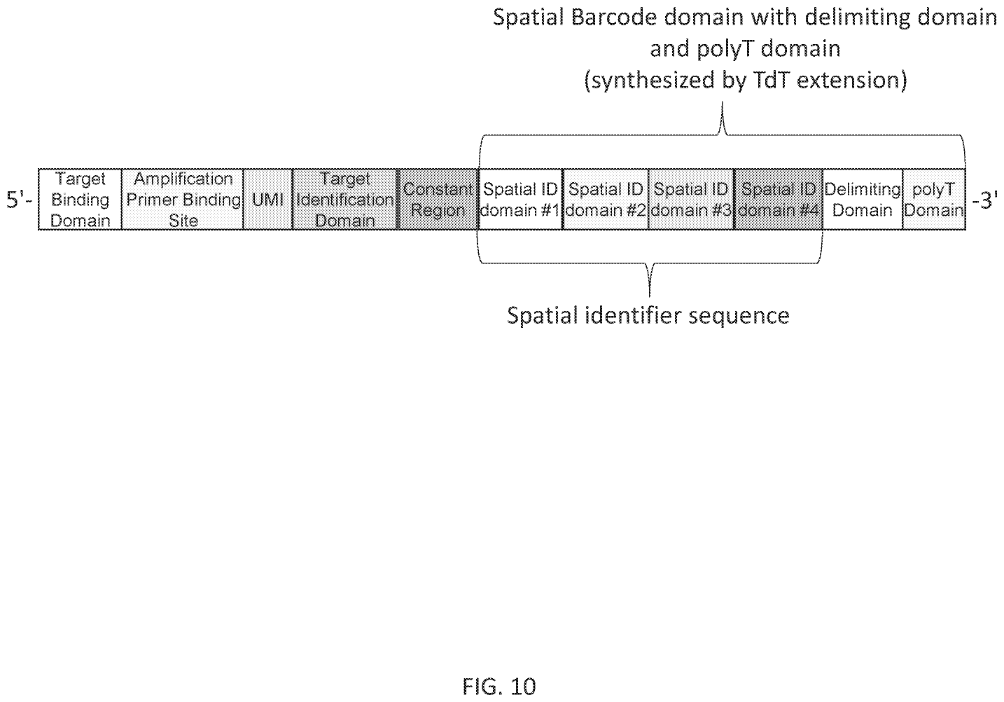

FIG. 10 is a schematic of the probe of FIG. 9, wherein the probe has been extended by TdT synthesis in the methods of the present disclosure to include a spatial barcode domain, wherein the spatial barcode domain comprises four spatial identification domains, a delimiting domain and a polyT domain.

In some aspects, a polyT domain is a polynucleotide that is comprised of only T nucleotides. In some aspects, the polyT domain can comprise at least about 1, or at least about 2, or at least about 3, or at least about 4, or at least about 5, or at least about 6, or at least about 7, or at least about 8, or at least about 9, or at least about 10, or at least about 11 or at least about 12, or at least about 13, or at least about 14, or at least about 15, or at least about 16, or at least about 17, or at least about 18, or at least about 19, or at least about 20 nucleotides, or at least about 25, or at least about 30, or at least about 35, or at least about 40, or at least about 50, or at least about 55, or at least about 60, or at least about 65, or at least about 70, or at least about 75, or at least about 80, or at least about 85, or at least about 90, or at least about 95, or at least about 100, or at least about 125, or at least about 150, or at least about 175, or at least about 200, or at least about 225, or at least about 250, or at least about 275, or at least about 300, or at least about 350, or at least about 400, or at least about 450, or at least about 500, or at least about 2000 T nucleotides. In some aspects, a polyT domain comprises more than 20 but less than 500 T nucleotides. In some aspects, a polyT domain comprises more than 20 but less than 2000 T nucleotides.

In some aspects, a polyT domain of a spatial barcode domain of a first bound probe can the same length or a different length than a polyT domain of a spatial barcode domain of a second bound probe.

In some aspects, extending the spatial barcode domain in each location of the tissue sample such that the spatial barcode domain comprises a polyT domain can comprise contacting the sample with a solution comprising dTTP and TdT.

### Amplification Methods

In some aspects of the methods of the present disclosure, quantifying via sequence can comprise amplifying the probes collected from the tissue sample.

In some aspects, amplifying the probes collected from the tissue sample can comprise amplifying the collected probes using a first amplification primer and a second amplification primer. In some aspects amplifying the probes collected from the tissue sample can comprise contacting the collected probes with a first amplification primer and a second amplification primer, wherein the first amplification primer and the second amplification primer hybridize to the collected probes. In some aspects, amplifying the probes collected from the tissue sample can comprise amplification reactions known in the art, including, but not limited to PCR.

In some aspects of the methods of the present disclosure, an amplification can comprise at least one NGS index sequence. In some aspects, the NGS index sequence is an is index sequence or an i7 index sequence. In some aspects, the NGS index sequence can be any index sequence known in the art.

In some aspects, an NGS index sequence can be at least about 1, or at least about 2, or at least about 3, or at least about 4, or at least about 5, or at least about 6, or at least about 7, or at least about 8, or at least about 9, or at least about 10, or at least about 11, or at least about 12, or at least about 13, or at least about 14, or at least about 15, or at least about 16, or at least about 17, or at least about 18, or at least about 19, or at least about 20 nucleotides in length. In some aspects, an NGS index sequence is at least about 8 nucleotides in length. In some aspects, an NGS index sequence is at least about 10 nucleotides in length.

In some aspects of the methods of the present disclosure, an amplification primer can comprise an i5 index sequence, wherein the i5 sequence comprises the sequence set forth in any one of SEQ ID NOs: 8-13 and 20-10,404, or the reverse complement thereof.

TABLE 1
Exemplary i5 index sequences

| | SEQ ID |
|---|---|
| Sequence | NO: |
| GAATGCACGA | 8 |
| AAGACTATAG | 9 |
| TCGGCAGCAA | 10 |
| CTAATGATGG | 11 |
| GGTTGCCTCT | 12 |
| CGCACATGGC | 13 |

In some aspects of the methods of the present disclosure, an amplification primer can comprise an i7 index sequence, wherein the i7 sequence comprises the sequence set forth in any one of SEQ ID NOs: 14-10,404, or the reverse complement thereof.

TABLE 2
Exemplary i7 index sequences

| | SEQ ID |
|---|---|
| Sequence | NO: |
| CGGCAATGGA | 14 |
| GCCGTAACCG | 15 |
| AACCATTCTC | 16 |
| GGTTGCCTCT | 17 |
| CTAATGATGG | 18 |
| TCGGCCTATC | 19 |

In some aspects of the methods of the present disclosure, an amplification primer can comprise a sequencing primer binding site. In some aspects, a sequencing primer binding site can be at least about 1, or at least about 2, or at least about 3, or at least about 4, or at least about 5, or at least about 6, or at least about 7, or at least about 8, or at least about 9, or at least about 10, or at least about 11, or at least about 12, or at least about 13, or at least about 14, or at least about 15, or at least about 16, or at least about 17, or at least about 18, or at least about 19, or at least about 20 nucleotides in length, or at least about 21, or at least about 22, or at least about 23, or at least about 24, or at least about 25, or at least about 26, or at least about 27, or at least about 28, or at least about 29, or at least about 30, or at least about 31, or at least about 32, or at least about 33, or at least about 34, or at least about 35, or at least about 36, or at least about 37, or at least about 38, or at least about 39 or at least about 40 nucleotides in length.

In some aspects, a sequencing primer binding site is at least about 9 nucleotides in length. In some aspects, a sequencing primer binding site is at least about 34 nucleotides in length.

In some aspects of the methods of the present disclosure, an amplification primer can comprise a flow cell adapter sequence, wherein the flow cell adapter sequence is suitable for sequencing. At least one amplification primer used in the methods of the present disclosure can comprise a P5 flow cell adapter sequence, wherein the PS flow cell adapter sequence comprises the sequence set forth in SEQ ID NO: 10,405, or the reverse complement thereof. At least one amplification primer used in the methods of the present disclosure can comprises a P7 flow cell adapter sequence, wherein the P7 flow cell adapter sequence comprises the sequence set forth in SEQ ID NO: 10,406, or the reverse complement thereof.

In some aspects, a flow cell adapter sequence can comprise between about 15 to about 40 nucleotides. A flow cell adapter sequence can comprise about 29 nucleotides. A flow cell adapter sequence can comprise about 24 nucleotides.

In some aspects of the methods and compositions of the present disclosure, a flow cell adapter sequence suitable for sequencing can comprise at least about 5, at least about 10, at least about 15, at least about 20, at least about 25, at least about 30, at least about 35 at least about 40, at least about 45, at least about 50, at least about 55, at least about 60, at least about 65, at least about 70, at least about 75, at least about 80, at least about 85, at least about 90, at least about 95 or at least about 100 nucleotides.

In some aspects, an amplification primer can comprise a poly A domain. In some aspects, a poly A domain is a polynucleotide that is comprised of only A nucleotides. In some aspects, the poly A domain can comprise at least about 1, or at least about 2, or at least about 3, or at least about 4, or at least about 5, or at least about 6, or at least about 7, or at least about 8, or at least about 9, or at least about 10, or at least about 11 or at least about 12, or at least about 13, or at least about 14, or at least about 15, or at least about 16, or at least about 17, or at least about 18, or at least about 19, or at least about 20 nucleotides, or at least about 25, or at least about 30, or at least about 35, or at least about 40 A nucleotides. In some aspects, a poly A domain can comprise at last about 32 nucleotides. In some aspects, a polyA domain can be used to hybridize a sequence probe to an extended spatial barcode domain by hybridizing the poly A domain to a polyT domain.

In some aspects, an amplification primer can comprise a degenerate base. In some aspects, an amplification primer can comprise at least about two degenerate bases. In some aspects, an amplification primer can comprise at least about three, or at least about four, or at least about five, or at least about six, or at least about seven, or at least about eight, or at least about nine, or at least about ten degenerate bases. In some aspects, the degenerate base(s) can be located at the 3′ end of an amplification primer. In some aspects, in the context of a plurality of amplification primers, a degenerate base position may have an A, a T, a C or a G nucleotide within the plurality. In some aspects, in the context of a plurality of amplification primers, a degenerate base position may have an A, a C or a G nucleotide.

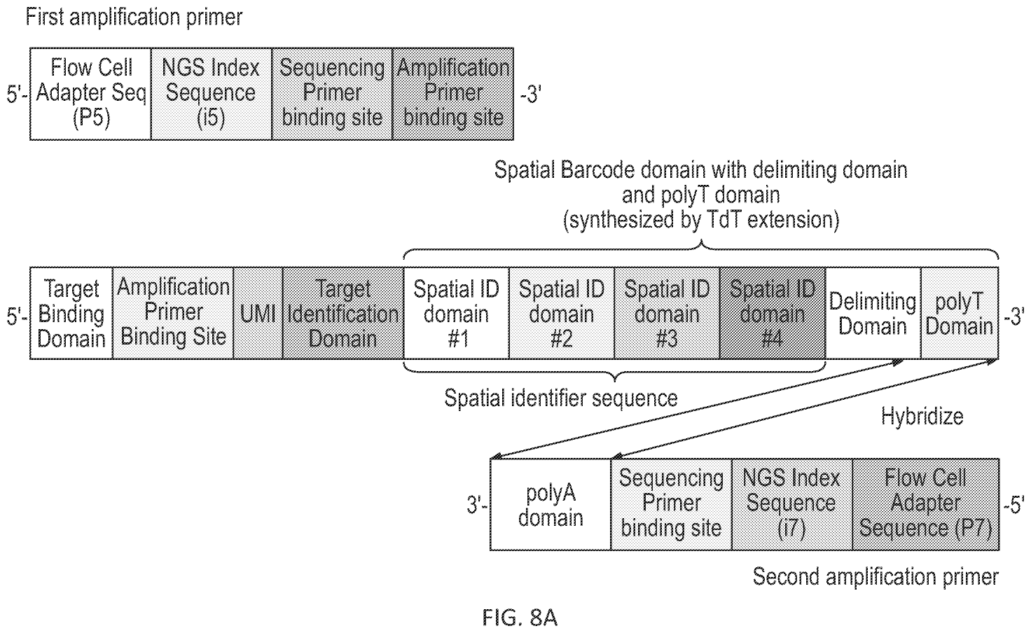

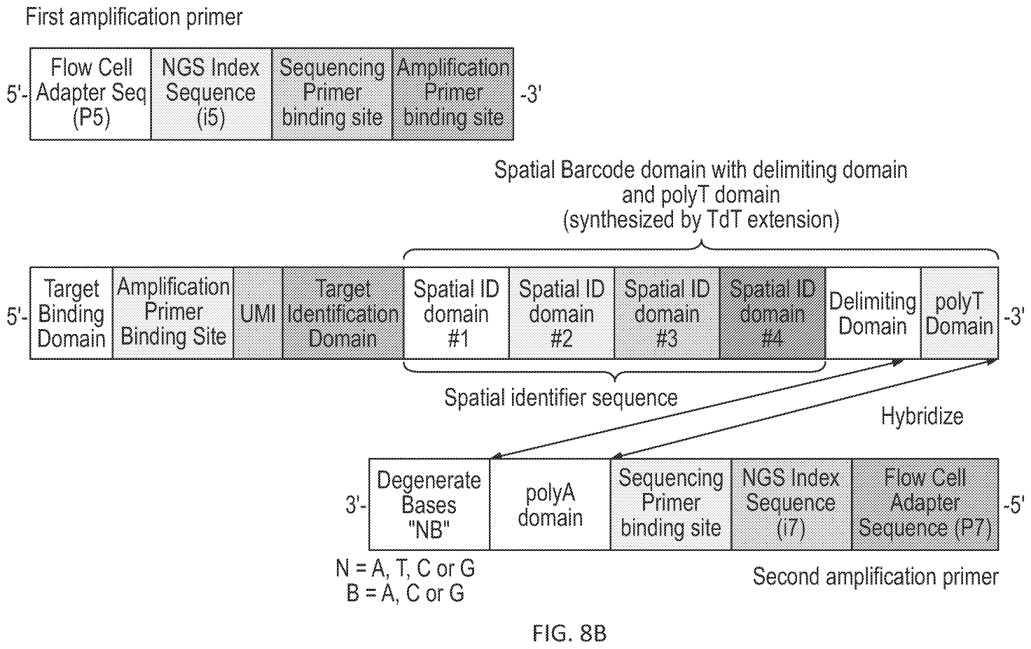

FIG. 8A is an exemplary schematic of a method of amplifying the probes collected from the tissue sample as part of quantifying the probes via sequencing. In this non-limiting example, the probe has a target binding domain, followed by an amplification primer binding site, followed by a unique molecular identifier, followed by a target identification domain. In this non-limiting example, a spatial barcode has been synthesized and extended on the 3′ end of the probe. The spatial barcode domain has been synthesized by TdT extension according to the methods of the present disclosure and comprises a spatial identifier sequence comprising four (#1, #2, #3 and #4) spatial identification domains, a delimiting domain and a polyT domain. In this non-limiting example, a first amplification primer has a flow cell adapter sequence (P5), followed by an NGS index sequence (15), followed by a sequencing primer binding site, followed by a sequence that matches the amplification primer bindings site on the probe. In this non-limiting example, a second amplification primer has a polyA domain, followed by a sequencing primer binding site, followed by an NGS index sequence (17), followed by a Flow Cell Adapter sequence (P7). The amplification primers can be hybridized to the probes as shown and used to amplify the probe using standard techniques, including, but not limited to, PCR, as would be appreciated by the skilled artisan.

FIG. 8B is an exemplary schematic of a method of amplifying the probes collected from the tissue sample as part of quantifying the probes via sequencing. In this non-limiting example, the probe has a target binding domain, followed by an amplification primer binding site, followed by a unique molecular identifier, followed by a target identification domain. In this non-limiting example, a spatial barcode has been synthesized and extended on the 3′ end of the probe. The spatial barcode domain has been synthesized by TdT extension according to the methods of the present disclosure and comprises a spatial identifier sequence comprising four (#1, #2, #3 and #4) spatial identification domains, a delimiting domain and a polyT domain. In this non-limiting example, a first amplification primer has a flow cell adapter sequence (P5), followed by an NGS index sequence (15), followed by a sequencing primer binding site, followed by a sequence that matches the amplification primer bindings site on the probe. In this non-limiting example, a second amplification primer has two degenerate bases (3′-NB-5′, wherein Nis A. T. C or G and B is A, C or G), followed by a polyA domain, followed by a sequencing primer binding site, followed by an NGS index sequence (17), followed by a Flow Cell Adapter sequence (P7). The degenerate bases are located at the 3′-terminus of the second amplification primer. The amplification primers can be hybridized to the probes as shown and used to amplify the probe using standard techniques, including, but not limited to, PCR, as would be appreciated by the skilled artisan.

In some aspects of the methods of the present disclosure, an amplification primer can comprise an affinity moiety. An affinity moiety can include, but is not limited to, biotin. In aspects wherein an amplification primer comprises an affinity moiety, the affinity moiety can be used to further purify the amplified probes using a reagent that specifically binds to the affinity moiety. In some aspects wherein the affinity moiety is biotin, streptavidin-coated beads can be used to purify the amplified probes.

### Illumination Methods

In some aspects of the methods of the present disclosure, illuminating a location of a tissue sample can comprise illuminating the location using a two-photon excitation method. In some aspects, the two-photon excitation method is a two-photon excitation method using light that has a near-infrared (NIR) wavelength. In some aspects, a NIR wavelength can be about 700 to about 1000 nm. Without wishing to be bound by theory, two-photo excitation allows for improved spatial confinement and minimized excitation crosstalk as compared with one-photon excitation methods, including one-photon excitation methods at UV (300-405 nm) wavelengths. Thus, spatial barcodes can be synthesized in smaller, more defined regions of the tissue sample, increasing the spatial resolution and accuracy of the methods of the present disclosure.

In some aspects, a two-photon excitation method can be a patterned two-photon illumination method. A patterned two-photon illumination method can comprise raster scanning a point of excitation light over a sample. This raster scanning can be achieved through the use of galvanometric mirrors, micro-electro-mechanical systems (MEMS) mirrors, acousto-optic deflectors or any combination thereof. A patterned two-photon illumination method can comprise rapidly modulating a laser on and off on a per-pixel basis. Such modulating can be achieved through the use of an acousto-optic modulator, a Pockels cell or any combination thereof. In some aspects, a patterned two-photon illumination method can comprise raster scanning a point of excitation light over a sample and rapidly modulating a laser on and off on a per-pixel basis.

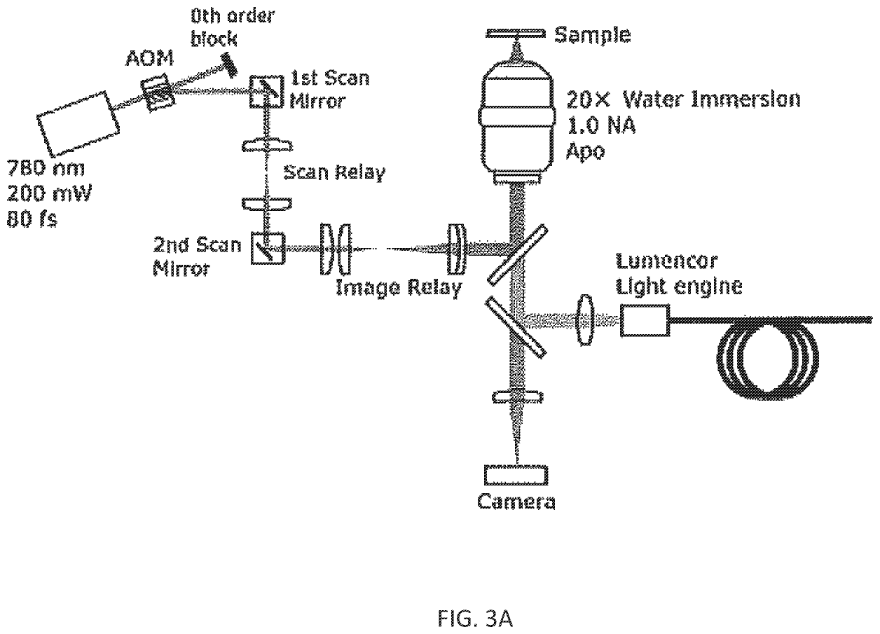

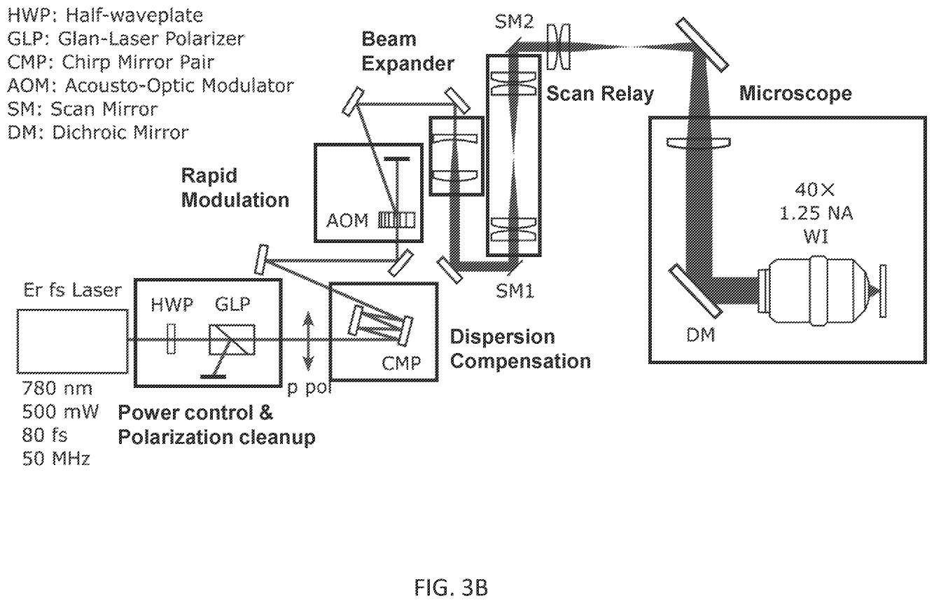

Exemplary optical schematics of a two-photon excitation method is shown in FIGS. 3A and 3B.

FIG. 3A is an exemplary optical schematic of a two-photon excitation method that can be used in the methods of the present disclosure. A femtosecond pulsed (<100 fs) erbium laser emitting at 780 nm is scanned across the sample plane in a raster pattern with a pair of 2D galvanometric mirrors (1st and 2nd Scan Mirror). Digital image projection for photocleaving is generated by modulating the laser intensity at each pixel using a rapid optical shutter (acousto-optic modulator, AOM) with <100 ns rise/fall time. The output of the scanned laser is sent into a commercial EPI fluorescent microscope base and projected onto the sample with a 20× 1.0 NA water immersion objective.

FIG. 3B is an exemplary optical schematic of a two-photon excitation method that can be used in the methods of the present disclosure. A femtosecond pulsed (<100 fs) erbium laser emitting at 780 nm is scanned across the sample plane in a raster pattern with a pair of 2D galvanometric mirrors (SM1 and SM2). Digital image projection for photocleaving is generated by modulating the laser intensity at each pixel using a rapid optical shutter (acousto-optic modulator, AOM) with <100 ns rise/fall time. Polarization optics (HWP & GLP) and a negative group dispersion delay mirror pair (CMP) are used to cleanup polarization and compress the pulse at sample plane, respectively. The output of the scanned laser is sent into a commercial EPI fluorescent microscope base (Nikon Ti2) and projected onto the sample with a 40× 1.25 NA water immersion objective. The maximum field of view is 370×370 μm2, while the diffraction limited resolution is 380 nm. Achievable minimum cleaving feature resolution, measured as transition width from 10% to 90%, is less than 700 nm.

In some aspects of the methods of the present disclosure, a two-photon excitation method can comprise the use of erbium fiber laser. In some aspects, an erbium fiber laser can emit light at a wavelength of about 1550 nm. In some aspects, the light from an erbium fiber laser can be frequency doubled to about 775 nm with pulse widths less than 100 femtoseconds.

In some aspects of the methods of the present disclosure, a two-photon excitation method can comprise the use of a 780 nm fiber-based femtosecond laser. In some aspects, the repetition rate of the laser can be specified from 10 to 80 MHz with clean pulse widths of less than 90 femtoseconds. In some aspects, the beam quality, M² factor, can be less than 1.2. In some aspects, the beam diameter can be about 1.57 mm. In some aspects, the beam circularity can be at least 94%.

The application of two photon illumination in the present invention resulted in three surprising improvements in the ability to detect and quantify protein and/or nucleic acid expression in a user-defined region of a tissue, user-defined cell, and/or user-defined subcellular structure within a cell. First, two photon illumination as used in the present invention provides dramatic improvements in Z and modest improvements in X-Y resolution. Second, with two photon illumination of the background in the whole field of view away from the features is also lower Third and perhaps most significant, with one-photon UV illumination, there are unexpected diffraction, scattering and/or light piping phenomena caused by cellular structures. These artifacts can significantly degrade the ability to focus UV light below diffraction expectations. These tissue artifacts are not present with two photon illumination as used in the present invention.

In some aspects, illuminating a location of a tissue sample can comprise illuminating the location using a light source selected from the group consisting of an arc-lamp, a laser, a focused UV light source, and light emitting diode. In some aspects, illuminating a location of a tissue sample can comprise illuminating the location using a light emitting diode (LED).

In some aspects, a location of a tissue sample can be illuminated with UV light of a power of at least about 1 W/cm², or at least about 10 W/cm², or at least about 20 W/cm², or at least about 30 W/cm², or at least about 40 W/cm², or at least about 50 W/cm², or at least about 60 W/cm², or at least about 70 W/cm², or at least about 80 W/cm², or at least about 90 W/cm², or at least about 100 W/cm². In some aspects, a location of a tissue sample can be illuminated with UV light of a power of about 1 W/cm², or about 10 W/cm², or about 20 W/cm², or about 30 W/cm², or about 40 W/cm², or about 50 W/cm², or about 60 W/cm², or about 70 W/cm², or about 80 W/cm², or about 90 W/cm², or about 100 W/cm².

In some aspects, a location of a tissue sample can be illuminated with a UV light of a power of about 40.9 W/cm². In some aspects, a location of a tissue sample can be illuminated with a power of about 20.45 W/cm².

In some aspects, a location of a tissue sample can be illuminated with UV light for a total of about at least 1 second, or at least about 2 seconds, or at least about 3 seconds, or at least about 4 seconds, or at least about 5 seconds, or at least about 6 seconds, or at least about 7 seconds, or at least about 8 seconds, or at least about 9 seconds or at least about 10 seconds. In some aspects, a location of a tissue sample can be illuminated with UV light for a total of about 1 second, or about 2 seconds, or about 3 seconds, or about 4 seconds, or about 5 seconds, or about 6 seconds, or about 7 seconds, or about 8 seconds, or about 9 seconds or about 10 seconds.

In some aspects, a location of a tissue sample can be illuminated with UV light for a total of about 5 seconds.

In some aspects, a location of a tissue sample can be illuminated with UV light such that the illumination is provided for a duration of about 0.1 seconds with intervals between of about 0.4 seconds.

In some aspects of the methods of the present disclosure, illuminating a location of a tissue sample can comprise illuminating the location using a UV light in combination with nonlinear TdT extension. Without wishing to be bound by theory, nonlinear TdT extension can occur due to competing release and depletion of salts. Without wishing to be bound by theory, nonlinear TdT extension can occur due to competing release and depletion of nucleotides. Without wishing to be bound by theory, nonlinear TdT extension occurs due to competing process of release and depletion of TdT enzyme. By using nonlinear TdT extension, the methods of the present disclosure can be used to synthesize spatial barcodes in smaller, more defined regions of the tissue sample, increasing the spatial resolution beyond the theorical optical resolution of the present disclosure.

### Locations or Regions of Interest

As used herein, the terms “location of a tissue sample” and a “region of interest” are used interchangeably to refer to a specific and defined area of the tissue sample within which the abundance of one or more target analytes will be quantified.

In some aspects of the methods of the present disclosure, a region of interest can be no less than about 10 nm in the x and/or y direction, or no less than about 100 nm in the x and/or y direction, or no less than about 200 nm in the x and/or y direction, or no less than about 300 nm in the x and/or y direction, or no less than about 400 nm in the x and/or y direction, or no less than about 500 nm in the x and/or y direction, or no less than about 600 nm in the x and/or y direction, or no less than about 700 nm in the x and/or y direction, or no less than about 800 nm in the x and/or y direction, or no less than about 900 nm in the x and/or y direction, or no less than 1 μm in the x and/or y direction, or no less than 10 μm in the x and/or y direction, or no less than about 100 μm in the x and/or y direction, or no less than about 200 μm in the x and/or y direction, or no less than about 300 μm in the x and/or y direction, or no less than about 400 μm in the x and/or y direction, or no less than about 500 μm in the x and/or y direction, or no less than about 600 μm in the x and/or y direction, or no less than about 700 μm in the x and/or y direction, or no less than about 800 μm in the x and/or y direction, or no less than about 900 μm in the x and/or y direction. In some aspects of the methods of the present disclosure, an illuminated location of a tissue sample can be no less than about 10 nm in the x and/or y direction, or no less than about 100 nm in the x and/or y direction, or no less than about 200 nm in the x and/or y direction, or no less than about 300 nm in the x and/or y direction, or no less than about 400 nm in the x and/or y direction, or no less than about 500 nm in the x and/or y direction, or no less than about 600 nm in the x and/or y direction, or no less than about 700 nm in the x and/or y direction, or no less than about 800 nm in the x and/or y direction, or no less than about 900 nm in the x and/or y direction, or no less than 1 μm in the x and/or y direction, or no less than 10 μm in the x and/or y direction, or no less than about 100 μm in the x and/or y direction, or no less than about 200 μm in the x and/or y direction, or no less than about 300 μm in the x and/or y direction, or no less than about 400 μm in the x and/or y direction, or no less than about 500 μm in the x and/or y direction, or no less than about 600 μm in the x and/or y direction, or no less than about 700 μm in the x and/or y direction, or no less than about 800 μm in the x and/or y direction, or no less than about 900 μm in the x and/or y direction.

In some aspects of the methods of the present disclosure, a region of interest can be no less than about 10 nm in the z direction, no less than about 500 nm in the z direction, or no less than about 1500 nm in the z direction. In some aspects of the methods of the present disclosure, an illuminated location of a tissue sample can be no more than about 1500 nm in the z direction.

A region of interest may be a tissue type present in a sample, a cell type, a cell, or a subcellular structure within a cell.

Together, a comparison of the identity and abundance of the target proteins and/or target nucleic acids present in a first region of interest (e.g., tissue type, a cell type (including normal and abnormal cells), and a subcellular structure within a cell) and the identity and abundance of the target proteins and/or target nucleic acids present in second region of interest or more regions of interest can be made using the methods of the present disclosure.

In some aspects, a region of interest may comprise a single cell. In some aspects, a region of interest may include a plurality of cells, such as, but not limited to, no more than two cells, no more than three cells, no more than four cells, no more than five cells, no more than six cells, no more than seven cells, no more than eight cells, no more than nine cells or no more than ten cells.

In some aspects, a region of interest may comprise a subcellular structure within a single cell.

In some aspects, an area of the tissue sample may be manually selected by a user and each cell within the area would be automatically identified as a region of interest and encoded. Cells can be identified by specific staining, and software used to locate the boundaries of the cell. Thus, the methods and systems described herein can encode each cell differently with minimal user interaction.

### Probe Collection

In some aspects of the methods of the present disclosure, the step of collecting the probes bound to target analytes in the tissue sample can comprise scraping the tissue sample, digesting the tissue sample with proteinase K or a combination thereof. In some aspects, a proteinase K digestion can be performed at about 56° ° C. for at least about one hour, followed by a further digestion at about 90° ° C. for at least about 15 minutes.

In some aspects of the methods of the present disclosure, the step of collecting the probes bound to target analytes in the tissue sample can comprise an Ampure SPRI purification, as would be appreciated by the skilled artisan.

In some aspects of the methods of the present disclosure, the step of collecting the probes bound to target analytes in the tissue sample can comprise an xGen hybridization capture-based purification, as would be appreciated by the skilled artisan.

In some aspects, an xGen hybridization capture-based purification comprises hybridizing at least one capture probe to the probes bound to target analytes in the tissue sample.

In some aspects, a capture probe can comprise and affinity moiety. An affinity moiety can be biotin.

In some aspects, a capture probe can comprise at least one copy of a nucleic acid sequence that is complementary to a probe of the present disclosure. In some aspects, a capture probe can comprise at least about two copies of a nucleic acid sequence that is complementary to a probe of the present disclosure. In some aspects, a capture probe can comprise at least about three, or at least about four, or at least about five, or at least about six, or at least about seven, or at least about eight, or at least about nine, or at least about ten copies of a nucleic acid sequence that is complementary to a probe of the present disclosure. The nucleic acid sequence that is complementary to a probe of the present disclosure can be complementary to an amplification primer binding site located on the probe.

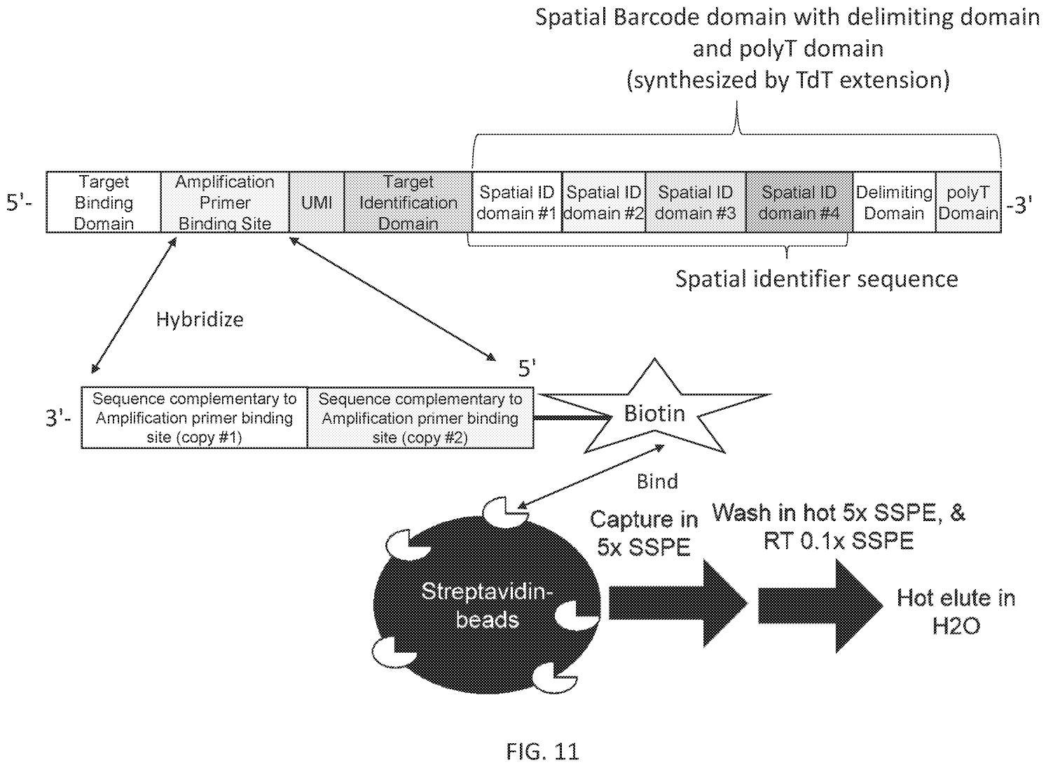

FIG. 11 is an exemplary schematic of a method of purifying the probes collected from the tissue sample as part of quantifying the probes via sequencing. In this non-limiting example, the probe has a target binding domain, followed by an amplification primer binding site, followed by a unique molecular identifier, followed by a target identification domain. In this non-limiting example, a spatial barcode has been synthesized and extended on the 3′ end of the probe. The spatial barcode domain has been synthesized by TdT extension and comprises a spatial identifier sequence comprising four (#1, #2, #3 and #4) spatial identification domains, a delimiting domain and a polyT domain. In this non-limiting example, a capture probe is used, wherein the capture probe comprises two copies of a sequence that is complementary to the amplification primer binding site on the probe. The capture probe further comprises a biotin moiety. The capture probe is hybridized to the probe, and then the biotin moiety is bound to streptavidin-beads in 5×SSPE buffer. The beads can then be washed in hot 5×SSPE and room temperature (RT) 0.1×SSPE. The probes can then be eluted from the streptavidin beads in hot H₂O.

### Tissue Sample Pre-Treatment

In some aspects of the methods of the present disclosure, the step of contacting a tissue sample with at least one probe or a plurality of probes can comprise incubating the tissue sample with the at least one probe or the plurality of probes for at least about one hour, or at least about two hours, or at least about three hours, or at least about four hours, or at least about five hours, or at least about six hours, or at least about seven hours, or at least about eight hours, or at least about nine hours, or at least about 10 hours, or at least about 11 hours, or at least about 12 hours, or at least about 24 hours, or at least about 36 hours, or at least about 48 hours.

In some aspects of the methods of the present disclosure, the step of contacting a tissue sample with at least one probe or a plurality of probes can comprise contacting the tissue sample with the at least one probe or a plurality of probes in combination with blocking DNA that has been terminated with ddTTP.

In some aspects of the methods of the present disclosure, prior to contacting a tissue sample with at least one probe or a plurality of probes, the tissue sample can be subjected to ddTTP (dideoxthymidine-triphosphate) termination. ddTTP termination can comprise contacting the tissue sample with ddTTP and TdT. Without wishing to be bound by theory, ddTTP termination is performed to remove block free 3′-OH moieties of endogenous nucleic acids within the tissue sample, preventing the endogenous nucleic acids from being extended during the extension of the spatial barcodes.

In some aspects of the methods of the present disclosure, the step of collecting the probes bound to target analytes in the tissue sample can comprise ExoIII digestion. Without wishing to be bound by theory, ExoIII digestion allows for the removal of dsDNA and the enrichment of ssDNA or dsDNA with a protruding 3′ overhang.

### Landmarking Probes and Methods

In some aspects, the methods of the present disclosure can further comprise incubating a sample with at least one general landmarking probe of the present disclosure.

In some aspects, the methods of the present disclosure can further comprise detecting at least one general landmarking probe bound within a sample.

In some aspects, the detection of at least one general landmarking probe bound within the sample can be used to define the boundaries of individual cells within a sample. By detecting the boundaries of individual cells within a sample, spatial patterns can be determined for downstream synthesis of spatial barcodes within the sample. In some aspects, the detection of one or more general landmarking probes in a sample can be used to generate a fluorescent cell image that subsequently collected gene expression data can then be mapped onto to create a spatially-resolved profile of gene expression.

In some aspects, a general landmarking probe can comprise a detectable label.

In some aspects, the detectable label can be a fluorescent label. Examples of fluorescent labels include, but are not limited to, fluorescent proteins, fluorescent nanoparticles, fluorophores, and any other fluorescent moiety known in the art.

In some aspects, the detectable label can be a calorimetric label. In some aspects, the detectable label can be a chemiluminescent label. In some aspects, the detectable label can be a radioactive label.

In some aspects, a general landmarking probe can comprise at least one antibody. The antibody can specifically bind to at least one cellular marker. In some aspects, the at least one antibody can be linked, directly or indirectly, to at least one detectable label.

A cellular marker can be a cytoplasmic marker. A cellular marker can be any cytoplasmic marker known in the art. Examples of cytoplasmic markers include, but are not limited to, specific proteins that are located exclusively or primarily within the cytoplasm of a cell. Specific proteins that are located exclusively or primarily within the cytoplasm of a cell can include, but are not limited to, microtubule proteins, vimentin, desmin and cytokeratin.

A cellular marker can be a membrane marker. A cellular marker can be any membrane marker known in the art. Examples of membrane markers include, but are not limited to, specific proteins that are exclusively or primarily on, associated with, or embedded in a specific membrane within a cell. Examples of such proteins include, but are not limited to, sodium-potassium ATPase, plasma membrane calcium ATPase (PMCA), proteins of the cadherin family, CD98, proteins associated with caveolae (e.g. caveolin), any integral membrane protein known in the art and any membrane-associated protein known in the art.

A cellular marker can be an organelle marker. An organelle marker can be any organelle marker known in the art. Examples of organelle markers include, but are not limited to, specific proteins that are exclusively or primarily associated with a specific organelle (e.g. Endoplasmic reticulum, golgi apparatus, mitochondria, ribosome, lysosome, endosomes, peroxisome, autophagosome, or any other organelle known in the art). Examples of such proteins include, but are not limited to, calreticulin (endoplasmic reticulum), GM130 (golgi apparatus), ATP5A (mitochondria), TOMM20 (mitochondria), RPS3 (ribosome), M6PR (lysosome), EEA1 (endosome), RAB7 (endosome), catalase (peroxisome), SQSTM1/p62 (autophagosome) and LC3B (autophagosome).

A cellular marker can be a nuclear marker. A nuclear marker can be any nuclear marker known in the art. Examples of nuclear markers include, but are not limited to, specific proteins that are exclusively or primarily associated with the nucleus of a cell, or a specific sub-structure within the nucleus of the cell. Examples of such proteins include, but are not limited to KDM1, NUP98, Lamin A, Lamin C, Lamin, SC35, Fibrillarin, HP1 alpha, CENPA or any other nuclear protein known in the art. Nuclear markers can also include, but are not limited to, nucleic acid stains such as DAPI or SYTO9 to stain cellular nucleic acids.

In some aspects, a general landmarking probe of the present disclosure can comprise a dye that targets one or more specific membrane(s) in a cell such as the plasma membrane, the nuclear membrane, the endoplasmic reticulum membrane, the lysosome membrane or any other membrane known in the art.

In some aspects, a general landmarking probe of the present disclosure can be detected in order to define the boundaries of individual cells within a sample. By detecting the boundaries of individual cells within a sample, spatial patterns can be determined for downstream synthesis of spatial barcodes within the sample. In some aspects, the detection of one or more general landmarking probes in a sample can be used to generate a fluorescent cell image that subsequently collected gene expression can then be mapped onto to create a spatially-resolved profile of gene expression.

### Samples

In some aspects of the present disclosure, samples may comprise any number of things, including, but not limited to cells (including both primary cells and cultured cell lines) and tissues (including cultured or explanted). In aspects, a tissue sample (fixed or unfixed) is embedded, serially sectioned, and immobilized onto a microscope slide. As is well known, a pair of serial sections will include at least one cell that is present in both serial sections. Structures and cell types, located on a first serial section will have a similar location on an adjacent serial section. The sample can be cultured cells or dissociated cells (fixed or unfixed) that have been immobilized onto a slide. A sample can be a formalin-fixed paraffin-embedded (FFPE) tissue sample.

In aspects, a tissue sample is a biopsied tumor or a portion thereof, i.e., a clinically-relevant tissue sample. For example, the tumor may be from a breast cancer. The sample may be an excised lymph node.

The sample can be obtained from virtually any organism including multicellular organisms, e.g., of the plant, fungus, and animal kingdoms; preferably, the sample is obtained from an animal, e.g., a mammal. Human samples are particularly preferred.

In some aspects, the probes, compositions, methods, and kits described herein are used in the diagnosis of a condition. As used herein the term diagnose or diagnosis of a condition includes predicting or diagnosing the condition, determining predisposition to the condition, monitoring treatment of the condition, diagnosing a therapeutic response of the disease, and prognosis of the condition, condition progression, and response to particular treatment of the condition. For example, a tissue sample can be assayed according to any of the probes, methods, or kits described herein to determine the presence and/or quantity of markers of a disease or malignant cell type in the sample (relative to the non-diseased condition), thereby diagnosing or staging a disease or a cancer.

In general, samples attached to a slide can be first imaged using fluorescence (e.g., fluorescent antibodies or fluorescent stains (e.g., DAPI)) to identify morphology, regions of interest, cell types of interest, and single cells and then expression of proteins and/or nucleic acids can be digitally counted from the sample on the same slide.

### Sequencing

In some aspects of the methods of the present disclosure, quantifying via sequencing can comprise using any known sequencing method in the art to determine the number of probes that were collected that a) correspond to a specific target analyte (i.e. comprise a specific target identification domain) and that b) correspond bound to a specific location/region of interest (i.e. comprise the same identifier domains within a spatial barcode domain.

Sequencing can be performed by any known sequencing method, including, but not limited to next-generation sequencing methods, sequencing by synthesis, massively parallel sequencing, or any other sequencing method known and practice by the skilled artisan.

In a preferred aspect, nucleic acid amplification can be solid-phase nucleic acid amplification. Thus, in further aspects the invention provides a method of solid-phase nucleic acid amplification of template polynucleotide molecules which comprises: preparing a library of template polynucleotide molecules which have common sequences at their 5′ and 3′ ends using the methods of the present disclosure and carrying out a solid-phase nucleic acid amplification reaction wherein said template polynucleotide molecules are amplified. Compositions and methods for nucleic acid amplification and sequencing have been described in, e.g., U.S. Pat. No. 9,376,678, which is incorporated herein by reference in its entirety.

The term “solid-phase amplification” as used herein refers to any nucleic acid amplification reaction carried out on or in association with a solid support such that all or a portion of the amplified products are immobilized on the solid support as they are formed. In particular, the term encompasses solid-phase polymerase chain reaction (solid-phase PCR), which is a reaction analogous to standard solution phase PCR, except that one or both of the forward and reverse amplification primers is/are immobilized on the solid support.

Although the invention encompasses “solid-phase” amplification methods in which only one amplification primer is immobilized (the other primer usually being present in free solution), it is preferred for the solid support to be provided with both the forward and the reverse primers immobilized. In practice, there will be a “plurality” of identical forward primers and/or a “plurality” of identical reverse primers immobilized on the solid support, since the PCR process requires an excess of primers to sustain amplification. References herein to forward and reverse primers are to be interpreted accordingly as encompassing a “plurality” of such primers unless the context indicates otherwise.

As will be appreciated by the skilled reader, any given PCR reaction requires at least one type of forward primer and at least one type of reverse primer specific for the template to be amplified. However, in certain aspects the forward and reverse primers may comprise template-specific portions of identical sequence and may have entirely identical nucleotide sequence and structure (including any non-nucleotide modifications). In other words, it is possible to carry out solid-phase amplification using only one type of primer, and such single-primer methods are encompassed within the scope of the invention. Other aspects may use forward and reverse primers which contain identical template-specific sequences, but which differ in some other structural features. For example, one type of primer may contain a non-nucleotide modification which is not present in the other.

In other aspects of the invention the forward and reverse primers may contain template-specific portions of different sequence.

Amplification primers for solid-phase PCR are preferably immobilized by covalent attachment to the solid support at or near the 5′ end of the primer, leaving the template-specific portion of the primer free for annealing to its cognate template and the 3′ hydroxyl group free for primer extension. Any suitable covalent attachment means known in the art may be used for this purpose. The chosen attachment chemistry will depend on the nature of the solid support, and any derivatization or functionalization applied to it. The primer itself may include a moiety, which may be a non-nucleotide chemical modification, to facilitate attachment. In one particularly preferred aspect the primer may include a sulphur-containing nucleophile, such as phosphorothioate or thiophosphate, at the S′ end. In the case of solid-supported polyacrylamide hydrogels (as described below), this nucleophile will bind to a “C” group present in the hydrogel. The most preferred means of attaching primers and templates to a solid support is via 5′ phosphorothioate attachment to a hydrogel comprised of polymerized acrylamide and N-(5-bromoacetamidylpentyl)acrylamide (BRAPA).

The terms “cluster” and “colony” are used interchangeably herein to refer to a discrete site on a solid support comprised of a plurality of identical immobilized nucleic acid strands and a plurality of identical immobilized complementary nucleic acid strands. The term “clustered array” refers to an array formed from such clusters or colonies. In this context the term “array” is not to be understood as requiring an ordered arrangement of clusters.

The invention also encompasses methods of sequencing the amplified nucleic acids generated by solid-phase amplification. Thus, the invention provides a method of nucleic acid sequencing comprising amplifying a library of nucleic acid templates by the methods of the present disclosure described above, using solid-phase amplification as described above to amplify this library on a solid support, and carrying out a nucleic acid sequencing reaction to determine the sequence of the whole or a part of at least one amplified nucleic acid strand produced in the solid-phase amplification reaction.

Sequencing, as referred to herein, can be carried out using any suitable “sequencing-by-synthesis” technique, wherein nucleotides are added successively to a free 3′ hydroxyl group, resulting in synthesis of a polynucleotide chain in the S′ to 3′ direction. The nature of the nucleotide added is preferably determined after each nucleotide addition.

The initiation point for the sequencing reaction may be provided by annealing of a sequencing primer to a product of the whole genome or solid-phase amplification reaction. In this connection, one or both of the adapters added during formation of the template library may include a nucleotide sequence which permits annealing of a sequencing primer to amplified products derived by whole genome or solid-phase amplification of the template library.

The products of solid-phase amplification reactions wherein both forward and reverse amplification primers are covalently immobilized on the solid surface are so-called “bridged” structures formed by annealing of pairs of immobilized polynucleotide strands and immobilized complementary strands, both strands being attached to the solid support at the S′ end. Arrays comprised of such bridged structures provide inefficient templates for nucleic acid sequencing, since hybridization of a conventional sequencing primer to one of the immobilized strands is not favored compared to annealing of this strand to its immobilized complementary strand under standard conditions for hybridization.

In order to provide more suitable templates for nucleic acid sequencing it is preferred to remove substantially all or at least a portion of one of the immobilized strands in the “bridged” structure in order to generate a template which is at least partially single-stranded. The portion of the template which is single-stranded will thus be available for hybridization to a sequencing primer. The process of removing all or a portion of one immobilized strand in a “bridged” double-stranded nucleic acid structure may be referred to herein as “linearization”.

Bridged template structures may be linearized by cleavage of one or both strands with a restriction endonuclease or by cleavage of one strand with a nicking endonuclease. Other methods of cleavage can be used as an alternative to restriction enzymes or nicking enzymes, including inter alia chemical cleavage (e.g. cleavage of a diol linkage with periodate), cleavage of abasic sites by cleavage with endonuclease, or by exposure to heat or alkali, cleavage of ribonucleotides incorporated into amplification products otherwise comprised of deoxyribonucleotides, photochemical cleavage or cleavage of a peptide linker.

It will be appreciated that a linearization step may not be essential if the solid-phase amplification reaction is performed with only one primer covalently immobilized and the other in free solution.

In order to generate a linearized template suitable for sequencing it is necessary to remove “unequal” amounts of the complementary strands in the bridged structure formed by amplification so as to leave behind a linearized template for sequencing which is fully or partially single stranded. Most preferably one strand of the bridged structure is substantially or completely removed.

Following the cleavage step, regardless of the method used for cleavage, the product of the cleavage reaction may be subjected to denaturing conditions in order to remove the portion(s) of the cleaved strand(s) that are not attached to the solid support. Suitable denaturing conditions will be apparent to the skilled reader with reference to standard molecular biology protocols.

Denaturation (and subsequent re-annealing of the cleaved strands) results in the production of a sequencing template which is partially or substantially single-stranded. A sequencing reaction may then be initiated by hybridization of a sequencing primer to the single-stranded portion of the template.

Thus, the nucleic acid sequencing reaction may comprise hybridizing a sequencing primer to a single-stranded region of a linearized amplification product, sequentially incorporating one or more nucleotides into a polynucleotide strand complementary to the region of amplified template strand to be sequenced, identifying the base present in one or more of the incorporated nucleotide(s) and thereby determining the sequence of a region of the template strand.

One preferred sequencing method which can be used in accordance with the invention relies on the use of modified nucleotides that can act as chain terminators. Once the modified nucleotide has been incorporated into the growing polynucleotide chain complementary to the region of the template being sequenced there is no free 3′-OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached a different label, known to correspond to the particular base, to facilitate discrimination between the bases added at each incorporation step. Alternatively, a separate reaction may be carried out containing each of the modified nucleotides separately.

The modified nucleotides may carry a label to facilitate their detection. Preferably this is a fluorescent label. Each nucleotide type may carry a different fluorescent label. However, the detectable label need not be a fluorescent label. Any label can be used which allows the detection of an incorporated nucleotide.

One method for detecting fluorescently labelled nucleotides comprises using laser light of a wavelength specific for the labelled nucleotides, or the use of other suitable sources of illumination. The fluorescence from the label on the nucleotide may be detected by a CCD camera or other suitable detection means.

The invention is not intended to be limited to use of the sequencing method outlined above, as essentially any sequencing methodology which relies on successive incorporation of nucleotides into a polynucleotide chain can be used. Suitable alternative techniques include, for example, Pyrosequencing, FISSEQ (fluorescent in situ sequencing), MPSS (massively parallel signature sequencing) and sequencing by ligation-based methods.

### Hybridization

As used herein, the term “hybridize” is used in its broadest sense to mean the formation of a stable nucleic acid duplex. In one aspect, “stable duplex” means that a duplex structure is not destroyed by a stringent wash under conditions such as, for example, a temperature of either about 5° ° C. below or about 5° C. above the Tm of a strand of the duplex and low monovalent salt concentration, e.g., less than 0.2 M, or less than 0.1 M or salt concentrations known to those of skill in the art A duplex can be “perfectly matched”, such that the polynucleotide and/or oligonucleotide strands making up the duplex form a double stranded structure with one another such that every nucleotide in each strand undergoes Watson-Crick base pairing with a nucleotide in the other strand. The term “duplex” comprises, but is not limited to, the pairing of nucleoside analogs, such as deoxyinosine, nucleosides with 2-aminopurine bases, PNAs, and the like, that can be employed. A duplex can comprise at least one mismatch, wherein the term “mismatch” means that a pair of nucleotides in the duplex fail to undergo Watson-Crick bonding.

As used herein, the term “hybridization conditions,” will typically include salt concentrations of less than about 1 M, more usually less than about 500 mM and even more usually less than about 200 mM. Hybridization temperatures can be as low as 5° C., but are typically greater than 22° ° C., more typically greater than about 30° C., and often in excess of about 37° C. Hybridizations are usually performed under stringent conditions, e.g., conditions under which a probe will specifically hybridize to its target analyte. Stringent conditions are sequence-dependent and are different in different circumstances. Longer fragments can require higher hybridization temperatures for specific hybridization. As other factors can affect the stringency of hybridization, including base composition and length of the complementary strands, presence of organic solvents and extent of base mismatching, the combination of parameters is more important than the absolute measure of any one alone. Certain hybridization conditions will promote the formation of a duplex between the entire length of a target binding domain and the target analyte. Other hybridization conditions will promote the formation of a duplex only along certain portions of the target binding domain.

### Compositions of the Present Disclosure

The present disclosure provides a composition comprising any of the probes, extended probes or probe-intermediates described herein.

The present disclosure provides a composition comprising a plurality of the probes, extended probes or probe-intermediates described herein.

### EXEMPLARY EMBODIMENTS

Embodiment 1. A method for in situ synthesis of a nucleic acid sequence in a tissue sample, the method comprising:
a) contacting the tissue sample with at least one probe,
   wherein the probe comprises a target-binding domain and a target identification domain,
   wherein the probe comprises a free 3′-OH moiety, and
   wherein the target-binding domain binds to at least one target molecule located at a first location of the tissue sample;
b) contacting the tissue sample with at least one reversible terminator nucleotide, at least one polymerase, at least one caged chelator-cofactor complex, and at least one unbound caged chelator,
   wherein the at least one caged chelator-cofactor complex comprises at least one cofactor bound to a caged chelator;
   wherein the at least one reversible terminator nucleotide comprises the nucleotide operably linked to a cleavable 3′ terminator moiety;
c) illuminating the first location of the tissue sample with light sufficient to uncage the at least one caged chelator-cofactor complex, thereby releasing the at least one cofactor,
   thereby activating the at least one polymerase,
   thereby ligating the at least one reversible terminator nucleotide to the free 3′-OH moiety of the at least one bound probe;
d) washing the tissue sample;
e) treating the tissue sample under conditions sufficient to cleave the 3′ terminator moiety of the at least one reversible terminator nucleotide ligated in step (c), thereby exposing a free 3′-OH moiety on the extended at least one bound probe; and
f) repeating steps (b)-(e) until the nucleic acid sequence has been synthesized.

Embodiment 2. The method of embodiment 1, wherein the target-binding domain binds to at least one target molecule at an at least second location of the tissue sample, and wherein the method further comprises repeating steps (b)-(f) at the at least second location.

Embodiment 3. The method of any one of the preceding embodiments, wherein the nucleic acid sequence synthesized at the first location of the tissue sample is different than the nucleic acid sequence synthesized at the at least second location of the tissue sample.

Embodiment 4. A method of producing a spatially-resolved profile of the abundance of at least two target analytes in a first and an at least second location of a tissue sample comprising:
a) contacting the tissue sample with a solution comprising at least two species of probes, the probes comprising a target-binding domain and a target identification domain,
   wherein each species of probe comprises a unique target-binding domain that binds to one of the at least two target analytes and a unique target identification domain specific for the target analyte, and a free 3′-OH moiety;
b) contacting the tissue sample with a first plurality of reversible terminator nucleotides, a first plurality of polymerases, a first plurality of caged chelator-cofactor complexes, and a first plurality of unbound caged chelator,
   wherein at least one caged chelator-cofactor complex in the first plurality comprises at least one cofactor bound to a caged chelator;
   wherein at least one reversible terminator nucleotide in the first plurality comprises the nucleotide operably linked to a cleavable 3′ terminator moiety;
c) illuminating at least one location of the tissue sample with light sufficient to uncage the caged chelator-cofactor complexes, thereby releasing the cofactors at the at least one location of the tissue sample, thereby activating the polymerases at the at least one location of the tissue sample
thereby ligating a reversible terminator nucleotide to the free 3′-OH moiety of at least one probe bound to a target analyte at the at least one location of the tissue sample, thereby extending the at least one bound probe;
d) washing the tissue sample;
e) treating the tissue sample under conditions sufficient to cleave the 3′ terminator moiety of the at least one reversible terminator nucleotide ligated in step (c), thereby exposing a free 3′-OH moiety on the extended at least one bound probe;
f) repeating steps (b)-(e) until
   at least one probe bound to a target analyte in the first location of the tissue sample has been extended such that a first spatial barcode domain has been synthesized, wherein the first spatial barcode domain comprises a unique nucleic acid sequence specific to the first location of the tissue sample,
   at least one probe bound to a target analyte in the at least second location of the tissue sample has been extended such that a second spatial barcode domain has been synthesized, wherein the second spatial barcode domain comprises a unique nucleic acid sequence specific to the at least second location of the tissue sample;
g) collecting the probes bound to target analytes in the tissue sample; and
h) quantifying via sequencing the probes collected in step (f), thereby determining the abundance of the at least two target analytes in the first and the at least second location of the tissue sample, thereby producing a spatially-resolved profile of the abundance of the at least two target analytes.

Embodiment 5. The method of embodiment 5 or embodiment 6, further comprising comparing the abundance of the at least two target analytes in the first location of the tissue sample and the at least two target analytes in the at least second location of the tissue sample.

Embodiment 6. The method of any one of the preceding embodiments, wherein the polymerase is terminal deoxynucleotidyl transferase or a biologically active fragment thereof.

Embodiment 7. The method of any one of the preceding embodiments, wherein the cleavable 3′ terminator moiety is a 3′-ONH₂ group.

Embodiment 8. The method of any one of the preceding embodiments, wherein the caged-chelator is a caged divalent cation chelator.

Embodiment 9. The method of any one of the preceding embodiments, wherein the caged-chelator is 1-(4,5-Dimethoxy-2-Nitrophenyl)-1,2-Diaminoethane-N,N,N′,N′-Tetraacetic Acid (DMNP-EDTA).

Embodiment 10. The method of any one of the preceding embodiments, wherein the cofactor is a divalent metal cofactor.

Embodiment 11. The method of any one of the preceding embodiments, wherein the cofactor is Co²⁺, Mg²⁺, Mn²⁺ Ca²⁺, Cd²⁺, Zn²⁺ or Fe²⁺.

Embodiment 12. The method of any one of the preceding embodiments, wherein the cofactor is Co²⁺.

Embodiment 13. The method of any one of the preceding embodiments, wherein contacting a tissue sample with a cofactor comprises contacting the tissue sample with a salt form of the cofactor.

Embodiment 14. The method of any one of the preceding embodiments, wherein the light sufficient to uncage the caged chelator-cofactor complex is UV light.

Embodiment 15. The method of any one of the preceding embodiments, wherein treating the tissue sample under conditions sufficient to cleave the 3′ terminator moiety of the at least one reversible terminator nucleotide comprises treating the tissue sample under acidic conditions.

Embodiment 16. The method of any one of the preceding embodiments, wherein treating the tissue sample under acidic conditions comprises contacting the tissue sample with a solution with a pH of about 5.5.

Embodiment 17. The method of any one of the preceding embodiments, wherein the probes further comprise a unique molecular identifier.

Embodiment 18. The method of any one of the preceding embodiments, wherein the probes further comprise an amplification primer binding site.

Embodiment 19. The method of any one of the preceding embodiments, wherein the amplification primer binding site is at least about 24 nucleotides in length.

Embodiment 20. The method of any one of the preceding embodiments, wherein the probes further comprise a constant region.

Embodiment 21. The method of any one of the preceding embodiments, wherein the constant region is at least about 12 to at least about 20 nucleotides in length.

Embodiment 22. The method of any one of the preceding embodiments, wherein the probes comprise, from 5′ to 3′, the target binding domain, followed by the amplification primer binding site, followed by the unique molecular identifier, followed by the target identification domain, followed by the constant region.

Embodiment 23. The method of any one of the preceding embodiments, wherein the spatial barcode domain of at least one probe bound to a target analyte in the first location of the tissue sample comprises a unique spatial identifier sequence specific to the first location of the tissue sample.

Embodiment 24. The method of any one of the preceding embodiments, wherein the spatial barcode domain of at least one probe bound to a target analyte in the at least second location of the tissue sample comprises a unique spatial identifier sequence specific to the at least second location of the tissue sample.

Embodiment 25. The method of any one of the preceding embodiments, wherein the spatial identifier sequence comprises at least about 20 nucleotides.

Embodiment 26. The method of any one of the preceding embodiments, wherein the spatial identifier sequence comprises at least about 28 nucleotides.

Embodiment 27. The method of any one of the preceding embodiments, wherein the spatial identifier sequence comprises at least four spatial identification domains.

Embodiment 28. The method of any one of the preceding embodiments, wherein each of the at least four spatial identification domains comprise the same number of nucleotides.

Embodiment 29. The method of any one of the preceding embodiments, wherein at least one of the at least four spatial identifications domains comprise a different number of nucleotides as compared to another spatial identification domain within the same spatial barcode.

Embodiment 30. The method of any one of the preceding embodiments, wherein each spatial identification domain comprises about 1 to about 4 nucleotides.

Embodiment 31. The method of any one of the preceding embodiments, wherein each spatial identification domain comprises about 4 nucleotides.

Embodiment 32. The method of any one of the preceding embodiments, wherein each of the at least four spatial identification domains comprise the same nucleotide at the 3′ terminus

Embodiment 33. The method of any one of the preceding embodiments, wherein the method further comprises, after step (f) and prior to step (g), repeating steps (b)-(e) to extend the spatial barcode domain in each location of the tissue sample such that the spatial barcode domain comprises, at the 3′ end, a delimiting domain.

Embodiment 34. The method of any one of the preceding embodiments, wherein the method further comprises, after step (f) and prior to step (g), extending the spatial barcode domain in each location of the tissue sample such that the spatial barcode domain comprises a polyT domain.

Embodiment 35. The method of any one of the preceding embodiments, wherein the method further comprises, after step (f) and prior to step (g):
(i) repeating steps (b)-(e) to extend the spatial barcode domain in each location of the tissue sample such that the spatial barcode domain comprises, a delimiting domain; and
(ii) extending the spatial barcode domain in each location of the tissue sample such that the spatial barcode domain comprises a polyT domain.

Embodiment 36. The method of any one of the preceding embodiments, wherein the delimiting domain is at least about 4 to at least about 6 nucleotides in length.

Embodiment 37. The method of any one of the preceding embodiments, wherein the sequence of the delimiting domain is the same for every spatial barcode in the sample.

Embodiment 38. The method of any one of the preceding embodiments, wherein the polyT domain comprises at least about 14 nucleotides.

Embodiment 39. The method of any one of the preceding embodiments, wherein the illumination in step (c) is provided by a light source selected from the group consisting of an arc-lamp, a laser, a focused UV light source, and light emitting diode.

Embodiment 40. The method of any one of the preceding embodiments, wherein the first location of the tissue sample and the at least second location of the tissue sample are subcellular.

Embodiment 41. The method of any one of the preceding embodiments, wherein the first location of the tissue sample and the at least second location of the tissue sample each comprise no more than one cell.

Embodiment 42. The method of any one of the preceding embodiments, wherein the first location of the tissue sample and the at least second location of the tissue sample each comprise no more than ten cells.

Embodiment 43. The method of any one of the preceding embodiments, wherein each cell within the first location of the tissue sample and the at least second location of the tissue sample are individually automatically identified and encoded.

Embodiment 44. The method of any one of the preceding embodiments, the method further comprising prior to step (a), subjecting the tissue sample to ddTTP (dideoxthymidine-triphosphate) termination.

Embodiment 45. The method of any one of the preceding embodiments, wherein subjecting the tissue sample to ddTTP termination comprises contacting the tissue sample with ddTTP and TdT.

Embodiment 46. The method of any one of the preceding embodiments, the method further comprising after step (g) and prior to step (h), amplifying the collected probes.

Embodiment 47. The method of any one of the preceding embodiments, wherein amplifying the collected probes comprises the use of a first amplification primer and a second amplification primer, wherein the first amplification primer comprises a first flow cell adapter sequence, a first NGS index sequence and a first sequencing primer binding site, and the second amplification primer comprises a second flow cell adapter sequence, a second NGS index sequence and second sequencing primer binding site.

Embodiment 48. The method of any one of the preceding embodiments, wherein at least one of the first and the second amplification primers comprises a nucleic acid sequence that is complementary to the delimiting sequence and/or the poly T domain.

### EXAMPLES

### Example 1—In Situ TdT-Mediated Nucleic Acid Extension Performed in a Spatially-Controlled Manner Using the Methods of the Present Disclosure

A flow cell was constructed with a glass slide loaded with FFPE cell pellet array section using a 75 μm thick adhesive layer and cover glass. To perform in situ TdT-mediated nucleic acid extension, the FFPE cell pellet array was contacted with a writing reaction mix comprising 2 U/uL of wildtype TdT enzyme, 1 mM CoCl₂, 1.3 mM DMNP-EDTA, 1 mM dTTP, 0.05 mM FITC-dUTP in base buffer and exposed to patterned UV illumination with 385 nm LED light source under different driving power. The base buffer for writing reaction mix was either 200 mM potassium cacodylate, 25 mM Tris, 0.1% Tween20, pH 7.9 or 50 mM potassium acetate, 20 mM Tris-acetate, pH 7.9.

UV illumination specific ROIs was done in pulses with different durations, intervals and total UV times to test different illumination conditions. A 385 nm LED was used to provide the illumination.

After UV exposure, the flow cell was washed with a wash buffer comprising 8.75×SSPE, 0.1% Tween20. The flow cell was then imaged to analyze any FITC-dUTP incorporated during the writing reaction. Furthermore, the FFPE cell pellet array was contacted with a solution comprising 20 nm of 14mer poly-A DNA molecules conjugated with Alexa546 dye for 15 min to allow hybridization of the labeled poly-A molecules to any poly-T tails incorporated during the writing reaction. After this incubation, the flow cell was washed with wash buffer. The flow cell was then imaged to analyze the any labeled poly-A molecules hybridized to the incorporated poly(T) tail in cell pellet array sample. The detection of fluorescence signal from either the incorporated FITC-dUTP or the bound Alexa546-conjugated poly(A) detection probes indicates the successful TdT-mediated tailing reaction.

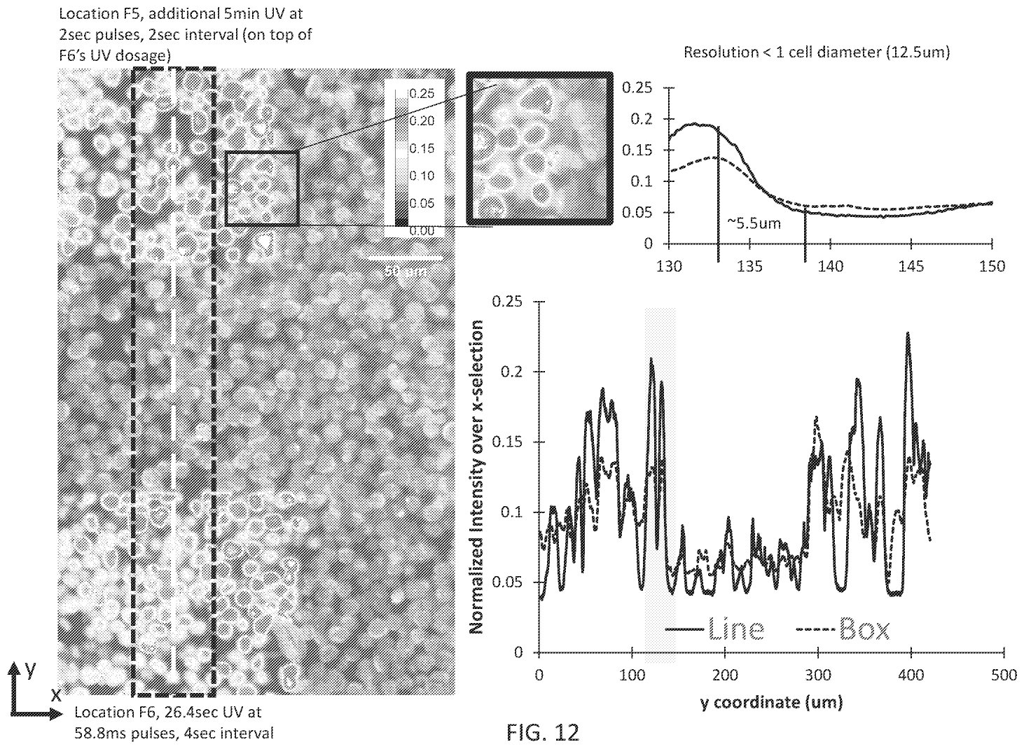

In a first experiment, in situ TdT-mediated nucleic acid extension according to the methods of the present disclosure was performed in the potassium cacodylate buffer described above at 37° C. UV illumination was provided at a power of about 4.3 W/cm² with 58.8 ms UV pulses on a 15 second interval for a total 4.294 seconds of UV exposure during a total time period of 30 minutes. Some locations on the cell pellet array received additional UV pulses of either 2 second, 10 second or 20 second duration with a 50% duty cycle for a total of 5 minute or 10 minute total additional UV exposure. No enhanced signal from locations with additional UV exposure was observed and the 26.4 seconds of UV exposure with approximately a 30-minute post-UV incubation was sufficient to perform an in situ TdT extension of 14 or more nucleotides. Following the in situ TdT extension, the cell pellet array was analyzed for FITC-dUTP incorporation, as described above. The results of this analysis are shown in FIG. 12. The results shown in FIG. 12 demonstrate that the spatial resolution of in situ TdT extension performed according to the methods of the present disclosure can be on the order of about 5.5 μm, which is less than the average diameter of one cell (about 12.5 μm). Nonlinear effects associated with Salt release by UV, TdT activation and subsequent depletion of salts create conditions that would allow the in situ synthesis of spatial barcode domains within a specific area of a tissue sample that is smaller than a single cell.

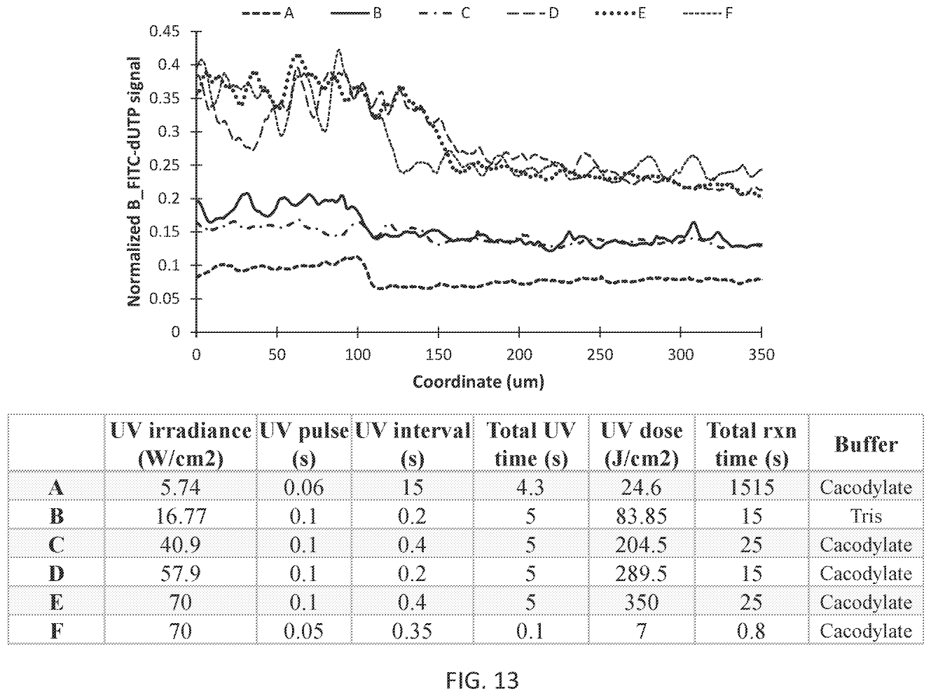

A similar experiment was performed using UV exposures of differing power, pulses, intervals and total durations, as well as different buffers (either the Tris-based or Cacodylate-based buffer described above) Again, the cell pellet array was analyzed for FITC-dUTP incorporation, as described above. The results of this analysis are shown in FIG. 13. FIG. 13 shows the normalized FITC-dUTP signal as a function of the x coordinate (top graph) for different experimental conditions (bottom table). As shown in FIG. 13, sharper writing boundaries were observed in conditions with lower UV irradiance and shorter pulses.

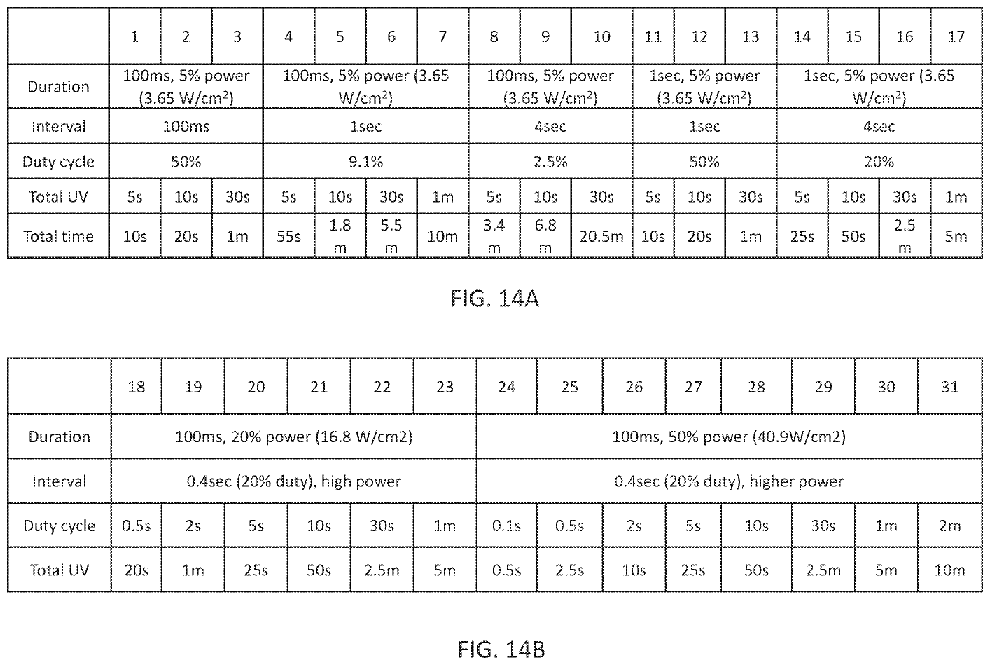

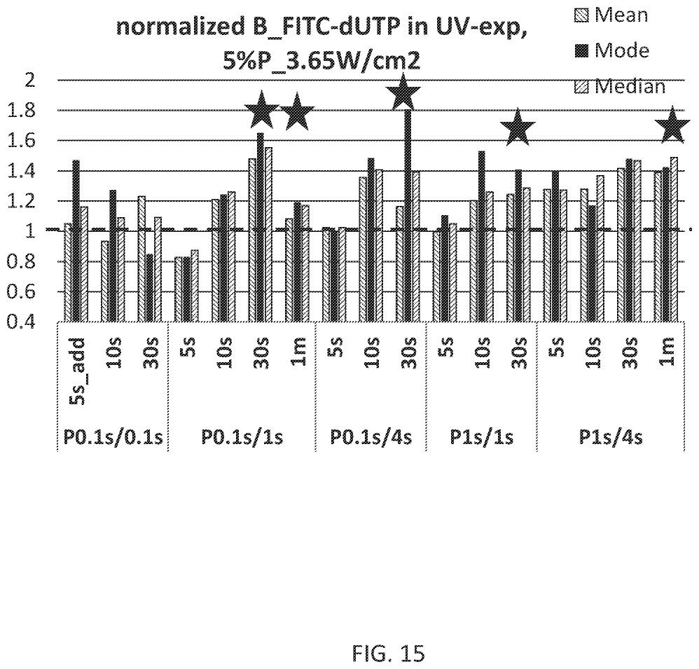

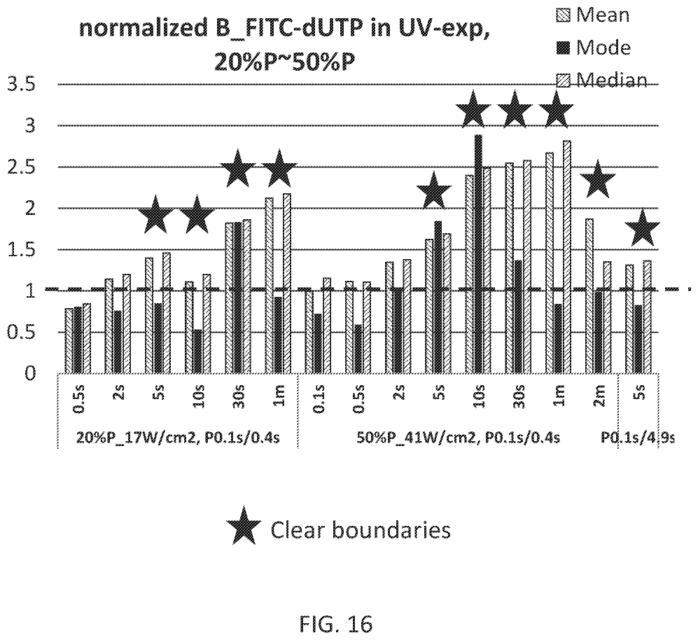

Another experiment was performed using the UV exposures of differing exposures of differing power, pulses, intervals and total durations, as put forth in FIG. 14A and FIG. 14B. The experiments were performed using the cacodylate-based buffer described above. Following the in situ TdT extension, the cell pellet array was analyzed for FITC-dUTP incorporation, as described above. The results of this analysis are shown in FIG. 15 and FIG. 16. The star symbols in FIG. 15 and FIG. 16 indicate experimental conditions where clear writing boundaries were observed. As shown in FIG. 15, when a UV power of 3.65 W/cm² was used, stronger in situ TdT mediated extension was observed when higher total UV exposure times (≥10 seconds) were used. Moreover, clear writing boundaries were observed when the total UV exposure time was ≥30 seconds. Finally, a 0.1 second interval resulted in decreased extension as compared to a 1 second or 4 second interval. Without wishing to be bound by theory, these results indicate that dNTP incorporation is the rate-limiting step. As shown in FIG. 16, when a UV power of 16.8 W/cm² or 40.9 W/cm² were used, clear writing boundaries were observed when the total UV time was ≥10 seconds, with a 0.4 second interval sufficient for longer nucleotide extensions. Moreover, the results shown in FIG. 16 indicate that higher UV power shortens the total UV exposure time that is required for in situ TdT mediated extension. Taken together, the results in FIG. 15 and FIG. 16 demonstrate that short pulses of high UV irradiance allow for the creation of clear writing boundaries in a short period of time, and that long UV-off intervals of ≥0.4 seconds results in stronger in situ TdT-mediated extension.

Taken together, the results described in this example demonstrate that the methods of the present disclosure can effectively be used to extend nucleic acids in situ in a spatially-controlled manner, with a spatial resolution that is less than the diameter of an average cell.

What is claimed is:

1. A method for in situ synthesis of a nucleic acid sequence in a tissue sample, the method comprising:
a) contacting the tissue sample with at least one probe,
wherein the probe comprises a target-binding domain and a target identification domain,
wherein the probe comprises a free 3′-OH moiety, and
wherein the target-binding domain binds to at least one target molecule located at a first location of the tissue sample;
b) contacting the tissue sample with at least one reversible terminator nucleotide, at least one polymerase, at least one caged chelator-cofactor complex, and at least one unbound caged chelator,
wherein the at least one caged chelator-cofactor complex comprises at least one cofactor bound to a caged chelator;
wherein the at least one reversible terminator nucleotide comprises the nucleotide operably linked to a cleavable 3′ terminator moiety;
c) illuminating the first location of the tissue sample with light sufficient to uncage the at least one caged chelator-cofactor complex, thereby releasing the at least one cofactor,
thereby activating the at least one polymerase,
thereby ligating the at least one reversible terminator nucleotide to the free 3′-OH moiety of the at least one bound probe;
d) washing the tissue sample;
e) treating the tissue sample under conditions sufficient to cleave the 3′ terminator moiety of the at least one reversible terminator nucleotide ligated in step (c), thereby exposing a free 3′-OH moiety on the extended at least one bound probe; and
f) repeating steps (b)-(e) until the nucleic acid sequence has been synthesized.

2. The method of claim 1, wherein the target-binding domain binds to at least one target molecule at an at least second location of the tissue sample, and wherein the method further comprises repeating steps (b)-(f) at the at least second location.

3. The method of any one of the preceding claims, wherein the nucleic acid sequence synthesized at the first location of the tissue sample is different than the nucleic acid sequence synthesized at the at least second location of the tissue sample.

4. A method of producing a spatially-resolved profile of the abundance of at least two target analytes in a first and an at least second location of a tissue sample comprising:
a) contacting the tissue sample with a solution comprising at least two species of probes, the probes comprising a target-binding domain and a target identification domain,
wherein each species of probe comprises a unique target-binding domain that binds to one of the at least two target analytes and a unique target identification domain specific for the target analyte, and a free 3′-OH moiety;
b) contacting the tissue sample with a first plurality of reversible terminator nucleotides, a first plurality of polymerases, a first plurality of caged chelator-cofactor complexes, and a first plurality of unbound caged chelator,
wherein at least one caged chelator-cofactor complex in the first plurality comprises at least one cofactor bound to a caged chelator;
wherein at least one reversible terminator nucleotide in the first plurality comprises the nucleotide operably linked to a cleavable 3′ terminator moiety;
c) illuminating at least one location of the tissue sample with light sufficient to uncage the caged chelator-cofactor complexes, thereby releasing the cofactors at the at least one location of the tissue sample,
thereby activating the polymerases at the at least one location of the tissue sample
thereby ligating a reversible terminator nucleotide to the free 3′-OH moiety of at least one probe bound to a target analyte at the at least one location of the tissue sample, thereby extending the at least one bound probe;
d) washing the tissue sample;
e) treating the tissue sample under conditions sufficient to cleave the 3′ terminator moiety of the at least one reversible terminator nucleotide ligated in step (c), thereby exposing a free 3′-OH moiety on the extended at least one bound probe;
f) repeating steps (b)-(e) until
at least one probe bound to a target analyte in the first location of the tissue sample has been extended such that a first spatial barcode domain has been synthesized, wherein the first spatial barcode domain comprises a unique nucleic acid sequence specific to the first location of the tissue sample,
at least one probe bound to a target analyte in the at least second location of the tissue sample has been extended such that a second spatial barcode domain has been synthesized, wherein the second spatial barcode domain comprises a unique nucleic acid sequence specific to the at least second location of the tissue sample;
g) collecting the probes bound to target analytes in the tissue sample; and
h) quantifying via sequencing the probes collected in step (f), thereby determining the abundance of the at least two target analytes in the first and the at least second location of the tissue sample, thereby producing a spatially-resolved profile of the abundance of the at least two target analytes.

5. The method of claim 5 or claim 6, further comprising comparing the abundance of the at least two target analytes in the first location of the tissue sample and the at least two target analytes in the at least second location of the tissue sample.

6. The method of any one of the preceding claims, wherein the polymerase is terminal deoxynucleotidyl transferase or a biologically active fragment thereof.

7. The method of any one of the preceding claims, wherein the cleavable 3′ terminator moiety is a 3′-ONH₂ group.

8. The method of any one of the preceding claims, wherein the caged-chelator is a caged divalent cation chelator, preferably wherein the caged-chelator is 1-(4,5-Dimethoxy-2-Nitrophenyl)-1,2-Diaminoethane-N,N,N′,N′-Tetraacetic Acid (DMNP-EDTA).

9. The method of any one of the preceding claims, wherein the cofactor is a divalent metal cofactor, preferably wherein the cofactor is Co²⁺, Mg²⁺, Mn²⁺ Ca²⁺, Cd²⁺, Zn²⁺ or Fe²⁺, preferably wherein the cofactor is Co²⁺.

10. The method of any one of the preceding claims, wherein the light sufficient to uncage the caged chelator-cofactor complex is UV light.

11. The method of any one of the preceding claims, wherein treating the tissue sample under conditions sufficient to cleave the 3′ terminator moiety of the at least one reversible terminator nucleotide comprises treating the tissue sample under acidic conditions, preferably wherein treating the tissue sample under acidic conditions comprises contacting the tissue sample with a solution with a pH of about 5.5.

12. The method of any one of the preceding claims, wherein the probes further comprise:
i) a unique molecular identifier;
ii) an amplification primer binding site;
iii) a constant region; or
iv) any combination of the preceding,
preferably wherein the probes comprise, from 5′ to 3′, the target binding domain, followed by the amplification primer binding site, followed by the unique molecular identifier, followed by the target identification domain, followed by the constant region.

13. The method of any one of the preceding claims, wherein the spatial barcode domain of at least one probe bound to a target analyte in the first location of the tissue sample comprises a unique spatial identifier sequence specific to the first location of the tissue sample, and/or
the spatial barcode domain of at least one probe bound to a target analyte in the at least second location of the tissue sample comprises a unique spatial identifier sequence specific to the at least second location of the tissue sample.

14. The method of any one of the preceding claims, wherein the spatial identifier sequence comprises
i) at least about 20 nucleotides; or
ii) at least about 28 nucleotides.

15. The method of any one of the preceding claims, wherein the spatial identifier sequence comprises at least four spatial identification domains, preferably wherein
i) each of the at least four spatial identification domains comprise the same number of nucleotides; or
ii) at least one of the at least four spatial identifications domains comprise a different number of nucleotides as compared to another spatial identification domain within the same spatial barcode.

16. The method of any one of the preceding claims, wherein each spatial identification domain comprises about 1 to about 4 nucleotides, preferably wherein each spatial identification domain comprises about 4 nucleotides.

17. The method of any one of the preceding claims, wherein each of the at least four spatial identification domains comprise the same nucleotide at the 3′ terminus

18. The method of any one of the preceding claims, wherein the method further comprises, after step (f) and prior to step (g), repeating steps (b)-(e) to extend the spatial barcode domain in each location of the tissue sample such that the spatial barcode domain comprises, at the 3′ end, a delimiting domain.

19. The method of any one of the preceding claims, wherein the method further comprises, after step (f) and prior to step (g), extending the spatial barcode domain in each location of the tissue sample such that the spatial barcode domain comprises a polyT domain.

20. The method of any one of the preceding claims, wherein the method further comprises, after step (f) and prior to step (g):
(i) repeating steps (b)-(e) to extend the spatial barcode domain in each location of the tissue sample such that the spatial barcode domain comprises, a delimiting domain; and
(ii) extending the spatial barcode domain in each location of the tissue sample such that the spatial barcode domain comprises a polyT domain.

21. The method of any one of the preceding claims, wherein the sequence of the delimiting domain is the same for every spatial barcode in the sample.

22. The method of any one of the preceding claims, wherein the first location of the tissue sample and the at least second location of the tissue sample are subcellular.

23. The method of any one of the preceding claims, wherein the first location of the tissue sample and the at least second location of the tissue sample each
i) comprise no more than one cell; or
ii) comprise no more than ten cells.

24. The method of any one of the preceding claims, the method further comprising prior to step (a), subjecting the tissue sample to ddTTP (dideoxthymidine-triphosphate) termination, preferably wherein subjecting the tissue sample to ddTTP termination comprises contacting the tissue sample with ddTTP and TdT.

25. The method of any one of the preceding claims, the method further comprising after step (g) and prior to step (h), amplifying the collected probes, preferably wherein amplifying the collected probes comprises the use of a first amplification primer and a second amplification primer, wherein
the first amplification primer comprises a first flow cell adapter sequence, a first NGS index sequence and a first sequencing primer binding site, and
the second amplification primer comprises a second flow cell adapter sequence, a second NGS index sequence and second sequencing primer binding site.

26. The method of any one of the preceding claims, wherein at least one of the first and the second amplification primers comprises a nucleic acid sequence that is complementary to the delimiting sequence and/or the polyT domain.
